(19) United States
(12) Patent Application Publication
(10) Pub. No.: US 2023/0027075 A1
(43) Pub. Date: Jan. 26, 2023

(54) SULFONAMIDE COMPOUNDS TARGETING CD73 AND ADENOSINE RECEPTORS

(71) Applicant: Aurigene Discovery Technologies Limited, Bangalore (IN)

(72) Inventors: Susanta SAMAJDAR, Bangalore (IN); Dinesh CHIKKANNA, Bangalore (IN); Sunil Kumar PANIGRAHI, Boudh (IN); Chandregowda VENKATESHAPPA, Bangalore (IN); Garima PRIYADARSHANI, Patna (IN)

(21) Appl. No.: 17/779,973

(22) PCT Filed: Nov. 26, 2020
(86) PCT No.: PCT/IB2020/061172

§ 371 Date: May 25, 2022

(30) Foreign Application Priority Data
Nov. 26, 2019 (IN) 201941048228

(51) Int. Cl. C07D 487/04

(52) U.S. Cl. CPC C07D 487/04

(57) ABSTRACT

The present invention relates to bispecific compound of formula (I) as dual inhibitors of CD73 and adenosine receptors. The present invention also relates to pharmaceutical compositions comprising said compounds or a pharmaceutically acceptable salt or a stereoisomer or a prodrug thereof and use of such compounds in the treatment of diseases mediated by CD73 and/or adenosine receptors, particularly A2aR or A2bR.

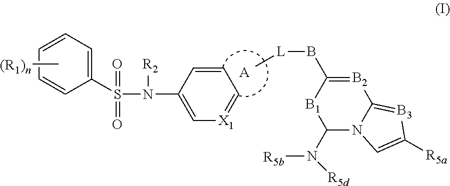

### RELATED APPLICATION

This application claims the benefit of Indian provisional application number 201941048228, filed on 26 Nov. 2019; the specification of which is hereby incorporated by reference in their entirety.

### FIELD OF THE INVENTION

The present invention relates to sulfonamide compounds represented by compound of formula (I), pharmaceutical compositions thereof and a method of preparation of the said compounds. The present invention also relates to a use of compound of formula (I) or a pharmaceutically acceptable thereof, as dual inhibitors of CD73 and adenosine receptors and for the treatment of diseases mediated by CD73 and adenosine receptors.

### BACKGROUND OF THE INVENTION

The anticancer immune response involves extracellular ATP to block cell proliferation through T-cell activation. However, in the tumor micro-environment, two extracellular membrane-bound enzymes (CD39 and CD73) are overexpressed and hydrolyze efficiently ATP into AMP then further into immune-suppressive adenosine once generated through the activity of CD39 and CD73, adenosine binds to A2A and A2B receptors expressed on tumor cells. This signaling can enhance tumor growth and directly promotes tumor cell proliferation. Additionally, adenosine inhibits anti-tumor cell activity through the inhibition of CD4+ cells, T cells, CTLs, dendritic cells and NK cells. Adenosine also activates immunosuppressive cells such as Tregs, myeloid-derived suppressor cells (MDSCs) and tumor-associated macrophages (TAMs), allowing additional suppression of anti-tumor activity.

CD73 (designated also as ecto-5′ nucleotidase or ecto5′NTase) appears to be a clinically key target in the management of cancer. Targeting A2A or A2B receptors or inhibiting adenosine signaling, via CD73 blockade, can represent a promising adjunct to tumor immunotherapy since several immunotherapeutic approaches to curb neoplasia have failed due to CD73 over expression in cancer cells or high adenosine levels within tumor microenvironment. The genetic deletion of immunosuppressive A2A and A2B adenosine receptors or their pharmacological inactivation can prevent the inhibition of antitumor T cells by the hypoxic tumor microenvironment, thus facilitating a process of full cancer rejection.

This indicates a crucial role of the CD73/adenosine/A2A-A2B receptor axis in protecting normal and cancerous tissues from collateral damage during immune responses. In particular, evidence for the role of extracellular adenosine and thus for the relevance of CD73 and A2A receptor in tumor protection.

Recent genetic and pharmacological studies using two separate inhibitors have established that co-blockade of CD73 and A2aR leads to more potent anti-tumor activity than blockade of either, partly due to increased CD73 expression in the absence of A2aR (Young et al., 2016, Cancer Cell 30, 391-403, Sep. 12, 2016).

The key factor influenced in the design of CD73 inhibitor: a) with antibodies targeting CD73, as with other antibody targets, the capacity for engaging Fc receptors can be reduced by antibody engineering. In that case, the widespread expression of CD73 in normal tissues could lead to safety concerns using an antibody capable of Fc receptor-mediated antibody-dependent cellular cytotoxicity. For example, considering the above reason, MEDI9447 was introduced with mutations abrogating Fc engagement, but in contrast, this reduced the efficacy of MEDI9447 which shows the clear positive role in antitumor immunity demonstrating for Fc engagement with anti-CD73 antibodies; b) on contrast, small molecules display several advantages as compared with mAb approaches, such as oral bioavailability, a greater exposure of the tumor microenvironment and the chance of different formulations to overcome pharmacokinetic and/or pharmacodynamic challenges.

For small molecules, it is important to consider whether the molecule is competitive or noncompetitive with AMP in the tumor microenvironment as noncompetitive inhibitors are expected to be active independently of extracellular AMP but, by contrast, the efficacy of competitive inhibitors could be reduced by the presence of a high concentration of the endogenous substrate. Though the concept of bispecific monoclonal antibody which is an engineered molecule that binds to two different types of antigen at same time is evolving, small molecule directed against two targets is exceptional.

The objective of present invention is dual target approach by designing a bispecific small molecule with integrated pharmacophore being able to bind to “CD73” and “adenosine receptor” and to achieve a comparable efficacy with a single molecule than existing monospecific compound by targeting CD73 through the noncompetitive/allosteric inhibitors approach and A2aR as orthosteric binding site which might be an optimal way to completely blunt adenosine's pro-tumorigenic actions.

### SUMMARY OF THE INVENTION

Provided herein bispecific compounds and pharmaceutical compositions thereof used for the treatment of diseases or disorders mediated by CD73 and adenosine receptor.

In one aspect, the present invention provides compound of formula (I):

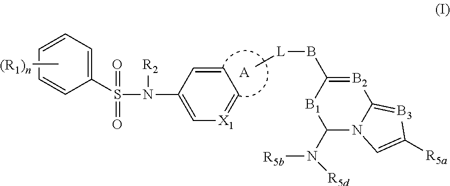

or a pharmaceutically acceptable salt or a stereoisomer or a prodrug thereof;

wherein,
X₁ is C or N;
A is optionally fused 5- to 6-membered heteroaryl ring containing 1 or 2 heteroatoms selected from N, O and S; wherein if A is absent, L is attached to 6-membered ring containing X₁;
L represents alkylene, alkenylene or alkynylene, wherein one or more C atoms are replaced with N or O; and each of alkylene, alkenylene and alkynylene is optionally substituted with one, two or three substituents selected from halo, hydroxyl, haloalkyl, amino, amido, alkyl, aryl, cycloalkyl, heteroaryl and heterocycloalkyl;
B represents —O— or —NR_{5d}—;
each B₁, B₂ and B₃ independently represents —N— or —CX₂—;
X₂ represents hydrogen, alkyl, cycloalkyl, aryl, 5- or 6-membered heterocycloalkyl or 5- or 6-membered heteroaryl;
R₁ at each occurrence independently represents alkyl, —NRₐR_{b}, halo, haloalkyl, —CONRₐR_{b}, —ORₐ, cycloalkyl, aryl, heteroaryl or heterocycloalkyl; wherein each of cycloalkyl, aryl, heteroaryl and heterocycloalkyl is substituted with one, two or three occurrences of R₃;
alternatively, any two R₁ groups, bonded to adjacent carbon atoms, combine together to form a 5- or 6-membered heterocycloalkyl ring containing 1 or 2 heteroatoms selected from N, O and S; wherein the said heterocycloalkyl is substituted with one, two or three occurrences of R₃;
R₂ represents hydrogen, halo, alkyl, hydroxyl or cycloalkyl;
R₃ represents hydrogen, oxo, halo, amino, alkyl, amido, hydroxyl, cycloalkyl, aryl, heteroaryl or heterocycloalkyl;
R₄ represents hydrogen, hydroxyl, halo or alkyl;
Rₐ and R_{b}, each independently represents hydrogen, alkyl, haloalkyl, ester, —COO— alkyl, Aaa or —CO-Aaa; wherein 1 or 2 C atoms in the said alkyl chain are optionally replaced with O; and the said alkyl is optionally substituted with alkoxy or oxo;
Aaa is an amino acid residue selected from Ala, Ser, Thr, Cys, Val, Leu and Ile; wherein the C-terminus thereof is a free terminus, is amidated or is esterified; and the N-terminus thereof is a free terminus or Boc-protected;
R₅ₐ is aryl or 5- or 6-membered heteroaryl;
R_{5b}, R_{5c} and R_{5d} each independently represents hydrogen, alkyl, acyl, ester, —COO— alkyl, cycloalkyl, aryl, aralkyl, heterocycloalkyl, heteroaryl or heteroaralkyl; wherein 1 or 2 C atoms in the said alkyl chain are optionally replaced with O; and the said alkyl is optionally substituted with alkoxy or oxo;
‘n’ is an integer selected from 0, 1, 2, 3 and 4.

In yet another aspect, the present invention provides a pharmaceutical composition comprising a compound of formula (I) or a pharmaceutically acceptable salt or a stereoisomer or a prodrug thereof and at least one pharmaceutically acceptable excipient (such as a pharmaceutically acceptable carrier or diluent).

In another aspect, the present invention provides a pharmaceutical composition for the treatment of diseases or conditions that are dependent upon inhibiting the activity of CD73 and blocking the signaling of adenosine receptor.

In yet another aspect, the present invention relates to the preparation of compound of formula (I) or a pharmaceutically acceptable salt or a stereoisomer or a prodrug thereof.

In another aspect, the present invention provides methods of treating a cancer by administering a therapeutically effective amount of a compound of formula (I) or a pharmaceutically acceptable salt or a stereoisomer or a prodrug thereof to a subject, e.g., a human, in need thereof.

### DETAILED DESCRIPTION OF THE INVENTION

The present invention relates to bispecific compound acting as a dual inhibitor of CD73 and adenosine receptors and pharmaceutical compositions comprising said compounds.

The present invention also relates to a use of said compounds and composition comprising said compounds for the treatment and/or prevention of diverse array of diseases and disorders mediated by CD73 and adenosine receptor.

In certain embodiments, the present invention provides compound of formula (I),

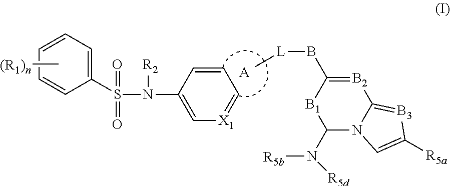

or a pharmaceutically acceptable salt or a stereoisomer or a prodrug thereof;

wherein,
X₁ is C or N;
A is optionally fused 5- to 6-membered heteroaryl ring containing 1 or 2 heteroatoms selected from N, O and S; wherein if A is absent, L is attached to 6-membered ring containing X₁;
L represents alkylene, alkenylene or alkynylene, wherein one or more C atoms are replaced with N or O; and each of alkylene, alkenylene and alkynylene is optionally substituted with one, two or three substituents selected from halo, hydroxyl, haloalkyl, amino, amido, alkyl, aryl, cycloalkyl, heteroaryl and heterocycloalkyl;
B represents —O— or —NR_{5d}—;
each B₁, B₂ and B₃ independently represents —N— or —CX₂—;
X₂ represents hydrogen, alkyl, cycloalkyl, aryl, 5- or 6-membered heterocycloalkyl or 5- or 6-membered heteroaryl;
R₁ at each occurrence independently represents alkyl, —NRₐR_{b}, halo, haloalkyl, CONRₐR_{b}, —ORₐ, cycloalkyl, aryl, heteroaryl or heterocycloalkyl; wherein each of cycloalkyl, aryl, heteroaryl and heterocycloalkyl is substituted with one, two or three occurrences of R₃;
alternatively, any two R₁ groups, bonded to adjacent carbon atoms, combine together to form a 5- or 6-membered heterocycloalkyl ring containing 1 or 2 heteroatoms selected from N, O and S; wherein the said heterocycloalkyl is substituted with one, two or three occurrences of R₃;
R₂ represents hydrogen, halo, alkyl, hydroxyl or cycloalkyl;
R₃ represents hydrogen, oxo, halo, amino, alkyl, amido, hydroxyl, cycloalkyl, aryl, heteroaryl or heterocycloalkyl;
R₄ represents hydrogen, hydroxyl, halo or alkyl;
Rₐ and R_{b}, each independently represents hydrogen, alkyl, haloalkyl, ester, —COO— alkyl, Aaa or —CO-Aaa; wherein 1 or 2 C atoms in the said alkyl chain are optionally replaced with O; and the said alkyl is optionally substituted with alkoxy or oxo;
Aaa is an amino acid residue selected from Ala, Ser, Thr, Cys, Val, Leu and Ile; wherein the C-terminus thereof is a free terminus, is amidated or is esterified; and the N-terminus thereof is a free terminus or Boc-protected;
R₅ₐ is aryl or 5- or 6-membered heteroaryl;
R_{5b}, R_{5c} and R_{5d} each independently represents hydrogen, alkyl, acyl, ester, —COO— alkyl, cycloalkyl, aryl, aralkyl, heterocycloalkyl, heteroaryl or heteroaralkyl; wherein 1 or 2 C atoms in the said alkyl chain are optionally replaced with O; and the said alkyl is optionally substituted with alkoxy or oxo;
‘n’ is an integer selected from 0, 1, 2, 3 and 4.

In certain embodiments, R₁, at each occurrence, independently represents alkyl, halo, haloalkyl, —CONH₂, —OH or —OCO-Aaa. In certain embodiments, R₁, at each occurrence, independently represents —CH₃, —F, —Cl, —CF₃, —CONH₂, —OH or OCONHCH[CH(CH₃)₂]COOCH₃.

In certain embodiments, any two R₁ groups, bonded to adjacent carbon atoms, combine together to form a 5- or 6-membered heterocycloalkyl ring containing 1 or 2 heteroatoms selected from N, O and S; wherein the said heterocycloalkyl is substituted with one, two or three occurrences of R₃; wherein R₃ represents oxo, halo, alkyl or hydroxyl.

In certain embodiments, any two R₁ groups, bonded to adjacent carbon atoms, combine together to form

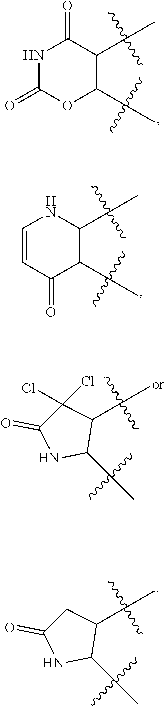 [SMILES: *C1OC(=O)NC(=O)C1* ; *C1NC=CC(=O)C1* ; *C1NC(=O)C(Cl)(Cl)C1* ; *C1CC(=O)NC1*]

In certain embodiments, R₂ is hydrogen, halo, alkyl or hydroxyl. In certain embodiments of formula (I), R₂ is hydrogen or alkyl. In certain particular embodiments of formula (I), R₂ is hydrogen.

In certain embodiments, R₄ represents hydrogen, hydroxyl or halo. In certain embodiments, R₄ represents hydrogen, hydroxyl, —F or —Cl.

In certain embodiments, A is fused 5-membered heteroaryl ring selected from furan, thiophene, pyrrole, pyrazole, imidazole, oxazole, isoxazole, thiazole and isothiazole. In certain embodiments, A is fused pyrrole ring or fused pyrazole ring. In certain embodiments, if A is absent, L is attached to 6-membered ring containing X₁. In certain embodiments, if A is fused 5-membered heteroaryl ring, R₄ is substituted in A ring.

In certain embodiments, L represents —(C₁-C₆)alkylene-, —(C₂-C₆)alkenylene- or —(C₂-C₆)alkynylene-, wherein one or more C atoms are replaced with N or O atom. In certain embodiments, L represents —(C₁-C₆)alkylene- wherein one or more —CH₂— groups are replaced with —NH— or —O— groups.

In certain embodiments, L represents —CH₂—, —(CH₂—CH₂)—, —(CH₂—CH₂—CH₂)—, —(CH₂—CH₂—CH₂—CH₂)—, —NHCH₂—, —NH(CH₂—CH₂)—, —NH(CH₂—CH₂—CH₂)—, —OCH₂—, —O—CH₂—CH₂—, —O—CH₂—CH₂—CH₂—. In certain embodiments, L represents —CH₂—, —(CH₂—CH₂)—, —(CH₂—CH₂—CH₂)—, —(CH₂—CH₂—CH₂—CH₂)— or —NH(CH₂—CH₂)—.

In certain embodiments, B represents —O—, —NH— or —N(CH₃)—.

In certain embodiments, each B₁, B₂ and B₃ independently represents —N— or —CH—.

In certain embodiments, R₅ₐ is 5- or 6-membered heteroaryl. In certain embodiments, R₅ₐ is 5-membered heteroaryl. In certain embodiments, R₅ₐ is furanyl, thienyl, pyrrolyl, pyrazolyl, imidazolyl, oxazolyl, isoxazolyl, thiazolyl, isothiazolyl, 1H-tetrazolyl, oxadiazolyl or triazolyl. In certain embodiments, R₅ₐ is furanyl.

In certain embodiments, R_{5b} and R_{5c} each independently represents hydrogen, acyl or ester. In certain embodiments, R_{5b} is hydrogen. In certain embodiments, R_{5c} represents hydrogen, acyl or ester.

In certain embodiments, the present invention provides the compound of formula (I) or a pharmaceutically acceptable salt or a stereoisomer or a prodrug thereof; wherein,
X₁ is C or N;
R₁, at each occurrence, independently represents —CH₃, —F, —Cl, —CF₃, —CONH₂, —OH or —OCONHCH[CH(CH₃)₂]COOCH₃; or any two R₁ groups, bonded to adjacent carbon atoms, combine together to form

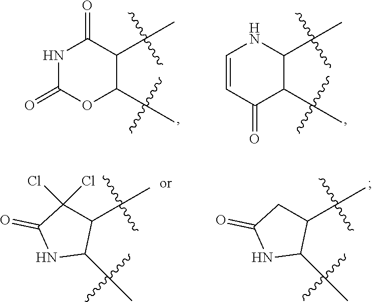 [SMILES: *C1OC(=O)NC(=O)C1* ; *C1NC=CC(=O)C1* ; *C1NC(=O)C(Cl)(Cl)C1* ; *C1CC(=O)NC1*]

R₂ is hydrogen;

R₄ represents hydrogen, hydroxyl, —F or —Cl;

A is fused pyrrole ring or fused pyrazole ring;

L represents —CH₂—, —(CH₂—CH₂)—, —(CH₂—CH₂—CH₂)—, —(CH₂—CH₂—CH₂—CH₂)— or —NH(CH₂—CH₂)—;

B represents —O—, —NH— or —N(CH₃)—;

B₁, B₂ and B₃ independently represents —N— or —CH—;

R₅ₐ is furanyl, thienyl, pyrrolyl, pyrazolyl, imidazolyl, oxazolyl, isoxazolyl, thiazolyl, isothiazolyl, 1H-tetrazolyl, oxadiazolyl or triazolyl;

R_{5b} represents hydrogen, acyl or —COOCH₂CH(CH₃)₂;

R_{5c} is hydrogen;

‘n’ is an integer selected from 0, 1, 2 and 3.

In certain embodiments, the present invention provides the compound of formula (I) or a pharmaceutically acceptable salt thereof, wherein,
X₁ is C or N;
A is optionally fused 5- to 6-membered heteroaryl ring containing 1 or 2 heteroatoms selected from N, O and S; wherein if A is absent, L is attached with 6-membered ring containing X₁;
L represents alkylene, alkenylene or alkynylene, wherein one or more C atoms are replaced with N or O; and each of alkylene, alkenylene and alkynylene is optionally substituted with one, two or three substituents selected from hydrogen, halo, hydroxyl, haloalkyl, amino, amido, alkyl, aryl, cycloalkyl, heteroaryl and heterocycloalkyl;
B represents —O— or —NR_{5d}—;
each B₁, B₂ and B₃ independently represents —N— or —CX₂—;
X₂ represents hydrogen, alkyl, cycloalkyl, aryl, 5- or 6-membered heterocycloalkyl or 5- or 6-membered heteroaryl;
R₁ at each occurrence independently represents alkyl, —NRₐR_{b}, halo, haloalkyl, —CONRₐR_{b}, —ORₐ, cycloalkyl, aryl, heteroaryl or heterocycloalkyl; wherein each of cycloalkyl, aryl, heteroaryl and heterocycloalkyl is substituted with one, two or three occurrences of R₃;
alternatively, any two R₁ groups, bonded to adjacent carbon atoms, combine together to form a 5- or 6-membered heterocycloalkyl ring containing 1 or 2 heteroatoms selected from N, O and S; wherein the said heterocycloalkyl is substituted with one, two or three occurrences of R₃;
R₂ represents hydrogen, halo, alkyl, hydroxyl or cycloalkyl;
R₃ represents hydrogen, oxo, halo, amino, alkyl, amido, hydroxyl, cycloalkyl, aryl, heteroaryl or heterocycloalkyl;
R₄ represents hydrogen, hydroxyl, halo or alkyl;
Rₐ and R_{b}, each independently represents hydrogen, alkyl or haloalkyl;
R₅ₐ is aryl or 5- or 6-membered heteroaryl;
R_{5b}, R_{5c} and R₅ₐ each independently represents hydrogen, alkyl, acyl, ester, cycloalkyl, aryl, aralkyl, heterocycloalkyl, heteroaryl or heteroaralkyl;
‘n’ is an integer selected from 0, 1, 2, 3 and 4.

In certain embodiments, the present invention provides compound of formula (IA),

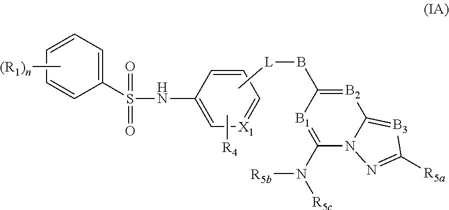

or a pharmaceutically acceptable salt or a stereoisomer or a prodrug thereof; wherein,
X₁, R₁, R₄, L, B, B₁, B₂, B₃, R₅ₐ, R_{5b}, R_{5c} and ‘n’ are as defined in compound of formula (I).

In certain embodiments of formula (IA), B represents —O— or —NR_{5d}—; and R₅ₐ represents hydrogen or alkyl. In certain embodiments of formula (IA), B represents —O—, —NH— or —NCH₃—.

In certain embodiments of formula (IA), B represents —O— or —NH.

In certain embodiments of formula (IA), R₄ is hydrogen, hydroxyl or halo. In certain embodiments of formula (IA), R₄ is hydrogen, hydroxyl, —F or —Cl.

In certain embodiments of formula (IA), R₅ₐ is phenyl or 5- or 6-membered heteroaryl. In certain embodiments of formula (IA), R₅ₐ is 5- or 6-membered heteroaryl. In certain embodiments of formula (IA), R₅ₐ is 5-membered heteroaryl. In certain embodiments of formula (IA), R₅ₐ is furanyl, thienyl, pyrrolyl, pyrazolyl, imidazolyl, oxazolyl, isoxazolyl, thiazolyl, isothiazolyl, 1H-tetrazolyl, oxadiazolyl or triazolyl. In certain embodiments of formula (IA), R₅ₐ is furanyl.

In certain embodiments of formula (IA), R_{5b} and R_{5c} each independently represents hydrogen, alkyl, acyl, ester, cycloalkyl, aryl, aralkyl, 5- or 6-membered heterocycloalkyl, 5- or 6-membered heteroaryl or heteroaralkyl. In certain embodiments of formula (IA), R_{5b} and R_{5c} each independently represents hydrogen, acyl or ester. In certain embodiments of formula (IA), R_{5c} is hydrogen. In certain embodiments, R_{5b} represents hydrogen, acyl or ester. In certain embodiments, R_{5b} represents hydrogen, acyl or —COOCH₂CH(CH₃)₂.

In certain embodiments of formula (IA), L represents —(C₁-C₆)alkylene-, wherein one or two C atoms are replaced with N or 0.

In certain embodiments of formula (IA), L represents —(CH₂)ₘ— wherein m represents an integer selected from 1, 2, 3 or 4. In certain embodiments, L represents —(CH₂)₁₋₄—. In certain embodiments, L represents —CH₂—, —(CH₂—CH₂)—, —(CH₂—CH₂—CH₂)— or —(CH₂—CH₂—CH₂—CH₂)—.

In certain embodiments of formula (IA), L represents —NH(CH₂)₁₋₃— wherein m represents an integer selected from 1, 2 or 3. In certain embodiments, L represents —NH—(CH₂)₁₋₃—. In certain embodiments, L represents —NHCH₂—, —NH(CH₂—CH₂)— or —NH(CH₂—CH₂—CH₂)—.

In certain embodiments, the present invention provides a compound of formula (IA) or a pharmaceutically acceptable salt or a stereoisomer or a prodrug thereof; wherein

X₁ is C or N;

R₁, at each occurrence, independently represents —CH₃, —F, —Cl, —CF₃, —CONH₂, —OH or —OCONHCH[CH(CH₃)₂]COOCH₃; or any two R₁ groups, bonded to adjacent carbon atoms, combine together to form

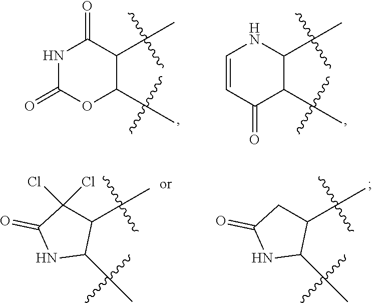 [SMILES: *C1OC(=O)NC(=O)C1* ; *C1NC=CC(=O)C1* ; *C1NC(=O)C(Cl)(Cl)C1* ; *C1CC(=O)NC1*]

R₄ represents hydrogen, hydroxyl, —F or —Cl;

L represents —CH₂—, —(CH₂—CH₂)—, —(CH₂—CH₂—CH₂)—, —(CH₂—CH₂—CH₂—CH₂)— or —NH(CH₂—CH₂)—;

B represents —O—, —NH— or —N(CH₃)—;

B₁, B₂ and B₃ independently represents —N— or —CH—;

R₅ₐ is furanyl, thienyl, pyrrolyl, pyrazolyl, imidazolyl, oxazolyl, isoxazolyl, thiazolyl, isothiazolyl, 1H-tetrazolyl, oxadiazolyl or triazolyl;

R_{5b} represents hydrogen, acyl or —COOCH₂CH(CH₃)₂;

R_{5c} is hydrogen;

‘n’ is an integer selected from 0, 1, 2 and 3.

In certain embodiments, the present invention provides compound of formula (IB),

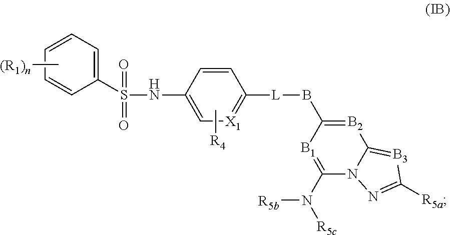

or a pharmaceutically acceptable salt or a stereoisomer or a prodrug thereof; and

X₁, R₁, R₄, L, B, B₁, B₂, B₃, R₅ₐ, R_{5b}, R_{5c} and ‘n’ are as defined in compound of formula (I).

In certain embodiments, R₁ at each occurrence independently represents alkyl, —NRₐR_{b}, halo, haloalkyl, —CONH₂ or —ORₐ; wherein Rₐ and R_{b} independently represents hydrogen, Aaa or —CO-Aaa. In certain embodiments, Aaa is an amino acid residue selected from Ala, Ser, Thr, Cys, Val, Leu and Ile; wherein the C-terminus thereof is a free terminus, is amidated or is esterified; and the N-terminus thereof is a free terminus or Boc-protected.

In certain embodiments, Rₐ is hydrogen or —CO-Aaa; wherein Aaa is Val residue in which the C-terminus is esterified.

In certain embodiments, Rₐ is hydrogen or —CO-Aaa; wherein Aaa is Val residue in which C-terminus is free (e.g. CO₂H form).

In certain embodiments, Rₐ is hydrogen or —CO-Aaa; wherein Aaa is Val residue in which C-terminus is free (e.g. —CO₂-alkyl form).

In certain embodiments, Rₐ is hydrogen or —CO-Aaa; wherein Aaa is Val residue in which C-terminus is free (e.g. CONH₂ form). In certain embodiments of formula (IB), R₁, at each occurrence, independently represents —CH₃, —F, —Cl, —CF₃, —CONH₂, —OH or —OCONHCH[CH(CH₃)₂]COOCH₃.

In certain embodiments of formula (IB), any two R₁ groups, bonded to adjacent carbon atoms, combine together to form

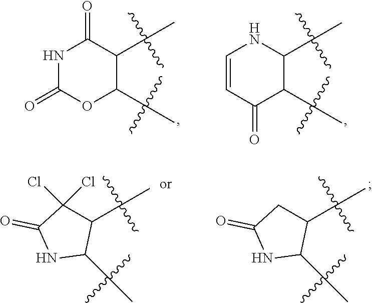 [SMILES: *C1OC(=O)NC(=O)C1* ; *C1NC=CC(=O)C1* ; *C1NC(=O)C(Cl)(Cl)C1* ; *C1CC(=O)NC1*]

In certain embodiments, L represents —(C₁-C₆)alkylene- or —(C₂-C₆)alkenylene- wherein one or more C atoms of (C₁-C₆)alkylene and (C₂-C₆)alkenylene groups are replaced with N or O atom.

In certain embodiments, B₁ and B₃ each independently represents —N— or —CX₂—; B₂ represents —N—; wherein X₂ is as defined in compound of formula (I). In certain embodiments of formula (IB), B₁, B₂ and B₃ independently represents —N— or —CH—.

In certain embodiments, X₂ represents hydrogen, alkyl, cycloalkyl, phenyl, 5- or 6-membered heterocycloalkyl or 5- or 6-membered heteroaryl. In certain embodiments of formula (IB), X₂ represents hydrogen.

In certain embodiments of formula (IB),
X₁ is C or N;
L represents (C₁-C₆)alkylene wherein one or more C atoms of (C₁-C₆)alkylene is replaced with N or O atom;
B represents —O— or —NH—;
B₁ and B₃ each independently represents —N— or —CX₂—; B₂ represents —N—;
X₂ represents hydrogen or alkyl;
R₁ at each occurrence independently represents alkyl, —NH₂, halo, haloalkyl, —CONH₂ or —OH; or
any two R₁ groups, bonded to adjacent carbon atoms, combine together to form a 5- or 6-membered heterocycloalkyl ring containing 1 or 2 heteroatoms selected from N, O and S; wherein the said heterocycloalkyl is substituted with one, two or three occurrences of R₃;
R₃ represents hydrogen, oxo, halo, hydroxyl, cycloalkyl, aryl or 5- or 6-membered heteroaryl;
R₄ is hydrogen, hydroxyl or halo;
R₅ₐ is 5- or 6-membered heteroaryl;
R_{5b} and R_{5c} each independently represents hydrogen, alkyl, acyl, ester, cycloalkyl, phenyl, aralkyl, 5- or 6-membered heterocycloalkyl or 5- or 6-membered heteroaryl;
‘n’ is an integer selected from 1, 2 and 3.

In certain embodiments, the present invention provides a compound of formula (IB) or a pharmaceutically acceptable salt or a stereoisomer or a prodrug thereof; wherein

X₁ is C or N;

R₁, at each occurrence, independently represents —CH₃, —F, —Cl, —CF₃, —CONH₂, —OH or —OCONHCH[CH(CH₃)₂]COOCH₃; or any two R₁ groups, bonded to adjacent carbon atoms, combine together to form

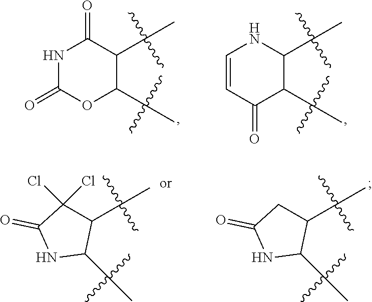 [SMILES: *C1OC(=O)NC(=O)C1* ; *C1NC=CC(=O)C1* ; *C1NC(=O)C(Cl)(Cl)C1* ; *C1CC(=O)NC1*]

R₄ represents hydrogen, hydroxyl, —F or —Cl;

L represents —CH₂—, —(CH₂—CH₂)—, —(CH₂—CH₂—CH₂)— or —(CH₂—CH₂—CH₂—CH₂)—;

B represents —O—, —NH— or —N(CH₃)—;

B₁, B₂ and B₃ independently represents —N— or —CH—;

R₅ₐ is furanyl, thienyl, pyrrolyl, pyrazolyl, imidazolyl, oxazolyl, isoxazolyl, thiazolyl, isothiazolyl, 1H-tetrazolyl, oxadiazolyl or triazolyl;

R_{5b} represents hydrogen, acyl or —COOCH₂CH(CH₃)₂;

R_{5c} is hydrogen;

‘n’ is an integer selected from 0, 1, 2 and 3.

In certain embodiments, the present invention provides compound of formula (IC),

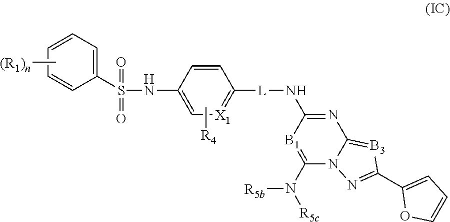

or a pharmaceutically acceptable salt or a stereoisomer or a prodrug thereof; and

X₁, R₁, R₄, L, B₁, B₃, R_{5b}, R_{5c} and ‘n’ are as defined in compound of formula (I).

In certain embodiments, X₂ represents hydrogen.

In certain embodiments, R₁ at each occurrence independently represents —NH₂, halo, —CONH₂, —OH or —OCONHCH[CH(CH₃)₂]COOCH₃. In certain embodiments of formula (IC), R₁, at each occurrence, independently represents —CH₃, —F, —Cl, —CF₃, —CONH₂, —OH or —OCONHCH[CH(CH₃)₂]COOCH₃.

In certain embodiments, any two R₁ groups, bonded to adjacent carbon atoms, combine together to form a 5- or 6-membered heterocycloalkyl ring containing 1 or 2 heteroatoms selected from N, O or S; wherein the said heterocycloalkyl is substituted with one, two or three occurrences of R₃; and R₃ represents hydrogen, oxo, halo or hydroxyl.

In certain embodiments, the present invention provides a compound of formula (IC) or a pharmaceutically acceptable salt or a stereoisomer or a prodrug thereof; wherein

X₁ is C or N;

R₁, at each occurrence, independently represents —CH₃, —F, —Cl, —CF₃, —CONH₂, —OH or —OCONHCH[CH(CH₃)₂]COOCH₃; or any two R₁ groups, bonded to adjacent carbon atoms, combine together to form

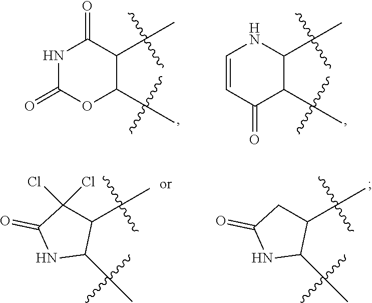 [SMILES: *C1OC(=O)NC(=O)C1* ; *C1NC=CC(=O)C1* ; *C1NC(=O)C(Cl)(Cl)C1* ; *C1CC(=O)NC1*]

R₄ represents hydrogen, hydroxyl, —F or —Cl;

L represents —CH₂—, —(CH₂—CH₂)—, —(CH₂—CH₂—CH₂)— or —(CH₂—CH₂—CH₂—CH₂)—;

B represents —O—, —NH— or —N(CH₃)—;

B₁ and B₃ independently represents —N— or —CH—;

R_{5b} represents hydrogen, acyl or —COOCH₂CH(CH₃)₂;

R_{5c} is hydrogen;

‘n’ is an integer selected from 0, 1, 2 and 3.

In certain embodiments, the present invention provides a compound of formula (ID),

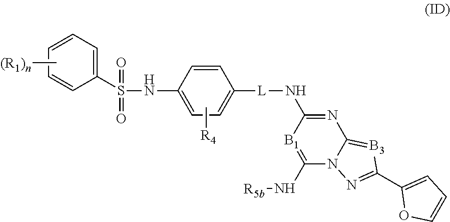

or a pharmaceutically acceptable salt or a stereoisomer or a prodrug thereof; and

R₁, R₄, L, B₁, B₃, R_{5b} and ‘n’ are as defined in compound of formula (I).

In certain embodiments of formula (ID), B₃ represents —N— or —CH—.

In certain embodiments of formula (ID), R_{5b} represents hydrogen, acyl, —COOCH₂CH(CH₃)₂.

In certain embodiments of formula (ID), L represents —CH₂—, —(CH₂—CH₂)—, —(CH₂—CH₂—CH₂)— or —(CH₂—CH₂—CH₂—CH₂)—.

In certain embodiments, the present invention provides a compound of formula (ID) or a pharmaceutically acceptable salt or a stereoisomer or a prodrug thereof; wherein

R₁, at each occurrence, independently represents —CH₃, —F, —Cl, —CF₃, —CONH₂, —OH or —OCONHCH[CH(CH₃)₂]COOCH₃; or any two R₁ groups, bonded to adjacent carbon atoms, combine together to form

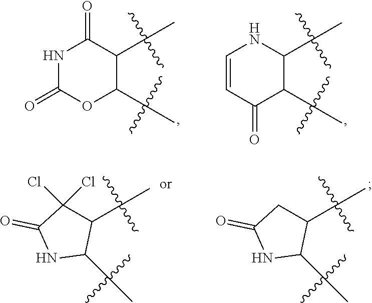 [SMILES: *C1OC(=O)NC(=O)C1* ; *C1NC=CC(=O)C1* ; *C1NC(=O)C(Cl)(Cl)C1* ; *C1CC(=O)NC1*]

R₄ represents hydrogen, hydroxyl, —F or —Cl;

L represents —CH₂—, —(CH₂—CH₂)—, —(CH₂—CH₂—CH₂)— or —(CH₂—CH₂—CH₂—CH₂)—;

B₁ and B₃ independently represents —N— or —CH—;

R_{5b} represents hydrogen, acyl or —COOCH₂CH(CH₃)₂;

‘n’ is an integer selected from 0, 1, 2 and 3.

In certain embodiments, the present invention provides compound of formula (IE),

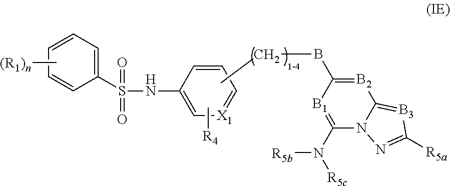

or a pharmaceutically acceptable salt or a stereoisomer or a prodrug thereof; wherein,

X₁, R₁, R₄, B, B₁, B₂, B₃, R₅ₐ, R_{5b}, R_{5c} and ‘n’ are as defined in compound of formula (I).

In certain embodiments of formula (IE), (—CH₂—)₁₋₄ represents —CH₂—, —(CH₂—CH₂)—, —(CH₂—CH₂—CH₂)— or —(CH₂—CH₂—CH₂—CH₂)—.

In certain embodiments of formula (IE), B₁, B₂ and B₃ independently represents —N— or —CH—In certain embodiments of formula (IE), R_{5b} represents hydrogen, acyl or —COOCH₂CH(CH₃)₂; and R_{5c} is hydrogen.

In certain embodiments, the present invention provides a compound of formula (IE) or a pharmaceutically acceptable salt or a stereoisomer or a prodrug thereof; wherein

X₁ is C or N;

R₁, at each occurrence, independently represents —CH₃, —F, —Cl, —CF₃, —CONH₂, —OH or —OCONHCH[CH(CH₃)₂]COOCH₃; or any two R₁ groups, bonded to adjacent carbon atoms, combine together to form

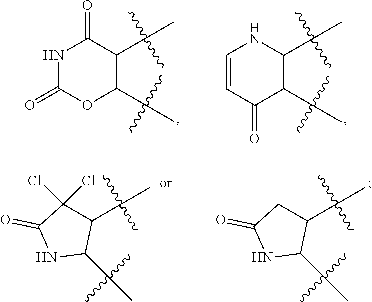 [SMILES: *C1OC(=O)NC(=O)C1* ; *C1NC=CC(=O)C1* ; *C1NC(=O)C(Cl)(Cl)C1* ; *C1CC(=O)NC1*]

R₄ represents hydrogen, hydroxyl, —F or —Cl;

B represents —O—, —NH— or —N(CH₃)—;

B₁, B₂ and B₃ independently represents —N— or —CH—;

R₅ₐ is furanyl, thienyl, pyrrolyl, pyrazolyl, imidazolyl, oxazolyl, isoxazolyl, thiazolyl, isothiazolyl, 1H-tetrazolyl, oxadiazolyl or triazolyl;

R_{5b} represents hydrogen, acyl or —COOCH₂CH(CH₃)₂;

R_{5c} is hydrogen;

‘n’ is an integer selected from 0, 1, 2 and 3.

In certain embodiments, the present invention provides compound of formula (IF),

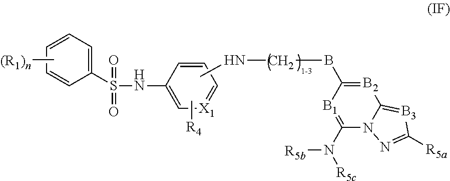

or a pharmaceutically acceptable salt or a stereoisomer or a prodrug thereof; wherein,

X₁, R₁, R₄, B, B₁, B₂, B₃, R₅ₐ, R_{5b}, R_{5c} and ‘n’ are as defined in compound of formula (I).

In certain embodiments, B represents —NR_{5d}—; wherein R_{5d} represents hydrogen or alkyl.

In certain embodiments, B₁ represents N. In certain embodiments, B₂ represents N or CH. In certain embodiments, B₂ represents N. In certain embodiments, B₃ represents N.

In certain embodiments, X₂ represents hydrogen or alkyl.

In certain embodiments, R_{5b} represents hydrogen, alkyl, acyl or ester. In certain embodiments, R_{5c} represents hydrogen.

In additional embodiments, R_{5d} represents hydrogen or alkyl.

In certain embodiments of formula (IE), R_{5b} represents hydrogen, acyl or —COOCH₂CH(CH₃)₂; and R_{5c} is hydrogen.

In certain embodiments, the present invention provides a compound of formula (IF) or a pharmaceutically acceptable salt or a stereoisomer or a prodrug thereof; wherein

X₁ is C or N;

R₁, at each occurrence, independently represents —CH₃, —F, —Cl, —CF₃, —CONH₂, —OH or —OCONHCH[CH(CH₃)₂]COOCH₃; or any two R₁ groups, bonded to adjacent carbon atoms, combine together to form

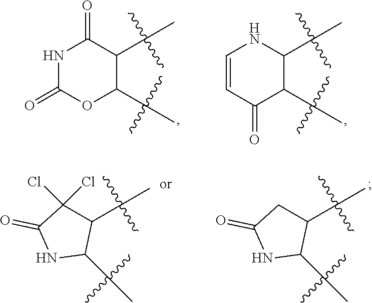 [SMILES: *C1OC(=O)NC(=O)C1* ; *C1NC=CC(=O)C1* ; *C1NC(=O)C(Cl)(Cl)C1* ; *C1CC(=O)NC1*]

R₄ represents hydrogen, hydroxyl, —F or —Cl;

B represents —O—, —NH— or —N(CH₃)—;

B₁, B₂ and B₃ independently represents —N— or —CH—;

R₅ₐ is furanyl, thienyl, pyrrolyl, pyrazolyl, imidazolyl, oxazolyl, isoxazolyl, thiazolyl, isothiazolyl, 1H-tetrazolyl, oxadiazolyl or triazolyl;

R_{5b} represents hydrogen, acyl or —COOCH₂CH(CH₃)₂;

R_{5c} is hydrogen;

‘n’ is an integer selected from 0, 1, 2 and 3.

In certain particular embodiments, the group of compound of formula (I) represented by

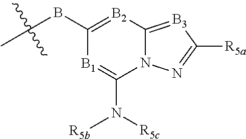

wherein [symbol] represents point of attachment to compound of formula (I) represents:

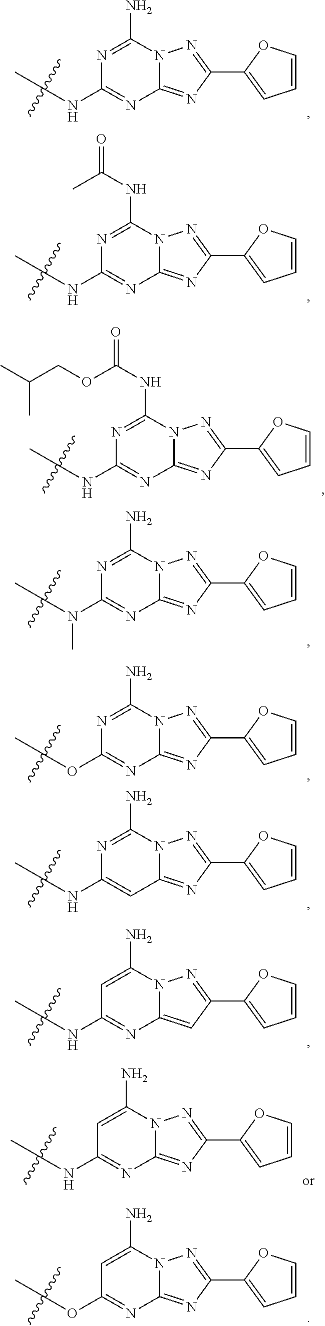

In certain particular embodiments,

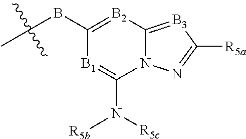

comprises:

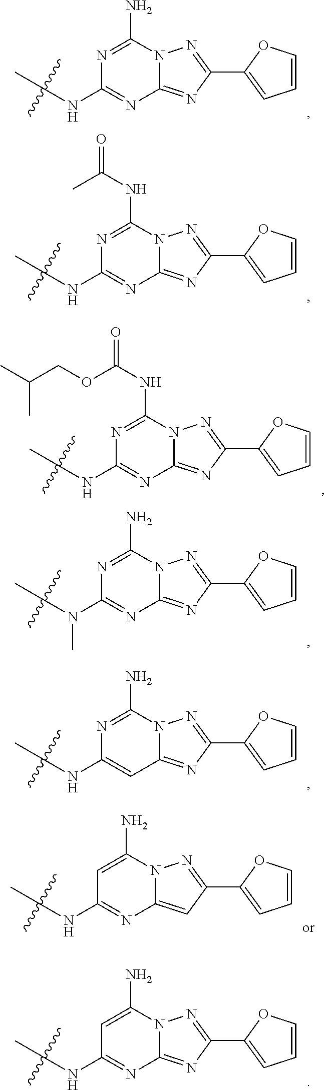

In certain embodiments,

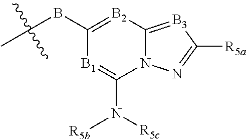

comprises:

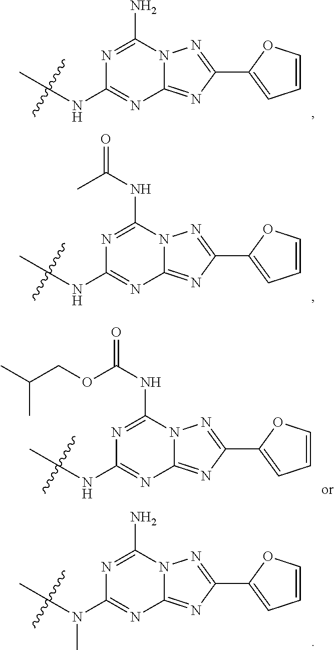

In certain particular embodiments,

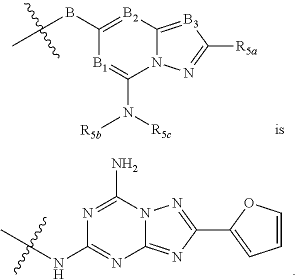

In certain embodiments of formula (I), wherein R₁, at each occurrence, independently represents —CH₃, —F, —Cl, —CF₃, —CONH₂, —OH or —OCONHCH[CH(CH₃)₂]COOCH₃.

In certain embodiments, any two R₁ groups, bonded to adjacent carbon atoms, combine together to form a 5- or 6-membered heterocycloalkyl ring containing 1 or 2 heteroatoms selected from N and O; wherein the said heterocycloalkyl is substituted with oxo.

In some embodiments, R₄ represents hydrogen, halo or alkyl. In some particular embodiments, R₄ represents hydrogen, halo. In certain embodiments, R₄ represents hydrogen.

In certain embodiments of compound of formula (I), L represents (C₁-C₆)alkylene, (C₂-C₆)alkenylene or (C₂-C₆)alkynylene, each (C₁-C₆)alkylene, (C₂-C₆)alkenylene or (C₂-C₆)alkynylene, is optionally substituted with one, two or three substituents selected from halo, hydroxyl, haloalkyl, amino, amido, (C₁-C₆)alkyl, aryl, cycloalkyl, heteroaryl and heterocycloalkyl.

In certain embodiments, L represents (C₁-C₃)alkylene, (C₂-C₃)alkenylene or (C₂-C₃)alkynylene, each (C₁-C₃)alkylene, (C₂-C₃)alkenylene or (C₂-C₃)alkynylene, is optionally substituted with one, two or three substituents selected from halo, hydroxyl, haloalkyl, amino, amido, (C₁-C₃)alkyl, aryl, cycloalkyl, heteroaryl and heterocycloalkyl.

In certain embodiments, L represents (C₁-C₃)alkylene or (C₂-C₃)alkynylene, each (C₁-C₃)alkylene or (C₂-C₃)alkynylene, is optionally substituted with substituents selected from halo, hydroxyl and (C₁-C₃)alkyl.

In certain embodiments of formula (I), wherein R₁ represents cycloalkyl, aryl, heteroaryl or heterocycloalkyl; wherein each of cycloalkyl, aryl, heteroaryl and heterocycloalkyl is substituted with one, two or three occurrences of R₃; wherein R₃ is hydrogen, oxo, halo, amino, alkyl, amido, hydroxyl, cycloalkyl, aryl, heteroaryl or heterocycloalkyl.

In certain embodiments, n is an integer selected from 0, 1, 2, 3 and 4. In certain embodiments, n is an integer selected from 0, 1, 2 and 3. In certain embodiments, n is an integer selected from 1, 2 and 3.

In certain embodiments, the present invention provides a compound of formula (I) or a pharmaceutically acceptable salt or a stereoisomer or a prodrug thereof; for the treatment of diseases or disorders mediated by CD73 and Adenosine receptors (A2aR and/or A2bR) in a subject.

In certain embodiments, the present invention provides a compound selected from:

| Example | IUPAC name |
|---|---|
| A1 | 5-(N-(3-(((7-amino-2-(furan-2-yl)-[1,2,4]triazolo[1,5-a][1,3,5]triazin-5- |
| | yl)amino)methyl)phenyl)sulfamoyl)-3-chloro-2-hydroxybenzamide; |
| A2 | 5-(N-(4-(((7-amino-2-(furan-2-yl)-[1,2,4]triazolo[1,5-a][1,3,5]triazin-5- |
| | yl)amino)methyl)phenyl)sulfamoyl)-3-chloro-2-hydroxybenzamide; |
| A3 | 5-(N-(4-(2-((7-amino-2-(furan-2-yl)-[1,2,4]triazolo[1,5-a][1,3,5]triazin-5- |
| | yl)amino)ethyl)phenyl)sulfamoyl)-3-chloro-2-hydroxybenzamide; |
| A4 | N-(4-(2-((7-amino-2-(furan-2-yl)-[1,2,4]triazolo[1,5-a][1,3,5]triazin-5- |
| | yl)amino)ethyl)phenyl)-3-chloro-5-fluoro-4-hydroxybenzenesulfonamide; |
| A5 | 5-(N-(4-(2-((7-amino-2-(furan-2-yl)-[1,2,4]triazolo[1,5-a][1,3,5]triazin-5- |
| | yl)amino)ethyl)-3-hydroxyphenyl)sulfamoyl)-3-chloro-2-hydroxybenzamide; |
| A6 | 5-(N-(4-(2-((7-amino-2-(furan-2-yl)-[1,2,4]triazolo[1,5-a][1,3,5]triazin-5- |
| | yl)amino)ethyl)-3-fluorophenyl)sulfamoyl)-3-chloro-2-hydroxybenzamide; |
| A7 | 5-(N-(4-(3-((7-amino-2-(furan-2-yl)-[1,2,4]triazolo[1,5-a][1,3,5]triazin-5- |
| | yl)amino)ethyl)-2-fluorophenyl)sulfamoyl)-3-chloro-2-hydroxybenzamide; |
| A8 | 5-(N-(4-(2-((7-acetamido-2-(furan-2-yl)-[1,2,4]triazolo[1,5-a][1,3,5]triazin-5- |
| | yl)amino)ethyl)phenyl)sulfamoyl)-3-chloro-2-hydroxybenzamide; |
| A9 | isobutyl (5-((4-((3-carbamoyl-5-chloro-4- |
| | hydroxyphenyl)sulfonamido)phenethyl)amino)-2-(furan-2-yl)- |
| | [1,2,4]triazolo[1,5-a][1,3,5]triazin-7-yl)carbamate; |
| A10 | N-(4-(2-((7-amino-2-(furan-2-yl)-[1,2,4]triazolo[1,5-a][1,3,5]triazin-5- |
| | yl)amino)ethyl)phenyl)-4-hydroxy-3-(trifluoromethyl)benzenesulfonamide; |
| A11 | 5-(N-(4-(3-((7-amino-2-(furan-2-yl)-[1,2,4]triazolo[1,5-a][1,3,5]triazin-5- |
| | yl)amino)propyl)phenyl)sulfamoyl)-3-chloro-2-hydroxybenzamide; |
| A12 | 5-(N-(4-(3-((7-amino-2-(furan-2-yl)-[1,2,4]triazolo[1,5-a][1,3,5]triazin-5- |
| | yl)oxy)propyl)phenyl)sulfamoyl)-3-chloro-2-hydroxybenzamide; |
| A13 | 5-(N-(4-(3-((7-amino-2-(furan-2-yl)-[1,2,4]triazolo[1,5-a][1,3,5]triazin-5- |
| | yl)amino)propyl)-3-hydroxyphenyl)sulfamoyl)-3-chloro-2-hydroxybenzamide; |
| A14 | 5-(N-(4-(3-((7-amino-2-(furan-2-yl)-[1,2,4]triazolo[1,5-a][1,3,5]triazin-5- |
| | yl)amino)propyl)-3-fluorophenyl)sulfamoyl)-3-chloro-2-hydroxybenzamide; |
| A15 | 5-(N-(4-(3-((7-amino-2-(furan-2-yl)-[1,2,4]triazolo[1,5-a][1,3,5]triazin-5- |
| | yl)amino)propyl)-2-fluorophenyl)sulfamoyl)-3-chloro-2-hydroxybenzamide; |
| A16 | 5-(N-(4-(3-((7-amino-2-(furan-2-yl)-[1,2,4]triazolo[1,5-a][1,3,5]triazin-5- |
| | yl)amino)propyl)phenyl)sulfamoyl)-3-chloro-2-methoxybenzamide; |
| A17 | 5-(N-(4-(3-((7-amino-2-(furan-2-yl)-[1,2,4]triazolo[1,5-a][1,3,5]triazin-5- |
| | yl)(methyl)amino)propyl)phenyl)sulfamoyl)-3-chloro-2-hydroxybenzamide; |
| A18 | 5-(N-(4-(4-((7-amino-2-(furan-2-yl)-[1,2,4]triazolo[1,5-a][1,3,5]triazin-5- |
| | yl)amino)butyl)phenyl)sulfamoyl)-3-chloro-2-hydroxybenzamide; |
| A19 | 5-(N-(4-(3-((5-amino-2-(furan-2-yl)-[1,2,4]triazolo[1,5-c]pyrimidin-7- |
| | yl)amino)propyl)phenyl)sulfamoyl)-3-chloro-2-hydroxybenzamide; |
| A20 | 5-(N-(4-(3-((7-amino-2-(furan-2-yl)-[1,2,4]triazolo[1,5-a]pyrimidin-5- |
| | yl)amino)propyl)phenyl)sulfamoyl)-3-chloro-2-hydroxybenzamide; |
| A21 | 5-(N-(4-(3-((7-amino-2-(furan-2-yl)-[1,2,4]triazolo[1,5-a]pyrimidin-5- |
| | yl)oxy)propyl)phenyl)sulfamoyl)-3-chloro-2-hydroxybenzamide; |
| A22 | 5-(N-(4-(3-((7-amino-2-(furan-2-yl)-[1,2,4]triazolo[1,5-a]pyrimidin-5- |
| | yl)amino)propyl)-3-fluorophenyl)sulfamoyl)-3-chloro-2-hydroxybenzamide; |
| A23 | 5-(N-(4-(3-((7-amino-2-(furan-2-yl)-[1,2,4]triazolo[1,5-a]pyrimidin-5- |
| | yl)amino)propyl)-2-fluorophenyl)sulfamoyl)-3-chloro-2-hydroxybenzamide; |
| A24 | 5-(N-(4-(3-((7-amino-2-(furan-2-yl)pyrazolo[1,5-a]pyrimidin-5- |
| | yl)amino)propyl)phenyl)sulfamoyl)-3-chloro-2-hydroxybenzamide |
| A25 | Methyl ((4-(N-(4-(3-((7-amino-2-(furan-2-yl)-[1,2,4]triazolo[1,5-a][1,3,5]triazin-5- |
| | yl)amino)propyl)phenyl)sulfamoyl)-2-carbamoyl-6- |
| | chlorophenoxy)carbonyl)valinate; |
| A26 | 5-(N-(4-(3-((7-amino-2-(furan-2-yl)-[1,2,4]triazolo[1,5-a][1,3,5]triazin-5- |
| | yl)amino)propyl)phenyl)sulfamoyl)-3-chloro-2-hydroxybenzamide sodium salt; |
| A27 | Ethyl (5-((3-(4-((3-chloro-N-(ethoxycarbonyl)-5-((ethoxycarbonyl)carbamoyl)-4- |
| | ((ethoxycarbonyl)oxy)phenyl)sulfonamido)phenyl)propyl)(ethoxycarbonyl)amino)- |
| | 2-(furan-2-yl)-[1,2,4]triazolo[1,5-a][1,3,5]triazin-7-yl)(ethoxycarbonyl)carbamate; |
| A28 | Tert-Butyl (1-(5-(N-(4-(3-((7-amino-2-(furan-2-yl)-[1,2,4]triazolo[1,5- |
| | a][1,3,5]triazin-5-yl)amino)propyl)phenyl)sulfamoyl)-3-chloro-2- |
| | hydroxybenzamido)-3-methyl-1-oxobutan-2-yl)carbamate; |
| A29 | Hexyl (7-amino-2-(furan-2-yl)-[1,2,4]triazolo[1,5-a][1,3,5]triazin-5-yl)(3-(4-((3- |
| | carbamoyl-5-chloro-4-hydroxyphenyl)sulfonamido)phenyl)propyl)carbamate; |
| A30 | 3-(Hexadecyloxy)propyl (7-amino-2-(furan-2-yl)-[1,2,4]triazolo[1,5- |
| | a][1,3,5]triazin-5-yl)(3-(4-((3-carbamoyl-5-chloro-4- |
| | hydroxyphenyl)sulfonamido)phenyl)propyl)carbamate; |
| A31 | 1-(((7-Amino-2-(furan-2-yl)-[1,2,4]triazolo[1,5-a][1,3,5]triazin-5-yl)(3-(4-((3- |
| | carbamoyl-5-chloro-4- |
| | hydroxyphenyl)sulfonamido)phenyl)propyl)carbamoyl)oxy)ethyl 3- |
| | methylbutanoate; |
| B1 | N-(4-(2-((7-amino-2-(furan-2-yl)-[1,2,4]triazolo[1,5-a][1,3,5]triazin-5- |
| | yl)amino)ethyl)phenyl)-7-chloro-2-oxoindoline-5-sulfonamide; |
| B2 | N-(4-(2-((7-amino-2-(furan-2-yl)-[1,2,4]triazolo[1,5-a][1,3,5]triazin-5- |
| | yl)amino)ethyl)phenyl)-3,3,7-trichloro-2-oxoindoline-5-sulfonamide; |
| B3 | N-(4-(2-((7-amino-2-(furan-2-yl)-[1,2,4]triazolo[1,5-a][1,3,5]triazin-5- |
| | yl)amino)ethyl)phenyl)-8-chloro-4-oxo-1,4-dihydroquinoline-6-sulfonamide; |
| B4 | N-(4-(3-((7-amino-2-(furan-2-yl)-[1,2,4]triazolo[1,5-a][1,3,5]triazin-5- |
| | yl)amino)propyl)phenyl)-8-chloro-2,4-dioxo-3,4-dihydro-2H- |
| | benzo[e][1,3]oxazine-6-sulfonamide; |
| C1 | 5-(N-(4-((2-((7-amino-2-(furan-2-yl)-[1,2,4]triazolo[1,5-a][1,3,5]triazin-5- |
| | yl)amino)ethyl)amino)phenyl)sulfamoyl)-3-chloro-2-hydroxybenzamide; |
| C2 | 5-(N-(3-((2-((7-amino-2-(furan-2-yl)-[1,2,4]triazolo[1,5-a][1,3,5]triazin-5- |
| | yl)amino)ethyl)amino)phenyl)sulfamoyl)-3-chloro-2-hydroxybenzamide; |
| D1 | 5-(N-(1-(2-((7-amino-2-(furan-2-yl)-[1,2,4]triazolo[1,5-a][1,3,5]triazin-5- |
| | yl)amino)ethyl)-1H-indol-6-yl)sulfamoyl)-3-chloro-2-hydroxybenzamide; |
| D2 | 5-(N-(1-(2-((7-amino-2-(furan-2-yl)-[1,2,4]triazolo[1,5-a][1,3,5]triazin-5- |
| | yl)amino)ethyl)-1H-indazol-4-yl)sulfamoyl)-3-chloro-2-hydroxybenzamide; |
| E1 | 5-(N-(6-(2-((7-amino-2-(furan-2-yl)-[1,2,4]triazolo[1,5-a][1,3,5]triazin-5- |
| | yl)amino)ethyl)pyridin-3-yl)sulfamoyl)-3-chloro-2-hydroxybenzamide; and |
| E2 | 5-(N-(6-(2-((7-amino-2-(furan-2-yl)-[1,2,4]triazolo[1,5-a]pyrimidin-5- |
| | yl)amino)ethyl)pyridin-3-yl)sulfamoyl)-3-chloro-2-hydroxybenzamide; |

or a pharmaceutical acceptable salt or a stereoisomer or a prodrug thereof.

### Definitions

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present invention.

The singular forms “a”, “an” and “the” encompass plural references unless the context clearly indicates otherwise.

As used herein, the terms “optional” or “optionally” mean that the subsequently described event or circumstance may occur or may not occur and that the description includes instances where the event or circumstance occurs as well as instances in which it does not. For example, “optionally substituted alkyl” refers to the alkyl may be substituted as well as the event or circumstance where the alkyl is not substituted. As another instance, “optional heteroaryl” refers to heteroaryl may be present as well as the event or circumstance where the heteroaryl is not present.

The term “substituted” refers to moieties having substituents replacing hydrogen on one or more carbons of the backbone. It will be understood that “substitution” or “substituted with” includes the implicit proviso that such substitution is in accordance with permitted valence of the substituted atom and the substituent and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Unless specifically stated, substituents can include any substituents described herein, for example, a halogen, a hydroxyl, a carbonyl (such as a carboxyl, an alkoxycarbonyl, a formyl or an acyl), a thiocarbonyl (such as a thioester, a thioacetate or a thioformate), an alkoxy, an oxo, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heteroaryl a heterocycloalkyl, an aralkyl or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, the term “alkyl” refers to saturated aliphatic groups, including but not limited to C₁-C₁₀ straight-chain alkyl groups or C₃-C₁₀ branched-chain alkyl groups. Preferably, “alkyl” group refers to C₁-C₆ straight-chain alkyl groups or C₃-C₆ branched-chain alkyl groups. Most preferably, the “alkyl” group refers to C₁-C₄ straight-chain alkyl groups or C₃-C₈ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl and 4-octyl. The “alkyl” group may be optionally substituted.

As used herein, the term “acyl” refers to —CO—R wherein R is alkyl group as defined. In some embodiments, acyl contains (C₁-C₆)alkyl. Exemplary acyl groups include, but not limited to, formyl, acetyl, propanoyl, 2-methylpropanoyl, t-butylacetyl and butanoyl.

As used herein, the term “ester” refers to ROCO—, wherein R is alkyl group as defined. Exemplary ester groups include, but not limited to, methoxycarbonyl, ethoxycarbonyl, propoxycarbonyl, butoxycarbonyl and pentoxycarbonyl.

As used herein, the term “alkenylene” refers to a carbon chain which contains at least one carbon-carbon double bond and which may be linear or branched or combinations thereof. Preferably “alkenylene” refers to (C₂-C₆)alkenylene. Examples of “alkenylene” include, but not limited to, vinylene, allylene, isopropenylene, pentenylene, hexenylene, heptenylene, 1-propenylene, 2-butenylene and 2-methyl-2-butenylene.

As used herein, the term “alkylene” means divalent, straight or branched chain hydrocarbon moieties containing one or more than one carbon-carbon single bonds. Examples of “alkylene” include, but not limited to, —CH₂—, —CH₂—CH₂— and —CH(CH₃)—CH₂—.

As used herein, the term “alkynylene” means divalent, straight or branched chain hydrocarbon moieties containing at least one carbon-carbon triple bonds. Examples of “alkynylene” include, but not limited to, ethynylene, propynylene, butynylene, pentynylene and hexynylene.

As used herein, the term “halo” or “halogen” alone or in combination with other term(s) means fluorine, chlorine, bromine or iodine.

As used herein, the term “haloalkyl” means alkyl substituted with one or more halogen atoms, wherein the halo and alkyl groups are as described herein. The term “halo” is used herein interchangeably with the term “halogen” means F, Cl, Br or I. Examples of “haloalkyl” include but are not limited to fluoromethyl, difluoromethyl, chloromethyl, trifluoromethyl and 2,2,2-trifluoroethyl.

As used herein, the term “hydroxy” or “hydroxyl” alone or in combination with other term(s) means —OH.

As used herein, the term “oxo” refers to ═O group.

As used herein, “amino” refers to an —NH₂ group. As used herein, “amido” refers to an —CONH₂ group.

As used herein, the term “amidated C-terminus” refers to that the C-terminal of the amino acid in amide form.

As used herein the term “esterified C-terminus” refers to that the C-terminal of the amino acid in ester form.

As used herein the term “free C-terminus” refers to that the C-terminal of the amino acid in —CO₂H form.

As used herein the term “free N-terminus” refers to that the N-terminal of the amino acid in —NH₂ form.

As used herein the term “Boc-protected N-terminus” refers to that the N-terminal of the amino acid protected with tert-butoxycarbonyl protecting group (BOC group).

The term “stereoisomers” refers to any enantiomers, diastereoisomers or geometrical isomers of the compounds of formula (I), wherever they are chiral or when they bear one or more double bonds. When the compounds of the formula (I) and related formulae are chiral, they can exist in racemic or in optically active form. It should be understood that the invention encompasses all stereochemical isomeric forms, including diastereomeric, enantiomeric and epimeric forms, as well as d-Isomers and l-Isomers and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric Isomers. The present invention includes all cis, trans, syn, anti, entgegen (E) and zusammen (Z) Isomers as well as the appropriate mixtures thereof.

As used herein the term “cycloalkyl” alone or in combination with other term(s) means —C₃-C₁₀ saturated cyclic hydrocarbon ring. A cycloalkyl may be a single ring, which typically contains from 3 to 7 carbon ring atoms. Examples of single ring cycloalkyls include but are not limited to cyclopropyl, cyclobutyl, cyclopentyl, cyclohexyl and cycloheptyl. A cycloalkyl may alternatively be polycyclic or contain more than one ring. Examples of polycyclic cycloalkyls include bridged, fused and spirocyclic carbocyclyls.

As used herein, the term ‘heterocycloalkyl’ refers to a non-aromatic, saturated or partially saturated, monocyclic or polycyclic ring system of 3- to 15-member having at least one heteroatom or heterogroup selected from O, N, S, S(O), S(O)₂, NH and C(O) with the remaining ring atoms being independently selected from the group consisting of carbon, oxygen, nitrogen and sulfur. The term “heterocycloalkyl” also refers to the bridged bicyclic ring system having at least one heteroatom or heterogroup selected from O, N, S, S(O), S(O)₂, NH and C(O). Examples of “heterocycloalkyl” include, but are not limited to azetidinyl, oxetanyl, imidazolidinyl, pyrrolidinyl, oxazolidinyl, thiazolidinyl, pyrazolidinyl, tetrahydrofuranyl, piperidinyl, piperazinyl, oxazinanyl, 1,3-oxazinanyl, tetrahydropyranyl, morpholinyl, thiomorpholinyl, 1,4-dioxanyl, dioxidothiomorpholinyl, oxapiperazinyl, oxapiperidinyl, tetrahydrofuryl, tetrahydropyranyl, tetrahydrothiophenyl, dihydropyranyl, indolinyl, indolinylmethyl, aza-bicyclooctanyl, azocinyl, chromanyl, xanthenyl and N-oxides thereof. Attachment of a heterocycloalkyl substituent can occur via either a carbon atom or a heteroatom. A heterocycloalkyl group can be optionally substituted with one or more suitable groups by one or more aforesaid groups. Preferably “heterocycloalkyl” refers to 5- to 6-membered ring selected from the group consisting of azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl and N-oxides thereof. All heterocycloalkyl are optionally substituted by one or more aforesaid groups.

As used herein, the term “heteroaryl” refers to an aromatic heterocyclic ring system containing, unless specifically stated, 5 to 20 ring atoms, suitably 5 to 10 ring atoms, which may be a single ring (monocyclic) or multiple rings (bicyclic, tricyclic, polycyclic or spirocyclic) fused together or linked covalently. Preferably, “heteroaryl” is a 5- to 6-membered ring. The rings may contain from 1 to 4 heteroatoms selected from N, O and S, wherein the N or S atom is optionally oxidized or the N atom is optionally quarternized. Any suitable ring position of the heteroaryl moiety may be covalently linked to the defined chemical structure.

Examples of heteroaryl include, but are not limited to: furanyl, thienyl, pyrrolyl, pyrazolyl, imidazolyl, oxazolyl, cinnolinyl, isoxazolyl, thiazolyl, isothiazolyl, 1H-tetrazolyl, oxadiazolyl, triazolyl, pyridyl, pyrimidinyl, pyrazinyl, pyridazinyl, benzoxazolyl, benzisoxazolyl, benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, α-carboline, indolizinyl, benzoisothiazolyl, benzoxazolyl, pyrrolopyridyl, pyrazolopyrimidyl, furopyridinyl, purinyl, benzothiadiazolyl, benzooxadiazolyl, benzotriazolyl, benzotriadiazolyl, carbazolyl, dibenzothienyl, acridinyl and the like. Preferably “heteroaryl” refers to 5- to 6-membered ring selected from the group consisting of furanyl, thienyl, pyrrolyl, pyrazolyl, imidazolyl, oxazolyl, cinnolinyl, isoxazolyl, thiazolyl, isothiazolyl, 1H-tetrazolyl, oxadiazolyl, triazolyl, pyridyl, pyrimidinyl, pyrazinyl and pyridazinyl. More preferably, pyrazolyl, pyridyl, oxazolyl and furanyl. All heteroaryls are optionally substituted by one or more aforesaid groups.

As used herein, the term ‘heteroaralkyl’ refers to a group wherein the ‘alkyl’ group is substituted with one or more ‘heteroaryl’ groups.

As used herein, the term “aryl” is optionally substituted monocyclic, bicyclic or polycyclic aromatic hydrocarbon ring system of about 6 to 14 carbon atoms. Examples of a C₆-C₁₄ aryl group include, but are not limited to, phenyl, naphthyl, biphenyl, anthryl, fluorenyl, indanyl, biphenylenyl and acenaphthyl. Aryl group can be unsubstituted or substituted with one or more suitable groups.

As used herein, the term ‘aralkyl’ refers to a group wherein the ‘alkyl’ group is substituted with one or more ‘aryl’ groups.

An “amino acid residue” as used herein, means a moiety sharing structural similarity to the parent amino acid. An amino acid residue may be covalently bonded to another chemical moiety via the amino group of the residue or the carboxylate group of the residue (i.e., a hydrogen atom of —NH₂ or —OH is replaced by a bond to another chemical moiety).

The term “heteroatom” as used herein designates a sulfur, nitrogen or oxygen atom.

As used herein, the term compound(s)′ comprises the compounds disclosed in the present invention.

As used herein, the term “comprise” or “comprising” is generally used in the sense of include, that is to say permitting the presence of one or more features or components.

As used herein, the term “or” means “and/or” unless stated otherwise.

As used herein, the term “including” as well as other forms, such as “include”, “includes” and “included” is not limiting.

As used herein, the term “composition” is intended to encompass a product comprising the specified ingredients in the specified amounts, as well as any product which results, directly or indirectly, from combination of the specified ingredients in the specified amounts.

The compounds described herein (e.g., compound wherein Rₐ is not hydrogen) may be prodrugs. The term “prodrug” when referring to a prodrug described herein (e.g. dual CD73 and A2aR inhibitor compound moiety bonded to a prodrug moiety) refers to the compound including dual CD73 and A2aR inhibitor compound moiety and the prodrug moiety. A “prodrug moiety” is the portion of a prodrug that may be cleaved from the prodrug resulting in an increased activity of the non-prodrug moiety portion of the prodrug, for example dual CD73 and A2aR inhibitor compound having increased activity relative to the prodrug thereof. In certain embodiments, the compounds described herein are prodrugs, wherein the prodrug moiety is released from the dual CD73 and A2aR inhibitor compound moiety upon degradation of the prodrug.

In certain embodiments, degradation of the prodrug includes cleavage of —ORₐ, wherein Rₐ is not hydrogen. In certain embodiments, degradation of the prodrug includes cleavage of —Rₐ wherein Rₐ is not hydrogen. A person having ordinary skill in the art would understand that the dual CD73 and A2aR inhibitor compound moiety includes only those compounds compatible with the chemistry provided herein for connecting the dual CD73 and A2aR inhibitor compound moiety to the prodrug moiety and for release of dual CD73 and A2aR inhibitor compound from its prodrug (e.g., in vivo). In embodiments, degradation of the prodrug releases an active agent (e.g., dual CD73 and A2aR inhibitor). In such compounds, the resulting active agent includes a higher level of activity compared to the level of activity of the intact prodrug.

As used herein, the term “pharmaceutical composition” refers to a composition(s) containing a therapeutically effective amount of at least one compound of formula (I) or (IA) or (IB) or a pharmaceutically acceptable salt or a stereoisomer or a prodrug thereof; and a pharmaceutically acceptable carrier.

The pharmaceutical composition(s) usually contain(s) about 1% to 99%, for example, about 5% to 75% or from about 10% to about 30% by weight of the compound of formula (I) or (II) or pharmaceutically acceptable salt or a stereoisomer or a prodrug thereof. The amount of the compound of formula (I) or pharmaceutically acceptable salt or a stereoisomer or a prodrug thereof in the pharmaceutical composition(s) can range from about 1 mg to about 1000 mg or from about 2.5 mg to about 500 mg or from about 5 mg to about 250 mg or in any range falling within the broader range of 1 mg to 1000 mg or higher or lower than the afore mentioned range.

As used herein, the term “treat”, “treating” and “treatment” refer to a method of alleviating or abrogating a disease and/or its attendant symptoms.

As used herein, the term “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

As used herein, the term “subject” refers to an animal, preferably a mammal and most preferably a human.

As used herein, the term, “therapeutically effective amount” refers to an amount of a compound of formula (I) or a pharmaceutically acceptable salt or a stereoisomer or a prodrug thereof; or a composition comprising the compound of formula (I) or a pharmaceutically acceptable salt or a stereoisomer or a prodrug thereof, effective in producing the desired therapeutic response in a particular patient suffering from a disease or disorder mediated by CD73 and/or Adenosine receptors. Particularly, the term “therapeutically effective amount” includes the amount of the compound of formula (I) or a pharmaceutically acceptable salt or a stereoisomer or a prodrug thereof, when administered, that induces a positive modification in the disease or disorder to be treated or is sufficient to prevent development of or alleviate to some extent, one or more of the symptoms of the disease or disorder being treated in a subject. In respect of the therapeutic amount of the compound, the amount of the compound used for the treatment of a subject is low enough to avoid undue or severe side effects, within the scope of sound medical judgment can also be considered. The therapeutically effective amount of the compound or composition will be varied with the particular condition being treated, the severity of the condition being treated or prevented, the duration of the treatment, the nature of concurrent therapy, the age and physical condition of the end user, the specific compound or composition employed the particular pharmaceutically acceptable carrier utilized.

“Pharmaceutically acceptable” means that, which is useful in preparing a pharmaceutical composition that is generally safe, non-toxic and neither biologically nor otherwise undesirable and includes that which is acceptable for veterinary as well as human pharmaceutical use.

“Pharmaceutically acceptable salt” refers to a product obtained by reaction of the compound of the present invention with a suitable acid or a base. Pharmaceutically acceptable salt of the compounds of this invention include those derived from suitable inorganic bases such as Li, Na, K, Ca, Mg, Fe, Cu, Al, Zn and Mn salts; Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention (compound of formula (I)) can form pharmaceutically acceptable salt with various organic bases such as lysine, arginine, guanidine, diethanolamine or metformin. Suitable base salts include, but are not limited to, aluminum, calcium, lithium, magnesium, potassium, sodium or zinc salts. In certain embodiments, pharmaceutically acceptable salts include the ones derived from inorganic bases Li, Na, K, Ca, Mg, Fe, Cu, Al, Zn and Mn salts. In certain embodiments, pharmaceutically acceptable salts include Na, K, Ca and Mg salts.

The present invention also provides methods for formulating the disclosed compounds as for pharmaceutical administration.

In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free or substantially pyrogen-free. The excipients can be chosen, for example, to effect delayed release of an agent or to selectively target one or more cells, tissues organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

In certain embodiments, present invention provides a pharmaceutical composition comprising the compound of formula (I) and a pharmaceutically acceptable salt or a stereoisomer or a prodrug thereof.

In certain embodiments, present invention provides pharmaceutical composition for use in treating and/or preventing a disease and/or disorder associated with CD73 and/or Adenosine receptors (particularly A2aR or A2bR).

In certain embodiments, the diseases or disorders dependent on CD73 and/or A2aR, include cancer.

In further embodiments, diseases or disorders dependent on CD73-A2aR/A2bR, are cancers, including, but not limited to brain gliomas, glioblastomas, astrocytomas, multiforme, bannayan-Zonana syndrome, Cowden disease, Lhermitte-Duclos disease, breast cancer, colon cancer, head and neck cancer, kidney, liver, lung cancer, bone cancer, colorectal cancer, germ cell cancer, melanoma, ovarian cancer, pancreatic cancer, adenocarcinoma, ductal adenocarcinoma, adenosquamous carcinoma, acinar cell carcinoma, glucagonoma, insulinoma, prostate, sarcoma and thyroid cancer, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, immunoblastic large cell leukemia, mantle cell leukemia, multiple myeloma, megakaryoblastic leukemia, multiple myeloma, acute megakaryocytic leukemia, promyelocytic leukemia, erythroleukemia, malignant lymphoma, Hodgkin's lymphoma, non-Hodgkin's lymphoma, lymphoblastic T cell lymphoma, Burkitt's lymphoma, follicular lymphoma, neuroblastoma, bladder cancer, urothelial cancer, vulval cancer, uterine/cervical cancer, endometrial cancer, renal cancer, mesothelioma, esophageal cancer, salivary gland cancer, hepatocellular cancer, gastric cancer, nasopharyngeal cancer, buccal cancer, cancer of the mouth, GIST (gastrointestinal stromal tumor), neuroendocrine cancers, testicular cancer or virus-related cancer. In certain embodiments, the cancer may be a metastatic cancer, refractory cancer or recurrent cancer.

In certain embodiments, the present invention provides use of the compounds as disclosed in the present invention for the preparation of a medicament for the treatment of cancer, more preferably for treating non-small cell lung cancer.

In certain embodiments, the present invention provides use of the compounds as disclosed in the present invention in the treatment of diseases or disorder associated with CD73 and/or Adenosine receptors.

In certain embodiments, the present invention provides use of the compound or a pharmaceutically acceptable salt or a stereoisomer or a prodrug thereof, for treating a disease in which the symptoms thereof are treated, improved, diminished and/or prevented by inhibition of CD73 and/or Adenosine receptors.

In certain embodiments, the compounds of the present invention are capable of selectively binding to and/or modulating CD73 and/or Adenosine receptors.

In certain embodiments, the compounds of the present invention are expected to be useful in the therapy of proliferative diseases such as cancers, including but not limited to carcinoma, including that of the breast, liver, lung, colon, kidney, bladder, including small cell lung cancer, non-small cell lung cancer, head and neck, thyroid, esophagus, stomach, pancreas, ovary, gall bladder, cervix, prostate and skin, including squamous cell carcinoma.

In certain embodiments, the compounds of the present invention can be administered in the form of a pharmaceutical composition to a patient in need of treatment of haematological malignancies which include but not limited to leukemias and lymphomas which include but not limited to hematopoietic tumors of lymphoid lineage, acute lymphoblastic leukemia, acute lymphocytic leukemia, Hodgkins lymphoma, non-Hodgkins lymphoma, B-cell lymphoma, T-cell lymphoma, hairy cell lymphoma, myeloma, mantle cell lymphoma and Burkett's lymphoma, hematopoietic tumors of myeloid lineage, including acute and chronic myelogenous leukemias, myelodysplastic syndrome and promyelocytic leukemia.

The compounds of the present invention may be used as single drug or as a pharmaceutical composition in which the compound is mixed with various pharmacologically acceptable materials.

The compounds of the invention are typically administered in the form of a pharmaceutical composition. Such compositions can be prepared using procedures well known in the pharmaceutical art and comprise at least one compound of this invention. The pharmaceutical composition of the present patent application comprises one or more compounds described herein and one or more pharmaceutically acceptable excipients. Typically, the pharmaceutically acceptable excipients are approved by regulatory authorities or are generally regarded as safe for human or animal use. The pharmaceutically acceptable excipients include, but are not limited to, carriers, diluents, glidants and lubricants, preservatives, buffering agents, chelating agents, polymers, gelling agents, viscosifying agents and solvents.

The pharmaceutical composition can be administered by oral, parenteral or inhalation routes. Examples of the parenteral administration include administration by injection, percutaneous, transmucosal, transnasal and transpulmonary administrations.

Examples of suitable carriers include, but are not limited to, water, salt solutions, alcohols, polyethylene glycols, peanut oil, olive oil, gelatin, lactose, terra alba, sucrose, dextrin, magnesium carbonate, sugar, amylose, magnesium stearate, talc, gelatin, agar, pectin, acacia, stearic acid, lower alkyl ethers of cellulose, silicic acid, fatty acids, fatty acid amines, fatty acid monoglycerides and diglycerides, fatty acid esters and polyoxyethylene.

The pharmaceutical composition may also include one or more pharmaceutically acceptable auxiliary agents, wetting agents, suspending agents, preserving agents, buffers, sweetening agents, flavouring agents, colorants or any combination of the foregoing.

The pharmaceutical compositions may be in conventional forms, for example, tablets, capsules, solutions, suspensions, injectables or products for topical application. Further, the pharmaceutical composition of the present invention may be formulated so as to provide desired release profile.

Administration of the compounds of the invention, in pure form or in an appropriate pharmaceutical composition, can be carried out using any of the accepted routes of administration of pharmaceutical compositions. The route of administration may be any route which effectively transports the active compound of the patent application to the appropriate or desired site of action. Suitable routes of administration include, but are not limited to oral, nasal, buccal, dermal, intradermal, transdermal, parenteral, rectal, subcutaneous, intravenous, intraurethral, intramuscular or topical.

Solid oral formulations include, but are not limited to, tablets, capsules (soft or hard gelatin), dragees (containing the active ingredient in powder or pellet form), troches and lozenges.

Liquid formulations include, but are not limited to, syrups, emulsions and sterile injectable liquids, such as suspensions or solutions.

Topical dosage forms of the compounds include ointments, pastes, creams, lotions, powders, solutions, eye or ear drops, impregnated dressings and may contain appropriate conventional additives such as preservatives, solvents to assist drug penetration.

The pharmaceutical compositions of the present patent application may be prepared by conventional techniques known in literature.

In certain embodiments, the present invention provides a composition comprising a compound of the disclosure and an excipient and/or pharmaceutically acceptable carrier for treating diseases or conditions or disorders that are dependent upon CD73 and adenosine receptor.

Suitable doses of the compounds for use in treating the diseases or disorders described herein can be determined by those skilled in the relevant art. Therapeutic doses are generally identified through a dose ranging study in humans based on preliminary evidence derived from the animal studies. Doses must be sufficient to result in a desired therapeutic benefit without causing unwanted side effects. Mode of administration, dosage forms and suitable pharmaceutical excipients can also be well used and adjusted by those skilled in the art. All changes and modifications are envisioned within the scope of the present patent application.

In certain embodiments, the disease or condition is treatable by adenosenergic pathway for example cancers, including, but not limited to brain gliomas, glioblastomas, astrocytomas, multiforme, bannayan-Zonana syndrome, Cowden disease, Lhermitte-Duclos disease, breast cancer, colon cancer, head and neck cancer, kidney, liver, lung cancer, melanoma, ovarian cancer, pancreatic cancer, adenocarcinoma, ductal adenocarcinoma, adenosquamous carcinoma, acinar cell carcinoma, glucagonoma, insulinoma, prostate, sarcoma and thyroid cancer, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, immunoblastic large cell leukemia, mantle cell leukemia, multiple myeloma, megakaryoblastic leukemia, multiple myeloma, acute megakaryocytic leukemia, promyelocytic leukemia, erythroleukemia, malignant lymphoma, Hodgkin's lymphoma, non-Hodgkin's lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharyngeal cancer, buccal cancer, cancer of the mouth, GIST (gastrointestinal stromal tumor), neuroendocrine cancers and testicular cancer.

According to one embodiment, the compounds of the present invention can also contain unnatural proportions of atomic isotopes at one or more of the atoms that constitute such compounds. For example, the present invention also embraces isotopically-labeled variants of the present invention which are identical to those recited herein, but for the fact that one or more atoms of the compound are replaced by an atom having the atomic mass or mass number different from the predominant atomic mass or mass number usually found in nature for the atom. All isotopes of any particular atom or element as specified are contemplated within the scope of the compounds of the invention and their uses. Exemplary isotopes that can be incorporated in to compounds of the invention include isotopes of hydrogen, carbon, nitrogen, oxygen, phosphorous, sulfur, fluorine, chlorine and iodine such as ²H (“D”), ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically labeled compounds of the present inventions can generally be prepared by following procedures analogous to those disclosed in the schemes and/or in the examples herein below, by substituting an isotopically labeled reagent for a non-isotopically labeled reagent.

The following abbreviations refer respectively to the definitions herein: BMS—Borane dimethylsulfide; CsF—Cesium Fluoride; DMSO—Dimethylsulfoxide; DIPEA—N,N-Diisopropylethylamine; NaHCO₃—Sodium bicarbonate; EDC.HCl—EtOH—Ethanol; EtOAc—Ethyl acetate; Dioxane.HCl—Hydrochloric acid in dioxane; Na₂SO₄—Sodium sulphate; H₂O—water; br—Broad; ° C.—Degree Celsius; DMAP—4-Dimethylaminopyridine; DMSO-d₆—Deuterated dimethylsulfoxide; CH₂Cl₂—DCM—Dichloromethane; DMF—N,N-Dimethylformamide; g—Gram; h—Hours; ¹H—Proton; HCl—Hydrochloric acid; Hz—Hertz; J—Coupling Constant; K₂CO₃—Potassium carbonate, LCMS—Liquid Chromatography—Mass Spectroscopy; HPLC—High-performance liquid chromatography; LiCl—Lithium chloride; LiOH—Lithium hydroxide; L-Boc valine—Boc protected L-valine; MeOH—methanol; MeONa—Sodium methoxide; MeCN—Methyl Cyanide; MeI—Methyl Iodide; M—Molar; MHz—Mega Hertz (frequency); MS—Mass Spectroscopy; mmol—Milli Mole; mL—Milli Litre; min—Minutes; mol—Moles; Mt Molecular ion; m/z—mass to charge ratio; N—Normality; NaH—Sodium Hydride; NMR—Nuclear Magnetic Resonance; Et₃N/TEA—Triethyl amine; ppm—Parts per million; rt/RT—Room temperature; s—Singlet; d—Doublet, t—TBAB—Tetrabutylammonium bromide; Triplet; q—Quartet; m—Multiplet; dd—doublet of doublets; td—triplet of doublets; qd—quartet of doublets; ddd—doublet of doublet of doublets; dt—doublet of triplets; ddt—doublet of doublet of triplets; p-pentet; Pd(OAC)₂—Palladium(II) acetate; TBAB—Tetra-n-butylammonium bromide; TLC—Thin Layer Chromatography; THF—Tetrahydrofuran; %—Percentage; μ—Micron; μL-Micro liter; μM-Micro molar; and δ—Delta.

### General Modes of Preparation:

Following general guidelines apply to all experimental procedures described here. Until otherwise stated, experiments are performed under positive pressure of nitrogen, temperature describes are the external temperature (i.e. oil bath temperature). Reagents and solvents received from vendors are used as such without any further drying or purification. Molarities mentioned here for reagents in solutions are approximate as it was not verified by a prior titration with a standard. All reactions are stirred under magnetic stir bar. Cooling to minus temperature was done by acetone/dry ice or wet ice/salts. Magnesium sulfate and sodium sulfate were used as solvent drying agent after reaction work up and are interchangeable. Removing of solvents under reduced pressure or in vacuo or concentration of the reaction mixture means distilling of solvents in rotary evaporator.

Compounds of this invention may be made by synthetic chemical processes, examples of which are shown herein. It is meant to be understood that the order of the steps in the processes may be varied, that reagents, solvents and reaction conditions may be substituted for those specifically mentioned and that vulnerable moieties may be protected and deprotected, as necessary.

The specifics of the process for preparing compounds of the present invention are detailed in the experimental section.

The present invention shall be illustrated by means of some examples, which are not construed to be viewed as limiting the scope of the invention.

### EXPERIMENTAL

Unless otherwise stated, work-up includes distribution of the reaction mixture between the organic and aqueous phases, separation of layers and drying the organic layer over anhydrous sodium sulphate, filtration and evaporation of the solvent. Purification, unless otherwise mentioned, includes purification by silica gel chromatographic techniques, generally using ethyl acetate/petroleum ether mixture of a suitable polarity as the mobile phase.

Analysis for the compounds of the present invention unless mentioned, was conducted in the general methods well known to the person skilled in the art. Having described the invention with reference to certain preferred embodiments, other embodiments will become apparent to one skilled in the art from consideration of the specification. The invention is further defined by reference to the following examples, describing in detail the analysis of the compounds of the invention.

It will be apparent to those skilled in the art that many modifications, both to materials and methods, may be practiced without departing from the scope of the invention. Some of the intermediates were taken to next step based on TLC results, without further characterization, unless otherwise specified.

The MS (Mass Spectral) data provided in the examples were obtained using the equipment(s)-API 2000 LC/MS/MS/Triplequad; Agilent Technologies/LC/MS/DVL/Singlequad; Shimadzu LCMS-2020/Singlequad.

The NMR data provided in the examples were obtained using the equipment(s)—¹H-NMR: Varian 400 MHz and Varian 300 MHz.

The HPLC performed for the provided examples using equipment-Agilent Technologies 1200 Series; Agilent Technologies 1100 Series; Shimadzu (UFLC) Prominance; Shimadzu Nexera-UHPLC.

Compound purifications were performed on CombiFlashx unless otherwise mentioned.

### General Procedure for the Preparation of Examples A1 to A4

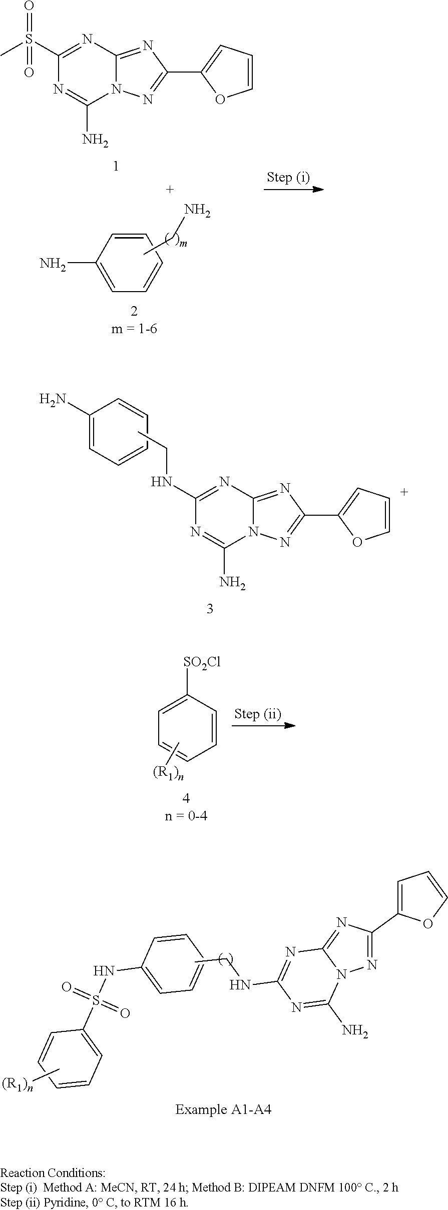

### Step (i): General Procedure for the Synthesis of Intermediate 3

The compound 2-(furan-2-yl)-5-(methylsulfonyl)-[1,2,4]triazolo[1,5-a][1,3,5]triazin-7-amine (intermediate 1) was prepared from furoic hydrazide by following the procedure described in Journal of Medicinal Chemistry, 58(2), 718-738; 2015.

Method A: To a stirred solution of 2-(furan-2-yl)-5-(methylsulfonyl)-[1,2,4]triazolo[1,5-a][1,3,5]triazin-7-amine (Intermediate 1) in MeCN, a relevant amine (2) was added. The mixture was allowed to stir for 24 h at RT under nitrogen atmosphere. Upon completion of the reaction as indicated by TLC, the reaction mixture was concentrated under vacuum to provide the crude. It was then purified by combi-flash silica-gel chromatography.

Method B: To a stirred solution of 2-(furan-2-yl)-5-(methylsulfonyl)-[1,2,4]triazolo[1,5-a][1,3,5]triazin-7-amine (Intermediate 1) in DMF, relevant amine (2) and DIPEA was added. The mixture was heated at 100° C. and stirred for 2 h at RT under nitrogen atmosphere. Upon completion of the reaction as indicated by TLC, the reaction mixture was quenched with ice-water and extracted with EtOAc three times. The combined organic layer was washed with brine, dried over Na₂SO₄ and volatiles were removed under vacuum to provide the crude. It was used as such for the next step without further purification.

### Synthesis of N⁵-(3-aminobenzyl)-2-(furan-2-yl)-[1,2,4]triazolo[1,5-a][1,3,5]triazine-5,7-diamine (3a)

The title compound was prepared by a reaction of intermediate 1 (350 mg) and 3-(aminomethyl)aniline (2a, 330 mg, 2.70 mmol) by following the general procedure described in step (i): Method A. The crude was purified by combi-flash silica-gel chromatography using 4% MeOH/DCM as eluent to obtain the title compound as white solid (170 mg, 43%). LCMS: m/z 323.1 (M+H⁺); ¹H NMR (400 MHz, DMSO-d₆): δ8.21 (bs, 2H), 7.85-7.83 (m, 2H), 7.03-6.91 (m, 2H), 6.66 (d, J=1.6 Hz, 1H), 6.50-6.39 (m, 3H), 5.00 (s, 2H), 4.39-4.35 (m, 2H) ppm.

### Synthesis of N⁵-(4-aminobenzyl)-2-(furan-2-yl)-[1,2,4]triazolo[1,5-a][1,3,5]triazine-5,7-diamine (3b)

The title compound was prepared by a reaction of intermediate 1 (600 mg) and 4-(aminomethyl)aniline (2b, 522 mg, 4.28 mmol) by following the general procedure described in step (i): Method B as off-white solid (300 mg, 43%). LCMS: m/z 323.2 (M+H⁺); ¹H NMR (400 MHz, DMSO-d₆): δ 8.04 (bs, 2H), 7.85-7.75 (m, 2H), 7.04-7.03 (m, 1H), 6.99 (d, J=8.4 Hz, 2H), 6.67-6.66 (m, 1H), 6.49 (d, J=8.4 Hz, 2H), 4.91 (s, 2H), 4.31-4.29 (m, 2H) ppm.

### Synthesis of N⁵-(4-aminophenethyl)-2-(furan-2-yl)-[1,2,4]triazolo[1,5-a][1,3,5]triazine-5,7-diamine (3c)

The title compound was prepared by a reaction of intermediate 1 (300 mg) and 4-(2-aminoethyl)aniline (2c, 291 mg, 2.13 mmol) by following the general procedure described in step (i): Method B as brown solid (170 mg, 43%). LCMS: m/z 337.05 (M+H⁺); ¹H NMR (400 MHz, DMSO-d₆): δ 8.15 (bs, 2H), 7.86 (s, 1H), 7.43-7.40 (m, 1H), 7.05 (d, J=3.6 Hz, 1H), 6.89 (d, J=8.0 Hz, 2H), 6.67 (d, J=1.6 Hz, 1H), 6.50 (d, J=8.4 Hz, 2H), 4.86 (s, 2H), 3.40-3.36 (m, 2H), 2.67-2.63 (m, 2H) ppm.

### Synthesis of Intermediates 4a & 4b

### 3-carbamoyl-5-chloro-4-hydroxybenzenesulfonyl chloride (Intermediate 4a)

The title compound was prepared by a reaction of 3-chloro-2-hydroxybenzamide (7 g, 40.9 mmol) with thionyl chloride (4 ml) and chlorosulphonic acid (8 ml) according to the procedures described in International publication WO2017153952. It was used for next reaction without further purification. Yield: 1.5 g, 50%; LC-MS: m/z 269.85 (M⁺).

### 3-chloro-5-fluoro-4-hydroxybenzenesulfonyl chloride (Intermediate 4b)

The title compound was prepared by a reaction of 2-chloro-6-fluorophenol (1.8 g, 12.3 mmol) with thionyl chloride (1 ml) and chlorosulphonic acid (2 ml) according to the procedures described in International publication WO2017153952. It was used for next reaction without further purification. Yield: 7.5 g, 68%; LC-MS: m/z 243.0 (M−H⁺).

### Step (ii): Preparation of Example A1

### Example A1: 5-(N-(3-(((7-amino-2-(furan-2-yl)-[1,2,4]triazolo[1,5-a][1,3,5]triazin-5-yl)amino)methyl)phenyl)sulfamoyl)-3-chloro-2-hydroxybenzamide

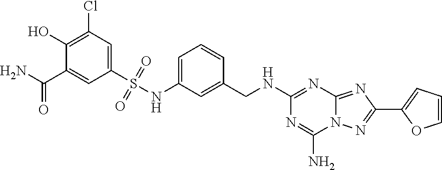 [SMILES: NC(=O)c1cc(S(=O)(=O)Nc2cccc(CNc3nc(N)n4nc(-c5ccco5)nc4n3)c2)cc(Cl)c1O]

To a stirred solution of intermediate (3a) (130 mg) in pyridine, 3-carbamoyl-5-chloro-4-hydroxybenzenesulfonyl chloride (4a, 120 mg, 0.44 mmol) was added at 0° C. The resultant mixture was slowly warmed to RT and stirred for 16 h. Upon completion of the reaction as indicated by TLC, the reaction mixture was concentrated to provide the crude compound. The crude was purified by combi-flash silica-gel chromatography followed by preparative HPLC to obtain the title compound as white solid (30 mg, 15%).

LCMS: m/z 556.1 (M⁺); HPLC: 99.39%.

¹H NMR (400 MHz, DMSO-d₆): δ 15.04 (bs, 1H), 10.14 (s, 1H), 9.14 (bs, 1H), 8.33-7.90 (m, 5H), 7.85 (s, 2H), 7.18-7.13 (m, 2H), 7.08-6.94 (m, 3H), 6.66 (s, 1H), 4.44-4.40 (m, 2H) ppm.

### Example A2: 5-(N-(4-(((7-amino-2-(furan-2-yl)-[1,2,4]triazolo[1,5-a][1,3,5]triazin-5-yl)amino)methyl)phenyl)sulfamoyl)-3-chloro-2-hydroxybenzamide

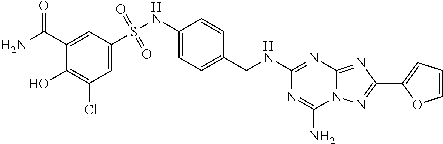 [SMILES: NC(=O)c1cc(S(=O)(=O)Nc2ccc(CNc3nc(N)n4nc(-c5ccco5)nc4n3)cc2)cc(Cl)c1O]

The title compound was prepared by a reaction of intermediate 3b (100 mg) and 3-carbamoyl-5-chloro-4-hydroxybenzenesulfonyl chloride (4a, 110 mg, 0.41 mmol) by following the general procedure described in step (ii) of Example A1 as white solid (15 mg, 7%). LCMS: m/z 556.1 (M⁺); HPLC: 95.88% ¹H NMR (400 MHz, DMSO-d₆): δ 15.00 (bs, 1H), 10.11 (s, 1H), 9.05 (bs, 1H), 8.43-8.31 (m, 2H), 8.15 (bs, 2H), 7.87-7.85 (m, 3H), 7.20 (d, J=8.4 Hz, 2H), 7.06-7.02 (m, 3H), 6.66 (dd, J=3.6 Hz, 2.0 Hz, 1H), 4.39-4.38 (m, 2H) ppm.

### Example A3: 5-(N-(4-(2-((7-amino-2-(furan-2-yl)-[1,2,4]triazolo[1,5-a][1,3,5]triazin-5-yl)amino)ethyl)phenyl)sulfamoyl)-3-chloro-2-hydroxybenzamide

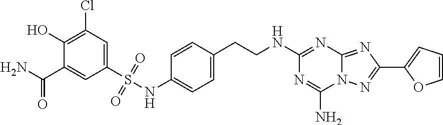 [SMILES: NC(=O)c1cc(S(=O)(=O)Nc2ccc(CCNc3nc(N)n4nc(-c5ccco5)nc4n3)cc2)cc(Cl)c1O]

The title compound was prepared by a reaction of intermediate 3c (100 mg) and 3-carbamoyl-5-chloro-4-hydroxybenzenesulfonyl chloride (4a, 88.3 mg, 0.32 mmol) by following the general procedure described in step (ii) of Example A1 as white solid (45 mg, 28%). LCMS: m/z 570.0 (M⁺); HPLC: 99.30% ¹H NMR (400 MHz, DMSO-d₆): δ 15.05 (bs, 1H), 10.04 (s, 1H), 9.10 (bs, 1H), 8.29-8.13 (m, 4H), 7.85 (s, 2H), 7.50-7.40 (m, 1H), 7.13 (d, J=8.4 Hz, 2H), 7.05-7.02 (m, 3H), 6.67 (d, J=1.6 Hz, 1H), 3.41-3.30 (m, 2H), 2.77-2.67 (m, 2H) ppm.

### Example A4: N-(4-(2-((7-amino-2-(furan-2-yl)-[1,2,4]triazolo[1,5-a][1,3,5]triazin-5-yl)amino)ethyl)phenyl)-3-chloro-5-fluoro-4-hydroxybenzenesulfonamide

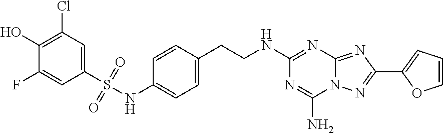 [SMILES: Nc1nc(NCCc2ccc(NS(=O)(=O)c3cc(F)c(O)c(Cl)c3)cc2)nc2nc(-c3ccco3)nn12]

The title compound was prepared by a reaction of intermediate 3c (150 mg) and 3-chloro-5-fluoro-4-hydroxybenzenesulfonyl chloride (4b, 117 mg, 0.48 mmol) by following the general procedure described in step (ii) of Example A1 as white solid (30 mg, 12%). LCMS: m/z 545.0 (M⁺); HPLC: 99.97%. ¹H NMR (400 MHz, DMSO-d₆): δ 12.73 (s, 1H), 11.68 (bs, 1H), 10.12 (s, 1H), 8.13 (s, 1H), 7.86 (s, 1H), 7.55-7.45 (m, 3H), 7.15-7.13 (m, 2H), 7.04-7.00 (m, 3H), 6.86-6.67 (m, 1H), 3.43-3.38 (m, 2H), 2.76-2.71 (m, 2H) ppm.

### Example A5: 5-(N-(4-(2-((7-amino-2-(furan-2-yl)-[1,2,4]triazolo[1,5-a][1,3,5]triazin-5-yl)amino)ethyl)-3-hydroxyphenyl)sulfamoyl)-3-chloro-2-hydroxybenzamide

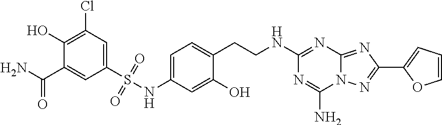 [SMILES: NC(=O)c1cc(S(=O)(=O)Nc2ccc(CCNc3nc(N)n4nc(-c5ccco5)nc4n3)c(O)c2)cc(Cl)c1O]

### Step (i): Synthesis of 2-(I-hydroxy-2-nitroethyl)-5-nitrophenol (6)

To a solution of nitromethane (0.48 mL, 8.982 mmol) in anhydrous MeOH (15 mL), MeONa (0.485 g, 8.982 mmol) was added at 0° C. and the solution was stirred at RT for 2 h. Then 2-hydroxy-4-nitrobenzaldehyde (5, 1 g, 5.988 mmol) was added at 0° C. and stirred at RT for 3 h.

Upon completion of the reaction (as indicated by TLC), the reaction mixture was quenched with ice-water (10 mL) and acidified with 2N HCl. It was extracted with EtOAc (3×100 mL). The combined organic extracts were washed with brine dried over anhydrous sodium sulfate to provide the crude which was purified with silica-gel chromatography using 15% EtOAc/hexane as eluent to obtain the title compound as red gel (1.0 g, 74%). LC-MS: m/z 227 (M−H+).

### Step (ii): Synthesis of (E)-5-nitro-2-(2-nitrovinyl)phenol (7)

To a solution of intermediate 6 (1 g, 4.385 mmol) in water (5 mL) at RT, tri-n-butyltin hydride (2 mL) was added at RT and the reaction mixture was heated at 80° C. for 30 min in microwave. The mixture was cooled to room temperature and extracted with EtOAc (3×10 mL). The combined organic extracts were washed with brine and dried over anhydrous sodium sulfate. The crude was purified with silica-gel chromatography using 12% EtOAc/hexane as eluent to obtain the title compound as light yellow solid (0.35 g, 63%). ¹H NMR (400 MHz, d₆-DMSO): δ 11.86 (bs, 1H), 8.27 (d, J=13.6 Hz, 1H), 8.17 (d, J=13.6 Hz, 1H), 7.98 (d, J=8 Hz, 1H) 7.72-7.68 (m, 2H) ppm.

### Step (iii): Synthesis of 5-amino-2-(2-aminoethyl)phenol (8)

To a stirred solution of 10% palladium on charcoal (0.2 g) in MeOH, a solution of (E)-5-nitro-2-(2-nitrovinyl)phenol (7, 0.75 g, 3.751 mmol) in MeOH was added. The resulting suspension was stirred at RT for 16 h under H₂ balloon (50 psi). Upon completion of the reaction (as indicated by TLC), the reaction mixture was filtered through Celite® bed. The Celite® bed was washed with MeOH (25 ml×2) and combined organic layer was dried under vacuum to obtain the title compound (0.160 g, 28%). LC-MS: m/z 153.1 (M+H⁺).

### Step (iv): Synthesis of 5-amino-2-(2-((7-amino-2-(furan-2-yl)-[1,2,4]triazolo[1,5-a][1,3,5]triazin-5-yl)amino)ethyl)phenol (9)

The title compound was prepared by a reaction of 2-(furan-2-yl)-5-(methylsulfonyl)-[1,2,4]triazolo[1,5-a][1,3,5]triazin-7-amine (1, 0.16 g) and 5-amino-2-(2-aminoethyl)phenol (8, 0.279 g, 1.05 mmol) by following the general procedure described in step (i): Method B of preparation of Example A1 to A4 (0.18 g, crude). LCMS: m/z 353.1 (M+H⁺).

### Step (v): Synthesis of 5-(N-(4-(2-((7-amino-2-(furan-2-yl)-[1,2,4]triazolo[1,5-a][1,3,5]triazin-5-yl)amino)ethyl)-3-hydroxyphenyl)sulfamoyl)-3-chloro-2-hydroxybenzamide (Example-A5)

The title compound was prepared by a reaction of intermediate 9 (0.1 g) and 3-carbamoyl-4-hydroxybenzenesulfonyl chloride (4a, 0.077 g, 0.28 mmol) by following the general procedure described in step (ii) of Example A1 as white solid (0.004 g, 1.3%). LCMS: m/z 586.1 (M+H⁺); HPLC: 99.07%; ¹H NMR (400 MHz, DMSO-d₆): δ 15.02 (s, 1H), 9.91 (bs, 1H), 9.52-9.48 (m, 1H), 8.23-8.03 (s, 4H), 7.86-7.73 (m, 2H), 7.46-7.36 (m, 1H), 7.05-6.92 (m, 2H), 6.68-6.49 (m, 4H), 3.40-3.37 (m, 2H), 2.68-2.66 (m, 2H) ppm.

### Preparation of Examples A6 & A7

### Step (i-a): Synthesis of ethyl 2-cyano-2-(2-fluoro-4-nitrophenyl)acetate (12a)

To a stirred solution of 1,2-difluoro-4-nitrobenzene (10a, 3 g) in MeCN and ethyl cyanoacetate (11, 2.55 g, 22.64 mmol) and K₂CO₃ were added at 0° C. The resulting mixture was heated at 90° C. for 16 h. Upon completion of the reaction (as indicated by TLC), the reaction mixture was quenched with ice-water and extracted with EtOAc. The combined organic layer was washed with brine, dried over Na₂SO₄ and volatiles were removed under vacuum to provide the crude title compound. It was then purified by combi-flash silica-gel chromatography using 0-20% EtOAc/hexane as eluent to obtain the title compound (4 g, 83%).

LC-MS: m/z 250.95 (M−H⁺); ¹H NMR (400 MHz, DMSO-d₆): δ 8.10-8.07 (m, 1H), 7.97 (dd, J=9.2 Hz, 2.4 Hz, 1H), 7.71-7.68 (m, 1H), 5.03 (s, 1H), 4.26-4.23 (m, 2H), 1.27-1.25 (m, 3H) ppm.

### Step (i-b): Synthesis of ethyl 2-cyano-2-(3-fluoro-4-nitrophenyl)acetate (12b)

The title compound was prepared by a reaction of 1,3-difluoro-4-nitrobenzene (10b, 3 g) and ethyl cyanoacetate (11, 2.55 g, 22.64 mmol) by following the procedure described in step (i-a): to obtain the intermediate 12b (4.5 g, 94%). LC-MS: m/z 252.2 (M+H⁺); ¹H NMR (400 MHz, DMSO-d₆): δ 8.32-8.28 (m, 1H), 7.51 (dd, J=8.8 Hz, 3.2 Hz, 1H), 7.33-7.28 (m, 1H), 5.69 (s, 1H), 4.32 (q, J=7.2 Hz, 2H), 1.32 (t, J=6.8 Hz, 1H) ppm.

### Step (ii-a): Synthesis of 2-(2-fluoro-4-nitrophenyl)acetonitrile (13a)

To a stirred solution of ethyl 2-cyano-2-(2-fluoro-4-nitrophenyl)acetate (12a, 4 g) in DMSO, NaCl (1.74 g, 31.74 mmol) was added at 0° C. The resulting mixture was heated at 100° C. for 12 h. Upon completion of the reaction (as indicated by TLC), the reaction mixture was quenched with ice-water and extracted with EtOAc. The combined organic layer was washed with brine, dried over Na₂SO₄ and volatiles were removed under vacuum to provide the crude compound. It was then purified by combi-flash silica-gel chromatography using 0-20% EtOAc/hexane as eluent to obtain title compound; (2.2 g, 78%). LC-MS: m/z 179.0 (M−H⁺).

### Step (ii-b): Synthesis of 2-(3-fluoro-4-nitrophenyl)acetonitrile (13b)

The title compound was prepared by a reaction of ethyl 2-cyano-2-(2-fluoro-4-nitrophenyl)acetate (12b, 4.5 g) and NaCl (1.97 g, 36 mmol) by following the general procedure described in step (ii-b) to obtain the title compound (2.2 g, crude). ¹H NMR (400 MHz, DMSO-d₆): δ 8.24-8.20 (m, 1H), 7.41 (dd, J=8.8 Hz, 2.4 Hz, 1H), 7.21-7.16 (m, 1H), 4.18 (s, 2H) ppm.

### Step (iii-a): Synthesis of 2-(2-fluoro-4-nitrophenyl)ethan-1-amine (14a)

To a stirred solution of intermediate 13a (1 g) in THF, 1M BMS in THE (27.8 ml, 27.8 mmol) was added at 0° C. The resulting mixture was heated at 80° C. for 2 h. Upon completion of the reaction (as indicated by TLC), the reaction mixture was quenched with ice-water and extracted with EtOAc three times. The combined organic layer was washed with brine, dried over Na₂SO₄ and volatiles were removed under vacuum to provide the crude compound. It was then purified by combi-flash silica-gel chromatography using 0-30% EtOAc/hexane as eluent to obtain the title compound (0.4 g, 40%). LC-MS: m/z 183.15 (M−H+).

### Step (iii-b): Synthesis of 2-(3-fluoro-4-nitrophenyl)ethan-1-amine (14b)

The title compound was prepared by a reaction of 2-(2-fluoro-4-nitrophenyl)acetonitrile (13b, 1.2 g) and 1M BMS in THE (33.3 ml, 33.3 mmol) by following the general procedure described in step (iii-a) to obtain the intermediate 14b (0.6 g, 49%). LC-MS: m/z 184.95 (M+H⁺).

### Step (iv-a): Synthesis of 4-(2-aminoethyl)-3-fluoroaniline (I5a)

The title compound was prepared by a reaction of 2-(2-fluoro-4-nitrophenyl)ethan-1-amine (14a, 0.4 g) and 10% palladium on charcoal (0.1 g) by following the procedure described in step (iii) of Example A5 (0.32 g, crude). LC-MS: m/z 155.15 (M+H⁺).

### Step (iv-b): Synthesis of 4-(2-aminoethyl)-2-fluoroaniline (15b)

The title compound was prepared by a reaction of 2-(3-fluoro-4-nitrophenyl)ethan-1-amine (14b, 0.6 g) and 10% palladium on charcoal (0.2 g) by following the general procedure described in step (iii) of Example A5. Yield: 0.25 g, crude. LC-MS: m/z 155.05 (M+H⁺).

### Step (v-a): Synthesis of N⁵-(4-amino-2-fluorophenethyl)-2-(furan-2-yl)-[1,2,4]triazolo[1,5-a][1,3,5]triazine-5,7-diamine (16a)

The title compound was prepared by a reaction of 2-(furan-2-yl)-5-(methylsulfonyl)-[1,2,4]triazolo[1,5-a][1,3,5]triazin-7-amine (Intermediate 1, 0.2 g) and intermediate 15a (0.16 g, 1.07 mmol) by following the general procedure described in step (i): Method B of preparation of Example A1 to A4. Yield: 0.180 g, crude. LCMS: m/z 355.0 (M+H⁺).

### Step (v-b): Synthesis of N⁵-(4-amino-3-fluorophenethyl)-2-(furan-2-yl)-[1,2,4]triazolo[1,5-a][1,3,5]triazine-5,7-diamine (16b)

The title compound was prepared by a reaction of 2-(furan-2-yl)-5-(methylsulfonyl)-[1,2,4]triazolo[1,5-a][1,3,5]triazin-7-amine (0.2 g) and intermediate 15b (0.16 g, 1.07 mmol) by following the general procedure described in step (i): Method B (0.120 g, crude) of preparation of Example A1 to A4. LCMS: m/z 355.1 (M+H⁺).

### Example A6: 5-(N-(4-(2-((7-amino-2-(furan-2-yl)-[1,2,4]triazolo[1,5-a][1,3,5]triazin-5-yl)amino)ethyl)-3-fluorophenyl)sulfamoyl)-3-chloro-2-hydroxybenzamide

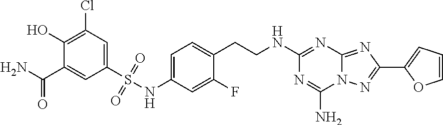 [SMILES: NC(=O)c1cc(S(=O)(=O)Nc2ccc(CCNc3nc(N)n4nc(-c5ccco5)nc4n3)c(F)c2)cc(Cl)c1O]

The title compound was prepared by a reaction of intermediate 16a (180 mg) and 3-carbamoyl-4-hydroxybenzenesulfonyl chloride (4a, 0.20 mg, 0.76 mmol) by following the general procedure described in step (ii) of Example A1 as white solid (0.025 g, 9%). LCMS: m/z 588.1 (M⁺); HPLC: 99.55%; ¹H NMR (400 MHz, DMSO-d₆): δ 15.09 (s, 1H), 10.32 (s, 1H), 9.19 (s, 1H), 8.39-8.13 (m, 4H), 7.86 (s, 2H), 7.52-7.44 (m, 1H), 7.20-7.16 (m, 1H), 7.05-7.04 (m, 1H), 6.92-6.85 (m, 2H), 6.67 (s, 1H), 3.42-3.40 (m, 2H), 2.78-2.76 (m, 2H) ppm.

### Example A7: 5-(N-(4-(3-((7-amino-2-(furan-2-yl)-[1,2,4]triazolo[1,5-a][1,3,5]triazin-5-yl)amino) ethyl)-2-fluorophenyl)sulfamoyl)-3-chloro-2-hydroxybenzamide

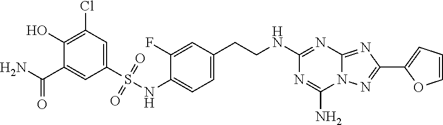 [SMILES: NC(=O)c1cc(S(=O)(=O)Nc2ccc(CCNc3nc(N)n4nc(-c5ccco5)nc4n3)cc2F)cc(Cl)c1O]

The title compound was prepared by a reaction of intermediate 16b (120 mg) and 3-carbamoyl-4-hydroxybenzenesulfonyl chloride (4a, 0.137 g, 0.5 mmol) by following the general procedure described in step (ii) of Example A1 as white solid (0.015 g, 8%). LCMS: m/z 587.95 (M⁺); HPLC: 98.17%; ¹H NMR (400 MHz, DMSO-d₆): δ 15.18 (s, 1H), 12.25 (s, 1H), 9.54-9.51 (m, 1H), 9.05 (bs, 1H), 8.37-8.36 (m, 2H), 7.89-7.87 (m, 2H), 7.60-7.51 (m, 1H), 7.19-7.17 (m, 1H), 7.06-6.96 (m, 2H), 6.72-6.67 (m, 2H), 3.46-3.43 (m, 2H), 2.92-2.89 (m, 2H) ppm.

### Example E1: 5-(N-(6-(2-((7-amino-2-(furan-2-yl)-[1,2,4]triazolo[1,5-a][1,3,5]triazin-5-yl)amino)ethyl)pyridin-3-yl)sulfamoyl)-3-chloro-2-hydroxybenzamide

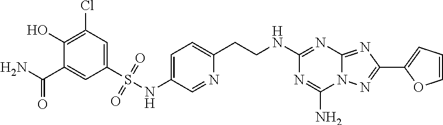 [SMILES: NC(=O)c1cc(S(=O)(=O)Nc2ccc(CCNc3nc(N)n4nc(-c5ccco5)nc4n3)nc2)cc(Cl)c1O]

### Step (i): Synthesis of N⁵-(2-(5-aminopyridin-2-yl)ethyl)-2-(furan-2-yl)-[1,2,4]triazolo[1,5-a][1,3,5]triazine-5,7-diamine (18)

The title compound was prepared by a reaction of 2-(furan-2-yl)-5-(methylsulfonyl)-[1,2,4]triazolo[1,5-a][1,3,5]triazin-7-amine (1, 0.2 g) and 6-(2-aminoethyl)pyridin-3-amine (intermediate 17) (0.14 g, 0.78 mmol) by following the general procedure described in step (i): Method B of preparation of Examples A1 to A4 (0.17 g, 71%). LCMS: m/z 338.1 (M+H⁺); ¹H NMR (400 MHz, DMSO-d₆): δ 8.13 (bs, 2H), 7.90-7.86 (m, 2H), 7.42-7.38 (m, 1H), 7.04-6.89 (m, 3H), 6.67 (s, 1H), 5.14 (s, 2H), 3.53-3.50 (m, 2H), 2.81-2.78 (m, 2H) ppm.

### Step (ii): Synthesis of 5-(N-(6-(2-((7-amino-2-(furan-2-yl)-[1,2,4]triazolo[1,5-a][1,3,5]triazin-5-yl)amino)ethyl)pyridin-3-yl)sulfamoyl)-3-chloro-2-hydroxybenzamide (Example E1)

The title compound was prepared by a reaction of intermediate 18 (0.17 g) and 3-carbamoyl-4-hydroxybenzenesulfonyl chloride (4a, 0.18 g, 0.65 mmol) by following the general procedure described in step (ii) of Example A1 as off-white solid (0.020 g, 7%). LCMS: m/z 571.1 (M⁺); HPLC: 99.90%; ¹H NMR (400 MHz, DMSO-d₆): δ 15.07 (s, 1H), 12.78 (s, 1H), 9.97 (s, 1H), 8.22-8.13 (m, 3H), 8.01-7.99 (m, 1H), 7.86-7.83 (m, 2H), 7.48-7.36 (m, 3H), 7.20-7.18 (m, 1H), 7.04 (s, 1H), 6.66 (s, 1H), 3.55-3.53 (m, 2H), 2.93-2.91 (m, 2H) ppm.

### Example A8: 5-(N-(4-(2-((7-acetamido-2-(furan-2-yl)-[1,2,4]triazolo[1,5-a][1,3,5]triazin-5-yl)amino)ethyl)phenyl)sulfamoyl)-3-chloro-2-hydroxybenzamide

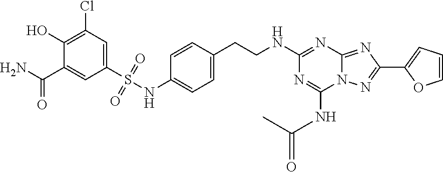 [SMILES: CC(=O)Nc1nc(NCCc2ccc(NS(=O)(=O)c3cc(Cl)c(O)c(C(N)=O)c3)cc2)nc2nc(-c3ccco3)nn12]

To a stirred solution of Example A3 (0.1 g) in THF, triethyl amine and acetyl chloride (19a, 14 mg, 0.175 mmol) were added at 0° C. The resulting mixture was slowly warmed to RT and stirred for 2 h. Upon completion of the reaction (as indicated by TLC), water was added to the reaction mixture and extracted with EtOAc for three times. The combined organic layer was washed with brine, dried over Na₂SO₄ and volatiles were removed under vacuum to provide the crude compound. It was then purified by preparative HPLC to obtain the title compound as white solid (0.013 g, 12%).

LCMS: m/z 612.05 (M⁺); HPLC: 99.33%; ¹H NMR (400 MHz, DMSO-d₆): δ 14.19 (s, 1H), 9.76 (s, 1H), 8.07 (d, J=2.8 Hz, 1H), 7.86 (s, 1H), 7.52 (d, J=2.8 Hz, 1H), 7.44-7.42 (m, 1H), 7.20 (s, 1H), 7.10-6.95 (m, 7H), 6.67 (s, 1H), 3.45-3.42 (m, 2H), 2.73-2.71 (m, 2H), 2.32 (m, 3H) ppm.

### Example A9: Isobutyl (5-((4-((3-carbamoyl-5-chloro-4-hydroxyphenyl)sulfonamido) phenethyl)amino)-2-(furan-2-yl)-[1,2,4]triazolo[1,5-a][1,3,5]triazin-7-yl)carbamate

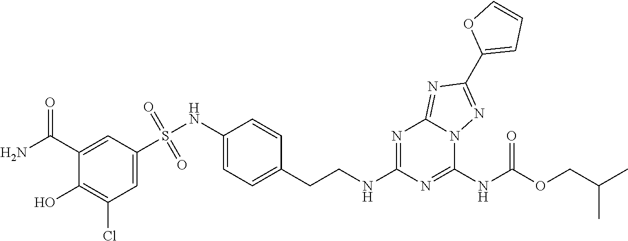 [SMILES: CC(C)COC(=O)Nc1nc(NCCc2ccc(NS(=O)(=O)c3cc(Cl)c(O)c(C(N)=O)c3)cc2)nc2nc(-c3ccco3)nn12]

The title compound was prepared by a reaction of Example A3 (0.12 g) and isobutylchloroformate (19b, 28.6 mg, 0.21 mmol) by following the procedure described in Example A8 as off-white solid (20 mg, 15%).

LCMS: m/z 670.1 (M⁺); HPLC: 97.25%; ¹H NMR (400 MHz, DMSO-d₆): δ 14.35 (s, 1H), 9.77 (s, 1H), 8.03 (d, J=2.8 Hz, 1H), 7.86 (s, 1H), 7.59-7.43 (m, 2H), 7.20-6.94 (m, 8H), 6.67 (s, 1H), 3.83 (d, J=6.8 Hz, 2H), 3.46-3.45 (m, 2H), 2.76-2.72 (m, 2H), 1.92-1.85 (m, 1H), 0.90 (d, J=6.8 Hz, 6H) ppm.

### Example A10: N-(4-(2-((7-amino-2-(furan-2-yl)-[1,2,4]triazolo[1,5-a][1,3,5]triazin-5-yl)amino)ethyl)phenyl)-4-hydroxy-3-(trifluoromethyl)benzenesulfonamide

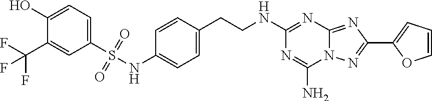 [SMILES: Nc1nc(NCCc2ccc(NS(=O)(=O)c3ccc(O)c(C(F)(F)F)c3)cc2)nc2nc(-c3ccco3)nn12]

The title compound was prepared by a reaction of intermediate 3c (0.3 g) and 4-hydroxy-3-(trifluoromethyl)benzenesulfonyl chloride (22, 0.38 g, 1.15 mmol) by following the procedure described in step (ii) of Example A1 as off-white solid (0.040 g, 6%). LCMS: m/z 561.00 (M+H⁺); HPLC: 99.89%; ¹H NMR (400 MHz, DMSO-d₆): δ 11.72 (s, 1H), 10.06 (s, 1H), 8.15 (bs, 2H), 7.87-7.50 (m, 4H), 7.13-6.98 (m, 6H), 6.69-6.67 (m, 1H), 6.67 (s, 1H), 3.41-3.39 (m, 2H), 2.77-2.73 (m, 2H) ppm.

### Preparation of Examples B1 & B2

### Step (i): Synthesis of 7-chloro-2-oxoindoline-5-sulfonyl chloride (21a)

To the mixture of HSO₃Cl (5 mL) and SOCl₂ (2.5 mL) was added 7-chloroindolin-2-one (intermediate 20a) (0.5 g, 2.99 mmol) at 0° C. The resulting suspension was stirred at RT for 2 h. Upon completion of the reaction (as indicated by TLC), the reaction mixture was quenched with water and extracted with EtOAc three times. The combined organic layer was washed with brine, dried over Na₂SO₄ and volatiles were removed under vacuum to obtain the title compound. It was used in next step without any further purification Yield: (0.7 g, 88%). LC-MS: m/z 266.0 (M⁺); ¹H NMR (400 MHz, DMSO-d₆): δ 0.80 (s, 1H), 7.38 (s, 1H), 7.35 (s, 1H), 3.73 (s, 2H) ppm.

### Step (ii): Synthesis of 3,3,7-trichloro-2-oxoindoline-5-sulfonyl chloride (21b)

The title compound was prepared by a reaction of ClSO₃H (5 mL) with 7-chloroindoline-2,3-dione (intermediate 20b) (1 g, 5.50 mmol) by following the general procedure described in step (i). Yield: 1.1 g, crude. LC-MS: m/z 333.8 (M+H⁺); ¹H NMR (400 MHz, DMSO-d₆): 11.96 (s, 1H), 7.70 (d, J=0.8 Hz, 1H), 7.12 (d, J=1.2 Hz, 1H) ppm.

### Example B1: N-(4-(2-((7-amino-2-(furan-2-yl)-[1,2,4]triazolo[1,5-a][1,3,5]triazin-5-yl)amino)ethyl)phenyl)-7-chloro-2-oxoindoline-5-sulfonamide

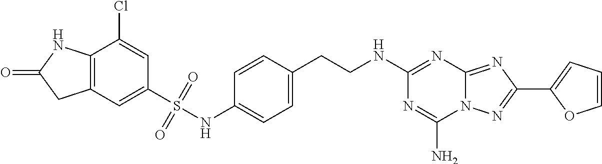 [SMILES: Nc1nc(NCCc2ccc(NS(=O)(=O)c3cc(Cl)c4c(c3)CC(=O)N4)cc2)nc2nc(-c3ccco3)nn12]

To a stirred solution of intermediate 3c (0.1 g, 0.297 mmol) in DCM (5 mL) and pyridine (1 mL), DMAP (3.6 mg, 0.029 mmol) and intermediate 21a (0.087 g, 0.327 mmol) were added. The reaction mixture was stirred at RT for 16 h. Upon completion of the reaction as indicated by TLC, the reaction mixture was quenched with water and extracted with EtOAc for three times. The combined organic layer was washed with brine, dried over Na₂SO₄ and volatiles were removed under vacuum. The crude was then purified by preparative HPLC to obtain the title compound as off-white solid (0.010 g, 6%).

LCMS: m/z 566.05 (M+); HPLC: 99.75%; ¹H NMR (400 MHz, DMSO-d₆): δ 11.08 (s, 1H), 10.04 (s, 1H), 8.36 (s, 1H), 8.09 (bs, 1H), 7.85 (s, 1H), 7.58 (s, 1H), 7.51-7.41 (m, 1H), 7.12-7.00 (m, 5H), 6.67 (s, 1H), 3.66 (s, 2H), 3.41-3.38 (m, 2H), 2.76-2.73 (m, 2H) ppm.

### Example B2: N-(4-(2-((7-amino-2-(furan-2-yl)-[1,2,4]triazolo[1,5-a][1,3,5]triazin-5-yl)amino)ethyl)phenyl)-3,3,7-trichloro-2-oxoindoline-5-sulfonamide

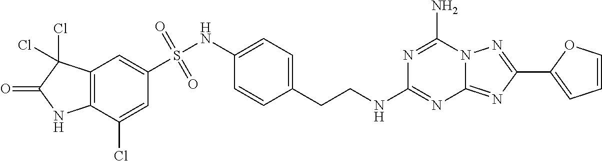 [SMILES: Nc1nc(NCCc2ccc(NS(=O)(=O)c3cc(Cl)c4c(c3)C(Cl)(Cl)C(=O)N4)cc2)nc2nc(-c3ccco3)nn12]

The title compound was prepared by a reaction of intermediate 3c (0.2 g) and 3,3,7-trichloro-2-oxoindoline-5-sulfonyl chloride (21b, 0.313 g, 0.93 mmol) by following the procedure described for Example B1 as white solid (0.020 g, 4%).

LCMS: m/z 635.90 (M+H⁺); HPLC: 99.80%; ¹H NMR (400 MHz, DMSO-d₆): δ 12.31 (s, 1H), 10.19 (s, 1H), 8.20-8.06 (m, 2H), 7.86 (s, 1H), 7.81 (s, 1H), 7.76 (d, J=2 Hz, 1H), 7.50-7.42 (m, 1H), 7.17-7.15 (m, 2H), 7.04-7.02 (m, 3H), 6.67 (s, 1H), 3.41-3.39 (m, 2H), 2.77-2.75 (m, 2H) ppm.

### Example B3: N-(4-(2-((7-amino-2-(furan-2-yl)-[1,2,4]triazolo[1,5-a][1,3,5]triazin-5-yl)amino)ethyl)phenyl)-8-chloro-4-oxo-1,4-dihydroquinoline-6-sulfonamide

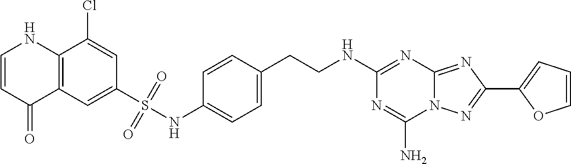 [SMILES: Nc1nc(NCCc2ccc(NS(=O)(=O)c3cc(Cl)c4[nH]ccc(=O)c4c3)cc2)nc2nc(-c3ccco3)nn12]

The title compound was prepared by a reaction of intermediate 3c (0.1 g) and 8-chloro-4-oxo-1,4-dihydroquinoline-6-sulfonyl chloride (23, 0.09, 0.33 mmol) by following the general procedure described in in step (ii) of Example A1, as white solid (0.003 g, 2%). LCMS: m/z 577.9 (M+H⁺)⁺; HPLC: 100%; ¹H NMR (400 MHz, DMSO-d₆): δ 8.31 (s, 1H), 8.13-8.10 (m, 1H), 8.01-7.99 (m, 1H), 7.85-7.83 (m, 2H), 7.48-7.34 (m, 2H), 7.07-7.03 (m, 4H), 6.66 (s, 1H), 6.55 (s, 1H), 6.50 (s, 1H), 3.47-3.43 (m, 2H), 2.43-2.42 (m, 2H) ppm.

### Preparation of Examples A11, & A12

### Step (i): Synthesis of N⁵-(3-(4-aminophenyl)propyl)-2-(furan-2-yl)-[1,2,4]triazolo[1,5-a][1,3,5]triazine-5,7-diamine (25a)

The title compound was prepared by a reaction of 2-(furan-2-yl)-5-(methylsulfonyl)-[1,2,4]triazolo[1,5-a][1,3,5]triazin-7-amine (1, 5 g) and 4-(3-aminopropyl)aniline (24a, 3.2 g, 21.33 mmol) by following the general procedure described in step (i): Method B of preparation of Examples A1 to A4 (3.5 g, 56.4%). LCMS: m/z 351.2 (M+H⁺); ¹H NMR (400 MHz, DMSO-d₆): δ 8.12 (bs, 2H), 7.86 (s, 1H), 7.54-7.45 (m, 1H), 7.05-7.04 (m, 1H), 6.86 (d, J=8 Hz, 2H), 6.67 (s, 1H), 6.48 (d, J=8 Hz, 2H), 4.83 (s, 2H), 3.26-3.21 (m, 2H), 2.46-2.44 (m, 2H), 1.76-1.72 (m, 2H) ppm.

### Example A11: 5-(N-(4-(3-((7-amino-2-(furan-2-yl)-[1,2,4]triazolo[1,5-a][1,3,5]triazin-5-yl)amino)propyl)phenyl)sulfamoyl)-3-chloro-2-hydroxybenzamide

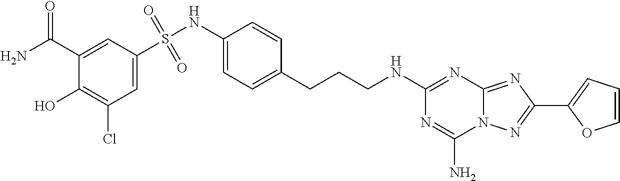 [SMILES: NC(=O)c1cc(S(=O)(=O)Nc2ccc(CCCNc3nc(N)n4nc(-c5ccco5)nc4n3)cc2)cc(Cl)c1O]

The title compound was prepared by a reaction of intermediate 25a (3.5 g) and 3-carbamoyl-4-hydroxybenzenesulfonyl chloride (4a, 2.84 g, 10.59 mmol) by following the general procedure described in step (ii) of Example A1 as white solid (1.0 g, 17.2%). LCMS: m/z 584.05 (M+H⁺); HPLC: 99.92%; ¹H NMR (400 MHz, DMSO-d₆): δ 15.05 (s, 1H), 12.72 (s, 1H), 9.97 (s, 1H), 8.24-8.13 (m, 2H), 7.86-7.77 (m, 2H), 7.54-7.44 (m, 1H), 7.11-6.99 (m, 5H), 6.68-6.66 (m, 1H), 6.54 (s, 1H), 3.27-3.20 (m, 2H), 2.54-2.52 (m, 2H), 1.78-1.71 (m, 2H) ppm.

### Step (i): Synthesis of 5-(3-(4-aminophenyl)propoxy)-2-(furan-2-yl)-[1,2,4]triazolo[1,5-a][1,3,5]triazin-7-amine (25b)

The title compound was prepared by a reaction of 2-(furan-2-yl)-5-(methylsulfonyl)-[1,2,4]triazolo[1,5-a][1,3,5]triazin-7-amine (1, 0.3 g) and 3-(4-aminophenyl)propan-1-ol (24b, 0.29 g, 2.13 mmol) by following the general procedure described in step (i): Method B of preparation of Examples A1 to A4 (0.17 g, 46%). LCMS: m/z 351.05 (M+H⁺); ¹H NMR (400 MHz, DMSO-d₆): δ 8.89 (s, 2H), 8.69 (s, 1H), 7.92-7.91 (m, 1H), 7.14-7.13 (m, 1H), 7.05-7.04 (m, 1H), 6.87 (d, J=8 Hz, 2H), 6.71-6.70 (m, 1H), 6.50-6.47 (m, 2H), 4.85 (s, 2H), 4.27-4.25 (m, 2H), 2.56-2.54 (m, 2H), 1.95-1.91 (m, 2H) ppm.

### Example A12: 5-(N-(4-(3-((7-amino-2-(furan-2-yl)-[1,2,4]triazolo[1,5-a][1,3,5]triazin-5-yl)oxy)propyl)phenyl)sulfamoyl)-3-chloro-2-hydroxybenzamide

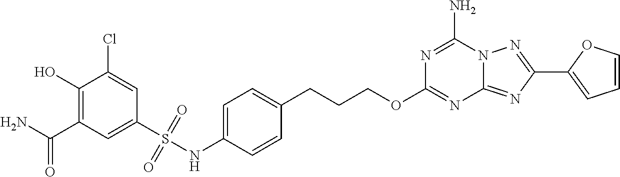 [SMILES: NC(=O)c1cc(S(=O)(=O)Nc2ccc(CCCOc3nc(N)n4nc(-c5ccco5)nc4n3)cc2)cc(Cl)c1O]

The title compound was prepared by a reaction of intermediate 25b (0.1 g) and 3-carbamoyl-4-hydroxybenzenesulfonyl chloride (4a, 0.11 g, 0.42 mmol) by following the general procedure described in in step (ii) of Example A1 as white solid (0.012 g, 7%). LCMS: m/z 584.1 (Mc); HPLC: 99.15%; ¹H NMR (400 MHz, DMSO-d₆): δ 15.05 (s, 1H), 10.94 (s, 1H), 9.04 (s, 1H), 8.89 (s, 1H), 8.69 (s, 1H), 8.41 (s, 1H), 6.87 (d, J=2.4 Hz, 1H), 7.91 (dd, J=0.8 Hz, 0.8 Hz, 1H), 7.86 (d, J=2 Hz, 1H), 7.14-7.12 (m, 3H), 7.03-7.01 (m, 2H), 6.71-6.70 (m, 1H), 4.26-4.23 (m, 2H), 2.64-2.60 (m, 2H), 1.98-1.91 (m, 2H) ppm.

### Example A13: 5-(N-(4-(3-((7-amino-2-(furan-2-yl)-[1,2,4]triazolo[1,5-a][1,3,5]triazin-5-yl)amino)propyl)-3-hydroxyphenyl)sulfamoyl)-3-chloro-2-hydroxybenzamide

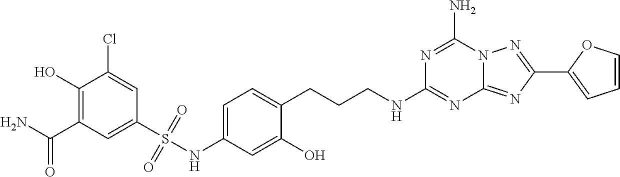 [SMILES: NC(=O)c1cc(S(=O)(=O)Nc2ccc(CCCNc3nc(N)n4nc(-c5ccco5)nc4n3)c(O)c2)cc(Cl)c1O]

### Step (i): Synthesis of (E)-3-(2-hydroxy-4-nitrophenyl)acrylonitrile (27)

To a stirred solution of LiCl.H₂O (0.9 g, 37.8 mmol) and LiOH (6.5 g, 108 mmol) in water; intermediate 5 (3 g, 17.9 mmol) 2-bromoacetonitrile (26, 3.8 g, 32 mmol) and PPh₃ (8.5 g, 32 mmol) were added. It was then heated at 100° C. for 0.5 h. Upon completion of the reaction (as indicated by TLC), the reaction mixture water was added and extracted with EtOAc for three times. The combined organic layer was washed with brine, dried over Na₂SO₄ and volatiles were removed under vacuum to provide the crude. It was then purified by combi-flash silica-gel chromatography using 0-10% EtOAc/hexane as eluent to obtain the title compound (2.4 g, 70%). LCMS: m/z 189.1 (M−H⁺); HPLC: 99.81%; ¹H NMR (400 MHz, DMSO-d₆): δ 11.58 (s, 1H), 7.81-7.79 (m, 1H), 7.73-7.69 (m, 3H), 6.68 (d, J=16.8 Hz, 1H) ppm.

### Step (ii): Synthesis of 3-(4-amino-2-hydroxyphenyl)propanenitrile (28)

The title compound was prepared by a reaction intermediate 27 (1.1 g) and 10% Palladium on charcoal (0.3 g) by following the general procedure described in step (iii) of Example A5 to obtain intermediate 28 (0.9 g, crude). LCMS: m/z 163.1 (M+H⁺).

### Step (iii): Synthesis of 5-amino-2-(3-aminopropyl)phenol (29)

To a stirred solution of intermediate 28 (0.9 g, 5.5 mmol) in EtOH (10 mL), Raney nickel (3 g) was added at RT. After stirring for 10 min hydrazine hydrate (1.6 g, 33 mmol) was added and the resulting mixture was stirred at 60° C. for 1 h. After 1 h, the reaction mixture was cooled to RT followed by addition of hydrazine hydrate (1.6 g, 33 mmol) and the reaction was further heated at 60° C. for 1 h. Upon completion of the reaction (as indicated by TLC), the reaction mixture was filtered through Celite® bed. The Celite® bed was washed with MeOH (25 ml×2) and combined organic layer was dried under vacuum to obtain the title compound (0.85 g, crude). LC-MS: m/z 167.2 (M+H⁺).

### Step (iv): Synthesis of 5-amino-2-(3-((7-amino-2-(furan-2-yl)-[1,2,4]triazolo[1,5-a][1,3,5]triazin-5-yl)amino)propyl)phenol (30)

The title compound was prepared by a reaction of 2-(furan-2-yl)-5-(methylsulfonyl)-[1,2,4]triazolo[1,5-a][1,3,5]triazin-7-amine (1, 0.35 g) and intermediate 29 (0.26 g, 1.62 mmol)) by following the general procedure described in step (i): Method B of preparation of Examples A1 to A4 (0.38 g, crude). LCMS: m/z 367.2 (M+H⁺).

### Step (v): Synthesis of 5-(N-(4-(3-((7-amino-2-(furan-2-yl)-[1,2,4]triazolo[1,5-a][1,3,5]triazin-5-yl)amino)propyl)-3-hydroxyphenyl)sulfamoyl)-3-chloro-2-hydroxybenzamide (Example A13)

The title compound was prepared by a reaction of intermediate 30 (0.38 g) and 3-carbamoyl-4-hydroxybenzenesulfonyl chloride (4a, 0.307 g, 1.14 mmol) by following the general procedure described in in step (ii) of Example A1 as white solid (0.055 g, 9%). LCMS: m/z 600.1 (M+H⁺); HPLC: 99.92%; ¹H NMR (400 MHz, DMSO-d₆): δ 15.12 (s, 1H), 9.90 (s, 1H), 9.39 (s, 1H), 9.01 (bs, 1H) 8.26-7.95 (m, 3H), 7.86-7.83 (m, 2H), 7.48-7.38 (m, 1H), 7.04-7.03 (m, 1H), 6.94-6.92 (m, 1H), 6.67-6.66 (m, 1H), 6.57-6.47 (m, 2H), 3.24-3.19 (m, 2H), 2.45-2.42 (m, 2H), 1.71-1.68 (m, 2H) ppm.

### Preparation of Example A14 & A15

### Step (i): Synthesis of (E)-3-(3-fluoro-4-nitrophenyl)acrylonitrile (33a)

To a stirred solution of 4-bromo-2-fluoro-1-nitrobenzene (31a, 0.5 g) in 1,4-dioxane (5 mL) in a sealed tube fitted with Teflon® screw-cap, was added acrylonitrile (32) and TBAB and stirred for 10 min at RT. Then NaHCO₃ was added and the reaction mixture was degassed with N₂ for 10 min. Pd(OAC)₂ was added to the reaction mixture and it was then heated at 100° C. for 16 h. Upon completion of the reaction (as indicated by TLC), the reaction mixture was quenched with water and extracted with EtOAc for three times. The combined organic layer was washed with brine, dried over Na₂SO₄ and volatiles were removed under vacuum to provide the crude. It was then purified by combi-flash silica-gel chromatography using 0-10% EtOAc/hexane as eluent to obtain the title compound. Yield: (0.250 g, 57%). LC-MS: m/z 193.0 (M+H⁺); ¹H NMR (400 MHz, DMSO-d₆): δ 8.24 (dd, J=8.4 Hz, 8 Hz, 1H), 7.95 (dd, J=12.4 Hz, 1.6 Hz, 1H), 7.77 (d, J=16.4 Hz, 1H), 7.70 (d, J=8.4 Hz, 1H), 6.78 (d, J=16.8 Hz, 1H), ppm.

### Step (i-a): Synthesis of (E)-3-(2-fluoro-4-nitrophenyl)acrylonitrile (33b)

The title compound was prepared by a reaction of 1-bromo-2-fluoro-4-nitrobenzene (31b, 2.5 g) and acrylonitrile (32, 2.41 g, 45.45 mmol) by following the general procedure described in step (i): (0.400 g, 18.34%). LC-MS: m/z 191.9 (M+H⁺); ¹H NMR (400 MHz, DMSO-d₆): δ 8.26-8.22 (m, 1H), 8.17-8.15 (m, 1H), 8.08-8.04 (m, 1H), 7.79 (d, J=16.8 Hz, 1H), 6.76 (d, J=16.4 Hz, 1H), ppm.

### Step (ii): Synthesis of 3-(4-amino-3-fluorophenyl)propanenitrile (34a)

The title compound was prepared by a reaction of (E)-3-(3-fluoro-4-nitrophenyl)acrylonitrile (33a, 0.25 g) and 10% Palladium on charcoal (0.16 g) by following the general procedure described in in step (iii) of Example A5 (0.15 g, crude). LC-MS: m/z 165.1 (M+H⁺); ¹H NMR (400 MHz, DMSO-d₆): δ 6.78 (d, J=12.8 Hz, 1H), 6.70-6.63 (m, 2H), 4.84 (s, 2H), 2.46-2.43 (m, 4H), ppm.

### Step (ii-a): Synthesis of 3-(4-amino-2-fluorophenyl)propanenitrile (34b)

The title compound was prepared by a reaction of (E)-3-(2-fluoro-4-nitrophenyl)acrylonitrile (33b, 0.1 g) and 10% Palladium on charcoal (0.1 g) by following the general procedure described in in step (iii) of Example A5; (0.08 g, crude). LC-MS: m/z 165.1 (M+H⁺). ¹H NMR (400 MHz, DMSO-d₆): δ 6.97-6.93 (m, 1H), 6.34-6.28 (m, 2H), 5.30 (s, 2H), 2.72-2.65 (m, 4H), ppm.

### Step (iii): Synthesis of 4-(3-aminopropyl)-2-fluoroaniline (35a)

The title compound was prepared by a reaction of 3-(4-amino-3-fluorophenyl)propanenitrile (34a, 0.15 g), hydrazine hydrate (0.3 mL×2) and wet Raney Nickel (0.3 g) by following the procedure described in step (iii) of Example A13. Yield: 0.12 g, crude. LC-MS: m/z 169 (M+H⁺).

### Step (iii-a): Synthesis of 4-(2-aminopropyl)-2-fluoroaniline (35b)

The title compound was prepared by a reaction of 3-(4-amino-2-fluorophenyl)propanenitrile (34b, 0.08 g), hydrazine hydrate (0.2 mL×2) and wet Raney Nickel (0.2 g) by following the procedure described in step (iii) of Example A3; Yield: 0.070 g, crude. LC-MS: m/z 169.1 (M+H⁺).

### Step (iv): Synthesis of N⁵-(3-(4-amino-3-fluorophenyl)propyl)-2-(furan-2-yl)-[1,2,4]triazolo[1,5-a][1,3,5]triazine-5,7-diamine (36a)

The title compound was prepared by a reaction of 2-(furan-2-yl)-5-(methylsulfonyl)-[1,2,4]triazolo[1,5-a][1,3,5]triazin-7-amine (1, 0.120 g) and intermediate 35a (0.093 g, 0.557 mmol) by following the general procedure described in step (i): Method B of preparation of Example A1 to A4 (0.170 g, crude). LCMS: m/z 369 (M+H⁺); ¹H NMR (400 MHz, DMSO-d₆): δ 7.95 (s, 2H), 7.86 (s, 1H), 7.52-7.46 (m, 1H), 7.04-7.03 (m, 1H), 6.86-6.80 (m, 1H), 6.73-6.64 (m, 4H), 4.87 (s, 2H), 3.24-3.21 (m, 2H), 2.47-2.46 (m, 2H), 1.76-1.73 (m, 2H) ppm.

### Step (iv-a): Synthesis of N⁵-(3-(4-amino-2-fluorophenyl)propyl)-2-(furan-2-yl)-[1,2,4]triazolo[1,5-a][1,3,5]triazine-5,7-diamine (36b)

The title compound was prepared by a reaction of 2-(furan-2-yl)-5-(methylsulfonyl)-[1,2,4]triazolo[1,5-a][1,3,5]triazin-7-amine (1, 0.09 g) and intermediate 35b (0.07 g, 0.417 mmol)) by following the general procedure described in step (i): Method B of preparation of Examples A1 to A4. (0.120 g, crude). LCMS: m/z 369.1 (M+H⁺); ¹H NMR (400 MHz, DMSO-d₆): δ 7.95 (s, 2H), 7.85 (s, 1H), 7.52-7.46 (m, 1H), 7.04 (s, 1H), 6.90-6.88 (m, 1H), 6.66 (s, 1H), 6.31-6.26 (m, 2H), 5.14 (s, 2H), 3.27-3.24 (m, 2H), 2.46-2.44 (m, 2H), 1.74-1.70 (m, 2H) ppm.

### Example A14: 5-(N-(4-(3-((7-amino-2-(furan-2-yl)-[1,2,4]triazolo[1,5-a][1,3,5]triazin-5-yl)amino)propyl)-3-fluorophenyl)sulfamoyl)-3-chloro-2-hydroxybenzamide

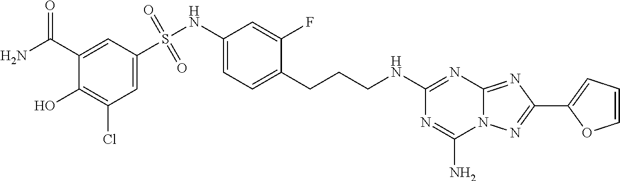 [SMILES: NC(=O)c1cc(S(=O)(=O)Nc2ccc(CCCNc3nc(N)n4nc(-c5ccco5)nc4n3)c(F)c2)cc(Cl)c1O]

The title compound was prepared by a reaction of intermediate 36a (170 mg) and 3-carbamoyl-4-hydroxybenzenesulfonyl chloride (4a, 149 mg, 0.55 mmol) by following the general procedure described in in step (ii) of Example A1 as white solid (0.040 g, 14.44%). LCMS: m/z 602.1 (M+H⁺); HPLC: 95.44%; ¹H NMR (400 MHz, DMSO-d₆): δ 15.12 (s, 1H), 9.95 (s, 1H), 9.05 (s, 1H), 8.39-8.21 (m, 4H), 7.88-7.86 (m, 2H), 7.50-7.47 (m, 1H), 7.11-7.01 (m, 4H), 6.68-6.66 (m, 1H), 3.26-3.21 (m, 2H), 2.54-2.53 (m, 2H), 1.79-1.76 (m, 2H) ppm.

### Example A15: 5-(N-(4-(3-((7-amino-2-(furan-2-yl)-[1,2,4]triazolo[1,5-a][1,3,5]triazin-5-yl)amino)propyl)-2-fluorophenyl)sulfamoyl)-3-chloro-2-hydroxybenzamide

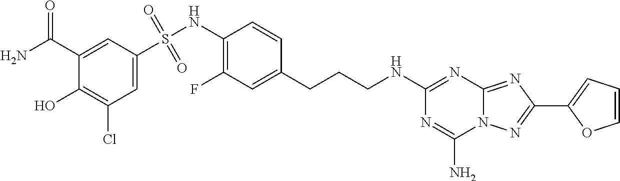 [SMILES: NC(=O)c1cc(S(=O)(=O)Nc2ccc(CCCNc3nc(N)n4nc(-c5ccco5)nc4n3)cc2F)cc(Cl)c1O]

The title compound was prepared by a reaction of intermediate 36b (120 mg) and 3-carbamoyl-4-hydroxybenzenesulfonyl chloride (4a, 0.105 g, 0.391 mmol) by following the general procedure described in step (ii) of Example A1 as white solid (0.003 g, 1.53%). LC-MS: m/z 602.1 (M+H⁺); HPLC: 99.86%; ¹H NMR (400 MHz, DMSO-d₆): δ 10.45 (s, 1H), 8.32 (s, 1H), 8.15 (s, 1H), 7.86 (s, 2H), 7.50-7.47 (m, 1H), 7.22-7.19 (m, 1H), 7.06-7.04 (m, 1H), 6.90-6.83 (m, 2H), 6.67 (s, 1H), 3.27-3.23 (m, 2H), 2.54-2.53 (m, 2H), 1.75-1.73 (m, 2H) ppm.

### Example A16: 5-(N-(4-(3-((7-amino-2-(furan-2-yl)-[1,2,4]triazolo[1,5-a][1,3,5]triazin-5-yl)amino)propyl)phenyl)sulfamoyl)-3-chloro-2-methoxybenzamide

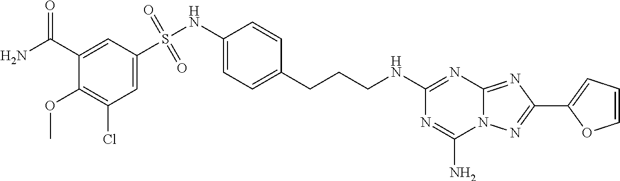 [SMILES: COc1c(Cl)cc(S(=O)(=O)Nc2ccc(CCCNc3nc(N)n4nc(-c5ccco5)nc4n3)cc2)cc1C(N)=O]

### Step (i): Synthesis of 3-chloro-2-methoxybenzamide (38)

To a stirred solution of 2-chloro-3-hydroxybenzamide (37, 0.5 g) in DMF (3 mL) at 0° C. was added NaH (0.1 g, 4.38 mmol) and the mixture was stirred for 10 min. Then MeI (0.41 g, 2.92) was and the resulting solution was stirred at RT for 2 h. Upon completion of the reaction (as indicated by TLC), the reaction mixture was quenched with water and extracted with EtOAc for three times. The combined organic layer was washed with brine, dried over Na₂SO₄ and volatiles were removed under vacuum to provide the crude. It was then purified by combi-flash silica-gel chromatography using 20-30% EtOAc/hexane as eluent to obtain the title compound (0.190 g, 37%). LC-MS: m/z 186.1 (M+H⁺).

### Step (ii): Synthesis of 3-carbamoyl-5-chloro-4-methoxybenzenesulfonyl chloride (39)

A solution of intermediate 38 (0.19 g) in chlorosulphonic acid (1 mL) was heated at 70° C. for 1 h. Upon completion of the reaction, the reaction mixture was poured into ice. The obtained solid precipitate was filtered and dried to obtain the title compound (0.28 g, 96%). It was used without any further purification for the next step. LCMS: m/z 284.0 (M⁺); ¹H NMR (400 MHz, DMSO-d₆): δ 7.86 (bs, 1H), 7.66 (d, J=2 Hz, 1H), 7.64 (d, J=2 Hz, 1H), 7.62 (bs, 1H), 3.81 (s, 3H) ppm.

### Step (iii): Synthesis of 5-(N-(4-(3-((7-amino-2-(furan-2-yl)-[1,2,4]triazolo[1,5-a][1,3,5]triazin-5-yl)amino)propyl)phenyl)sulfamoyl)-3-chloro-2-methoxybenzamide (Example A16)

The title compound was prepared by a reaction of intermediate 25a (0.3 g) and 3-carbamoyl-5-chloro-4-methoxybenzenesulfonyl chloride (39, 0.265 g, 0.94 mmol) by following the general procedure described in step (ii) Example A1 as white solid (0.035 g, 7%). LCMS: m/z 586.1 (M+H⁺); HPLC: 99.07%; ¹H NMR (400 MHz, DMSO-d₆): δ 10.28 (bs, 1H), 8.17-8.08 (m, 2H), 7.94 (s, 1H), 7.85-7.80 (m, 4H), 7.53-7.485 (m, 1H), 7.13-7.11 (m, 2H), 7.04-6.99 (m, 3H), 6.66 (s, 1H), 3.85 (s, 3H), 3.24-3.22 (m, 2H), 2.52-2.50 (m, 2H), 1.77-1.74 (m, 2H) ppm.

### Example A17: 5-(N-(4-(3-((7-amino-2-(furan-2-yl)-[1,2,4]triazolo[1,5-a][1,3,5]triazin-5-yl)(methyl)amino)propyl)phenyl)sulfamoyl)-3-chloro-2-hydroxybenzamide

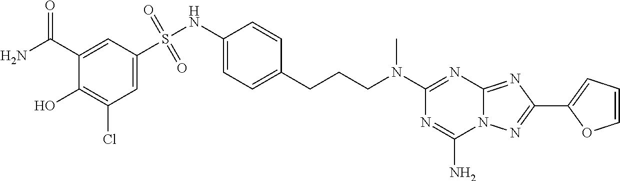 [SMILES: CN(CCCc1ccc(NS(=O)(=O)c2cc(Cl)c(O)c(C(N)=O)c2)cc1)c1nc(N)n2nc(-c3ccco3)nc2n1]

### Step (i): Synthesis of tert-butyl (3-(4-nitrophenyl)propyl)carbamate (41)

To a stirred solution of 3-(4-nitrophenyl)propan-1-amine (40, 1.0 g) in DCM (10 mL) was added triethyl amine (1 mL, 8.333 mmol) and di-tert-butyl dicarbonate (1.5 ml, 6.666 mmol). The reaction mixture was stirred at RT for 16 h. Upon completion of the reaction (as indicated by TLC), the reaction mixture was quenched with water and extracted with DCM for three times. The combined organic layer was washed with brine, dried over Na₂SO₄ and volatiles were removed under vacuum to provide the crude. It was then purified by combi-flash silica-gel chromatography using 0-20% EtOAc/hexane as eluent to obtain the title compound (0.7 g, 46%). ¹H NMR (400 MHz, DMSO-d₆): 8.24 (d, J=8.4 Hz, 2H), 7.50 (d, J=8.4 Hz, 2H), 6.90 (bs, 1H), 2.96-2.91 (m, 2H), 2.72-2.68 (m, 2H), 1.74-1.69 (m, 2H), 1.37 (s, 9H) ppm.

### Step (ii): Synthesis of tert-butyl methyl(3-(4-nitrophenyl)propyl)carbamate (42)

To a stirred solution of tert-butyl (3-(4-nitrophenyl)propyl)carbamate (41, 0.7 g) in DMF (3 mL) at 0° C. was added NaH (0.054 g, 3.75 mmol) and the mixture was stirred for 10 min. Then MeI (0.24 ml, 3.75 mmol) was and the resulting solution was stirred at RT for 2 h. Upon completion of the reaction (as indicated by TLC), the reaction mixture was quenched with water and extracted with EtOAc for three times. The combined organic layer was washed with brine, dried over Na₂SO₄ and volatiles were removed under vacuum to provide the crude. It was then purified by combi-flash silica-gel chromatography using 0-10% EtOAc/hexane as eluent to obtain the title compound (0.17 g, 23%). ¹H NMR (400 MHz, DMSO-d₆): 8.15 (d, J=8.8 Hz, 2H), 7.51 (d, J=8.8 Hz, 2H), 3.19-3.16 (m, 2H), 2.76 (s, 3H), 2.70-2.66 (m, 2H), 1.81-1.71 (m, 2H), 1.37-1.35 (m, 9H) ppm.

### Step (iii): Synthesis of N-methyl-3-(4-nitrophenyl) propan-1-amine (43)

To a stirred solution of tert-butyl methyl (3-(4-nitrophenyl) propyl) carbamate (42, 0.17 g) in 1,4 dioxane (4 mL) was added 4M HCl in dioxane (2 mL) at RT. The resulting reaction mixture was stirred at RT for 1 h. Upon completion of the reaction (as indicated by TLC), the reaction mixture was concentrated under vacuum and washed with ether and pentane to provide the crude. It was used in next step without any further purification (0.120 g, crude). LC-MS: m/z 195.2 (M+H⁺).

### Step (iv): Synthesis of 2-(furan-2-yl)-N⁵-methyl-N⁵-(3-(4-nitrophenyl)propyl)-[1,2,4]triazolo[1,5-a][1,3,5]triazine-5,7-diamine (44)

The title compound was prepared by a reaction of 2-(furan-2-yl)-5-(methylsulfonyl)-[1,2,4]triazolo[1,5-a][1,3,5]triazin-7-amine (1, 0.120 g) and intermediate 43 (0.093 g, 0.557 mmol) by following the general procedure described in step (i): Method B of preparation of Examples A1 to A4; (0.170 g, 85%). LCMS: m/z 395.1 (M+H⁺).

### Step (v): Synthesis of N⁵-(3-(4-aminophenyl)propyl)-2-(furan-2-yl)-N⁵-methyl-[1,2,4]triazolo[1,5-a][1,3,5]triazine-5,7-diamine (45)

To a stirred solution of 10% palladium on charcoal (0.05 g) in MeOH:THF (1.5:1.5 mL), a solution of intermediate (44) (0.22 g) in MeOH:THF (1.5:1.5 mL) was added. The resulting suspension was stirred at RT for 5 h under H₂ balloon (50 psi). Upon completion of the reaction (as indicated by TLC), the reaction mixture was filtered through Celite® bed. The Celite® bed was washed with MeOH:THF (15 ml×2) and combined organic layer was dried under vacuum to obtain the title compound (0.1 g, crude). It was used in next step without any further purification. (0.1 g, crude); LC-MS: m/z 365.2 (M+H⁺). ¹H NMR (400 MHz, DMSO-d₆): δ 7.86 (s, 1H), 7.04 (d, J=3.2 Hz, 1H), 6.87 (d, J=8.0 Hz, 2H), 6.67-6.65 (m, 1H), 6.48 (d, J=8.4 Hz, 2H), 4.80 (s, 2H), 3.59 (bs, 2H), 3.10-3.07 (m, 3H), 2.48-2.42 (m, 2H), 1.81-1.77 (m, 2H) ppm.

### Step (vi): 5-(N-(4-(3-((7-amino-2-(furan-2-yl)-[1,2,4]triazolo[1,5-a][1,3,5]triazin-5-yl)(methyl)amino)propyl)phenyl)sulfamoyl)-3-chloro-2-hydroxybenzamide (Example A17)

The title compound was prepared by a reaction of intermediate 45 (0.1 g) and 3-carbamoyl-4-hydroxybenzenesulfonyl chloride (4a, 0.096 g, 0.357 mmol) by following the general procedure described in step (ii) of Example A1 as white solid (0.042 g, 26%). LC-MS: m/z 598.2 (M); HPLC: 96.78%; ¹H NMR (400 MHz, DMSO-d₆): δ 15.02 (s, 1H), 10.02 (s, 1H), 9.05 (bs, 1H), 8.20-8.35 (m, 3H), 7.86 (s, 1H), 7.12-7.00 (m, 4H), 6.67 (s, 1H), 3.57 (bs, 2H), 3.07-3.04 (m, 3H), 2.52-2.50 (m, 2H), 1.77-1.83 (m, 2H) ppm.

### Example B4: N-(4-(3-((7-amino-2-(furan-2-yl)-[1,2,4]triazolo[1,5-a][1,3,5]triazin-5-yl)amino)propyl)phenyl)-8-chloro-2,4-dioxo-3,4-dihydro-2H-benzo[e][1,3]oxazine-6-sulfonamide

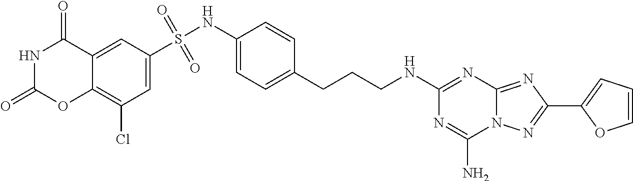 [SMILES: Nc1nc(NCCCc2ccc(NS(=O)(=O)c3cc(Cl)c4oc(=O)[nH]c(=O)c4c3)cc2)nc2nc(-c3ccco3)nn12]

### Step (i): Synthesis of 8-chloro-2,4-dioxo-3,4-dihydro-2H-benzo[e][1,3]oxazine-6-sulfonyl chloride (46)

To a stirred solution of intermediate 4a (1.5 g, 5.56 mmol) in toluene (10 mL), oxalyl chloride (0.78 g, 6.13 mmol) was added at 0° C. The resulting mixture was heated at 100° C. for 5 h. Upon completion of the reaction (as indicated by TLC), the reaction mixture was quenched with ice-water and the solid precipitate was filtered and dried under vacuum to provide the crude (1.5 g, crude). It was used for the next step without any further purification. LCMS: m/z 294.8 (M−H⁺). Step

### Step (ii): Synthesis of N-(4-(3-((7-amino-2-(furan-2-yl)-[1,2,4]triazolo[1,5-a][1,3,5]triazin-5-yl)amino)propyl)phenyl)-8-chloro-2,4-dioxo-3,4-dihydro-2H-benzo[e][1,3]oxazine-6-sulfonamide (Example B4)

The title compound was prepared by a reaction of intermediate 25a (0.28 mg) and intermediate 46 (0.35 g, 1.19 mmol) by following the general procedure described in step (ii) of Example A1 as white solid (0.015 g, 2%). LC-MS: m/z 610.1 (M+); HPLC: 98.01%; ¹H NMR (400 MHz, DMSO-d₆): δ 14.59 (s, 1H), 9.70 (s, 1H), 8.12 (bs, 1H), 8.03 (d, J=2.8 Hz, 1H), 7.85 (s, 1H), 7.47-7.43 (m, 2H), 7.08-6.96 (m, 5H), 6.67-6.66 (m, 1H), 6.54 (s, 1H), 3.25-3.21 (m, 2H), 2.54-2.52 (m, 2H), 1.78-1.75 (m, 2H) ppm.

### Example C1: 5-(N-(4-((2-((7-amino-2-(furan-2-yl)-[1,2,4]triazolo[1,5-a][1,3,5]triazin-5-yl)amino)ethyl)amino)phenyl)sulfamoyl)-3-chloro-2-hydroxybenzamide

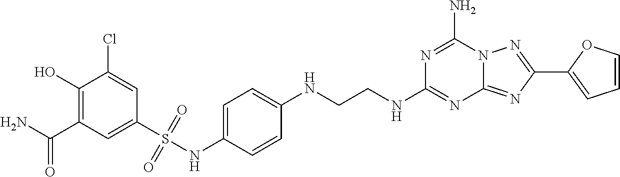 [SMILES: NC(=O)c1cc(S(=O)(=O)Nc2ccc(NCCNc3nc(N)n4nc(-c5ccco5)nc4n3)cc2)cc(Cl)c1O]

### Step (i): Synthesis of N⁵-(2-((4-aminophenyl)amino)ethyl)-2-(furan-2-yl)-[1,2,4]triazolo[1,5-a][1,3,5]triazine-5,7-diamine (47)

The title compound was prepared by a reaction of 2-(furan-2-yl)-5-(methylsulfonyl)-[1,2,4]triazolo[1,5-a][1,3,5]triazin-7-amine (1, 0.3 g) and intermediate 46 (0.19 g, 1.07 mmol) by following the general procedure described in step (i): Method B of preparation of Examples A1 to A4 (0.18 g, 48%). LCMS: m/z 352.2 (M+H⁺); ¹H NMR (400 MHz, DMSO-d₆): δ 8.13 (bs, 2H), 7.90-7.86 (m, 2H), 7.42-7.38 (m, 1H), 7.04-6.89 (m, 3H), 6.67 (s, 1H), 5.14 (s, 2H), 3.53-3.50 (m, 2H), 2.81-2.78 (m, 2H) ppm.

### Step (ii): Synthesis of 5-(N-(4-((2-((7-amino-2-(furan-2-yl)-[1,2,4]triazolo[1,5-a][1,3,5]triazin-5-yl)amino)ethyl)amino)phenyl)sulfamoyl)-3-chloro-2-hydroxybenzamide (Example C1)

The title compound was prepared by a reaction of intermediate 47 (0.08 g) and 3-carbamoyl-4-hydroxybenzenesulfonyl chloride (4a, 0.06 g, 0.244 mmol) by following the general procedure described in step (ii) of Example A1 as white solid (0.010 g, 8%). LCMS: m/z 585.1 (M⁺); HPLC: 99.90%; ¹H NMR (400 MHz, DMSO-d₆): δ 15.01 (s, 1H), 12.78 (s, 1H), 10.72 (s, 1H), 8.95 (s, 1H), 8.13-7.86 (m, 4H), 7.48-7.39 (m, 1H), 7.05 (d, J=2.8 Hz, 1H), 6.76 (d, J=8.8 Hz, 2H), 6.67-6.44 (m, 3H), 5.60 (s, 2H), 3.39-3.37 (m, 2H), 3.13-3.11 (m, 2H) ppm.

### Example C2: 5-(N-(3-((2-((7-amino-2-(furan-2-yl)-[1,2,4]triazolo[1,5-a][1,3,5]triazin-5-yl)amino)ethyl)amino)phenyl)sulfamoyl)-3-chloro-2-hydroxybenzamide

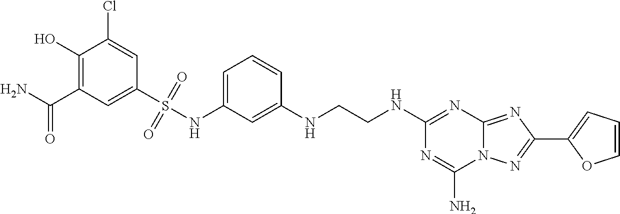 [SMILES: NC(=O)c1cc(S(=O)(=O)Nc2cccc(NCCNc3nc(N)n4nc(-c5ccco5)nc4n3)c2)cc(Cl)c1O]

### Step (i): Synthesis of N′-(2-aminoethyl)benzene-1,3-diamine (49)

The title compound was prepared by a reaction N¹-(3-nitrophenyl)ethane-1,2-diamine (48, 2.5 g) and 10% Palladium on charcoal (0.5 g) by following the general procedure described in step (iii) of Example A5 to obtain the title compound (1.5 g, 71%). LC-MS: m/z 152.2 (M+H⁺); ¹H NMR (400 MHz, DMSO-d₆): δ 6.71-6.67 (m, 1H), 5.82-5.77 (m, 3H), 5.16-5.13 (m, 1H), 4.68 (m, 2H), 3.16 (s, 1H), 2.92-2.88 (m, 2H), 2.68-2.65 (m, 2H) ppm.

### Step (ii): Synthesis of N⁵-(2-((3-aminophenyl)amino)ethyl)-2-(furan-2-yl)-[1,2,4]triazolo[1,5-a][1,3,5]triazine-5,7-diamine (50)

The title compound was prepared by a reaction of 2-(furan-2-yl)-5-(methylsulfonyl)-[1,2,4]triazolo[1,5-a][1,3,5]triazin-7-amine (1, 0.3 g) and intermediate 49 (0.19 g, 1.28 mmol) by following the general procedure described in step (i): Method B of preparation of Examples A1 to A4 (0.18 g, 48%). LCMS: m/z 352.2 (M+H⁺); ¹H NMR (400 MHz, DMSO-d₆): δ 7.87 (bs, 2H), 7.47-7.46 (m, 2H), 7.05-7.04 (m, 1H), 6.73-6.67 (m, 2H), 4.10 (s, 2H), 3.43-3.40 (m, 2H), 3.17-3.16 (m, 2H) ppm.

### Step (iii): 5-(N-(3-((2-((7-amino-2-(furan-2-yl)-[1,2,4]triazolo[1,5-a][1,3,5]triazin-5-yl)amino)ethyl)amino)phenyl)sulfamoyl)-3-chloro-2-hydroxybenzamide (Example C2)

The title compound was prepared by a reaction of intermediate 50 (0.06 g) and 3-carbamoyl-4-hydroxybenzenesulfonyl chloride (4a, 0.05 g, 0.188 mmol) by following the general procedure described in step (ii) of Example A1 as off-white solid (0.020 g, 20%). LCMS: m/z 585.1 (M⁺); HPLC: 99.73%; ¹H NMR (400 MHz, DMSO-d₆): δ 15.00 (s, 1H), 9.83 (s, 1H), 9.04 (s, 1H), 8.30-8.13 (m, 4H), 7.86 (bs, 2H), 7.46-7.40 (m, 1H), 7.07 (d, J=2.8 Hz, 1H), 6.92 (dd, J=8 Hz, 8 Hz, 1H), 6.67 (s, 1H), 6.50-6.29 (m, 3H), 5.75 (bs, 1H), 3.40-3.38 (m, 2H), 3.13-3.11 (m, 2H) ppm.

### Example D1: 5-(N-(1-(2-((7-amino-2-(furan-2-yl)-[1,2,4]triazolo[1,5-a][1,3,5]triazin-5-yl)amino)ethyl)-1H-indol-6-yl)sulfamoyl)-3-chloro-2-hydroxybenzamide

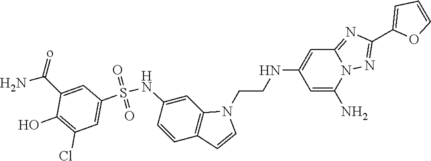

### Step (i): Synthesis of N⁵-(2-(6-amino-1H-indol-1-yl)ethyl)-2-(furan-2-yl)-[1,2,4]triazolo[1,5-a][1,3,5]triazine-5,7-diamine (52)

The title compound was prepared by a reaction of 2-(furan-2-yl)-5-(methylsulfonyl)-[1,2,4]triazolo[1,5-a][1,3,5]triazin-7-amine (1, 0.15 g) and 1-(2-aminoethyl)-1H-indol-6-amine (51, 0.112 g, 0.642 mmol) by following the general procedure described in step (i): Method B of preparation of Examples A1 to A4 (0.15 g, 75%). LCMS: m/z 375.1 (M+H⁺).

### Step (ii): Synthesis of 5-(N-(1-(2-((7-amino-2-(furan-2-yl)-[1,2,4]triazolo[1,5-a][1,3,5]triazin-5-yl)amino)ethyl)-1H-indol-6-yl)sulfamoyl)-3-chloro-2-hydroxybenzamide (Example D1)

The title compound was prepared by a reaction of intermediate 52 (0.07 g) and 3-carbamoyl-5-chloro-4-hydroxybenzenesulfonyl chloride (4a, 0.106, 0.28 mmol) by following the general procedure described in step (ii) of Example A1 as white solid (0.010 g, 6%). LCMS: m/z 609.0 (M+H⁺); HPLC: 99.81%; ¹H NMR (400 MHz, DMSO-d₆): δ 15.08 (bs, 1H), 9.99 (s, 1H), 9.90 (s, 1H), 9.37 (s, 1H), 9.20 (s, 1H), 8.47-8.41 (m, 2H), 8.30-8.13 (m, 1H), 7.90-7.87 (m, 1H), 7.47-7.38 (m, 1H), 7.38-7.36 (m, 1H), 7.28-7.07 (m, 3H), 6.68-6.66 (m, 2H), 6.37-6.33 (m, 1H), 4.33-4.30 (m, 2H), 3.59-3.55 (m, 2H) ppm.

### Example D2: 5-(N-(1-(2-((7-amino-2-(furan-2-yl)-[1,2,4]triazolo[1,5-a][1,3,5]triazin-5-yl)amino)ethyl)-1H-indazol-4-yl)sulfamoyl)-3-chloro-2-hydroxybenzamide

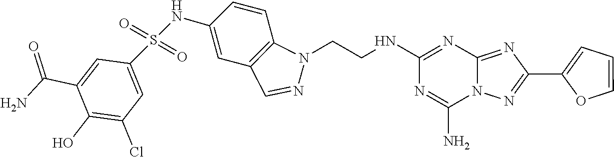 [SMILES: NC(=O)c1cc(S(=O)(=O)Nc2ccc3c(cnn3CCNc3nc(N)n4nc(-c5ccco5)nc4n3)c2)cc(Cl)c1O]

### Step (i): Synthesis of tert-butyl (2-(4-nitro-1H-indazol-1-yl)ethyl)carbamate (55)

To a solution of 4-nitro-1H-indazole (54, 1 g, 6.13 mmol) in DMF (5 mL) at RT, Cs₂CO₃ (4 g, 12.26 mmol) and KI (0.1 g, 0.61 mmol) were added the mixture was stirred at RT for 10 min. Then tert-butyl (2-bromoethyl) carbamate ((53, 1.77 g, 7.96 mmol) was added and the resulting mixture was heated at 80° C. for 3 h. Upon completion of the reaction as indicated by TLC, the mixture was quenched with water and extracted with EtOAc (3×100 mL). The combined organic extracts were washed with brine and dried over anhydrous sodium sulfate. The crude was purified with silica-gel chromatography using 15% EtOAc/hexane as eluent to obtain the title compound as (1.1 g, 61%). LC-MS: m/z 307.1 (M+H⁺); ¹H NMR (400 MHz, d₆-DMSO): δ 8.84 (s, 1H), 8.58 (d, J=8.4 Hz, 1H), 8.34 (d, J=8 Hz, 1H), 7.87 (dd, J=8.4 Hz, 8 Hz, 1H), 1.67 (s, 9H) ppm.

### Step (ii): Synthesis of tert-butyl (2-(4-amino-1H-indazol-1-yl)ethyl)carbamate (56)

The title compound was prepared by a reaction intermediate 55 (1.1 g) and 10% Palladium on charcoal (0.5 g) by following the general procedure described in step (iii) of Example A5 to obtain the intermediate 56 (0.8 g, 80%). LC-MS: m/z 277.2 (M+H⁺).

### Step (iii): Synthesis of 1-(2-aminoethyl)-1H-indazol-4-amine (57)

To a stirred solution of intermediate 56 (0.8 g) in DCM (10 mL) was added 4M HCl in 1,4-dioxane (8 mL) at 0° C. under nitrogen atmosphere. The resulting mixture was stirred at RT for 2 h. Upon completion of the reaction (as indicated by TLC) volatiles were removed under vacuum and the crude solid obtained was washed with diethyl ether and pentane four times to obtain the title compound (0.7, crude). It was used in next step without any further purification LCMS: m/z 177.2 (M+H⁺).

### Step (iv): Synthesis of N⁵-(2-(4-amino-1H-indazol-1-yl)ethyl)-2-(furan-2-yl)-[1,2,4]triazolo[1,5-a][1,3,5]triazine-5,7-diamine (58)

The title compound was prepared by a reaction of 2-(furan-2-yl)-5-(methylsulfonyl)-[1,2,4]triazolo[1,5-a][1,3,5]triazin-7-amine (1, 0.15 g) and intermediate 57 (0.17 g, 0.99 mmol) by following the general procedure described in step (i): Method B of preparation of Examples A1 to A4; (0.14 g, 70%). LCMS: m/z 377.0 (M+H⁺); ¹H NMR (400 MHz, DMSO-d₆): δ 8.09 (s, 1H), 7.95-7.88 (m, 2H), 7.50-7.41 (m, 1H), 7.08-6.98 (m, 1H), 6.69-6.59 (m, 3H), 6.14-6.12 (m, 1H), 5.77 (s, 2H), 5.69 (s, 1H), 4.47-4.44 (m, 2H), 3.67-3.61 (m, 2H) ppm.

### Step (iv): Synthesis of 5-(N-(1-(2-((7-amino-2-(furan-2-yl)-[1,2,4]triazolo[1,5-a][1,3,5]triazin-5-yl)amino)ethyl)-1H-indazol-4-yl)sulfamoyl)-3-chloro-2-hydroxybenzamide (Example D2)

The title compound was prepared by a reaction of intermediate 58 (0.1 g) and 3-carbamoyl-4-hydroxybenzenesulfonyl chloride (4a, 0.078 g, 0.188 mmol) by following the general procedure described in step (ii) of Example A1 as white solid (0.020 g, 16%). LCMS: m/z 610.1 (M+); HPLC: 99.32%; ¹H NMR (400 MHz, DMSO-d₆): δ 10.50 (s, 1H), 8.32 (s, 1H), 8.21 (s, 1H), 7.88 (s, 1H), 7.52-7.41 (m, 1H), 7.25-7.20 (m, 2H), 7.07-7.06 (m, 1H), 6.93-6.91 (m, 1H), 6.69-6.68 (m, 1H), 4.55-4.54 (m, 2H), 3.63-3.61 (m, 2H) ppm.

### Example A18: 5-(N-(4-(4-((7-amino-2-(furan-2-yl)-[1,2,4]triazolo[1,5-a][1,3,5]triazin-5-yl)amino)butyl)phenyl)sulfamoyl)-3-chloro-2-hydroxybenzamide

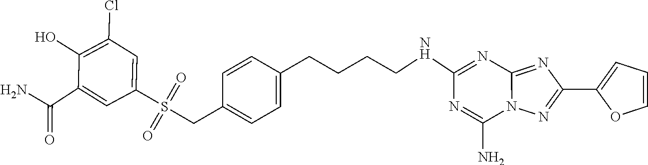 [SMILES: NC(=O)c1cc(S(=O)(=O)Cc2ccc(CCCCNc3nc(N)n4nc(-c5ccco5)nc4n3)cc2)cc(Cl)c1O]

### Step (i): Synthesis 4-(4-aminobutyl)aniline (60)

The title compound was prepared by a reaction 4-(4-nitrophenyl)butan-1-amine (59, 0.35 g) and 10% Palladium on charcoal (0.1 g) by following the general procedure described in step (iii) of Example A5: (0.119 g, crude). LC-MS: m/z 165.2 (M+H⁺).

### Step (ii): Synthesis of N⁵-(4-(4-aminophenyl)butyl)-2-(furan-2-yl)-[1,2,4]triazolo[1,5-a][1,3,5]triazine-5,7-diamine (61)

The title compound was prepared by a reaction of 2-(furan-2-yl)-5-(methylsulfonyl)-[1,2,4]triazolo[1,5-a][1,3,5]triazin-7-amine (1, 0.15 g) and intermediate 60 (0.119 g, 0.727 mmol) by following the general procedure described in step (i): Method B of preparation of Examples A1 to A4. Yield: 0.1 g, 51%. LCMS: m/z 365.2 (M+H⁺).

### Step (iii): Synthesis of 5-(N-(4-(4-((7-amino-2-(furan-2-yl)-[1,2,4]triazolo[1,5-a][1,3,5]triazin-5-yl)amino)butyl)phenyl)sulfamoyl)-3-chloro-2-hydroxybenzamide (Example A18)

The title compound was prepared by a reaction of intermediate 61 (0.1 g) and 3-carbamoyl-4-hydroxybenzenesulfonyl chloride (4a, 0.095 g, 0.357 mmol) by following the general procedure described in step (ii) of Example A1 as white solid (0.007 g, 4%). LCMS: m/z 598.10 (M+H⁺); HPLC: 98.38%; ¹H NMR (400 MHz, DMSO-d₆): δ 15.13 (s, 1H), 9.52 (s, 1H), 9.10 (s, 1H), 8.36-8.18 (m, 4H), 7.90-7.86 (m, 2H), 7.50-7.41 (m, 2H), 7.24-7.03 (m, 2H), 6.83-6.81 (m, 1H), 6.67-6.66 (m, 1H), 3.26-3.24 (m, 2H), 2.57-2.52 (m, 2H), 1.50-1.44 (m, 4H) ppm.

### Example A19: 5-(N-(4-(3-((5-amino-2-(furan-2-yl)-[1,2,4]triazolo[1,5-c]pyrimidin-7-yl)amino)propyl)phenyl)sulfamoyl)-3-chloro-2-hydroxybenzamide

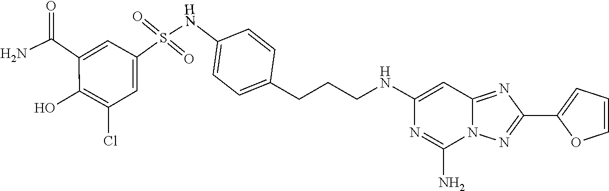 [SMILES: NC(=O)c1cc(S(=O)(=O)Nc2ccc(CCCNc3cc4nc(-c5ccco5)nn4c(N)n3)cc2)cc(Cl)c1O]

The compound, 7-chloro-2-(furan-2-yl)-[1,2,4]triazolo[1,5-c]pyrimidin-5-amine (intermediate 62) was prepared by following the procedure described in PCT publication WO2003048163.

### Step (i): Synthesis of N⁷-(3-(4-aminophenyl)propyl)-2-(furan-2-yl)-[1,2,4]triazolo[1,5-c]pyrimidine-5,7-diamine (63)

A solution of intermediate 62 (0.35 g, 1.48 mmol) in DMSO (2 mL) was taken in a sealed-tube fitted with Teflon® coated screw cap and intermediate 24a (0.26 g, 1.62 mmol) and CsF (0.46 g, 2.96 mmol) was added to it. The resulting solution was sealed and stirred at 110° C. for 2 h. Upon completion of the reaction (as indicated by TLC), the reaction mixture was quenched with ice-water and the solid precipitate was filtered to provide the crude (0.1 g, crude). LCMS: m/z 351.1 (M+H⁺).

### Step (i): Synthesis of 5-(N-(4-(3-((5-amino-2-(furan-2-yl)-[1,2,4]triazolo[1,5-c]pyrimidin-7-yl)amino)propyl)phenyl)sulfamoyl)-3-chloro-2-hydroxybenzamide (Example A19)

The title compound was prepared by a reaction of intermediate 63 (0.1 g) and 3-carbamoyl-4-hydroxybenzenesulfonyl chloride (4a, 0.13 g, 0.37 mmol) by following the general procedure described in step (ii) of Example A1 as white solid (0.003 g, 2%). LCMS: m/z 584.1 (M+H⁺); HPLC: 91.99%; ¹H NMR (400 MHz, DMSO-d₆): δ 10.58 (d, J=5.6 Hz, 1H), 9.60 (s, 1H), 8.42 (bs, 2H), 8.95 (s, 1H), 7.84 (s, 1H), 7.45-7.40 (m, 2H), 7.06-6.96 (m, 7H), 5.63 (s, 1H), 3.16-3.13 (m, 2H), 2.00-1.98 (m, 2H), 1.77-1.73 (m, 2H) ppm.

### Preparation of Example A20-A23

The compound, 5,7-dichloro-2-(furan-2-yl)-[1,2,4]triazolo[1,5-a]pyrimidine (intermediate 64) was prepared by following the procedure described in European Journal of Medicinal Chemistry, 92 (2015); 754-765.

### Step (i): Synthesis of 5-chloro-2-(furan-2-yl)-[1,2,4]triazolo[1,5-a]pyrimidin-7-amine (65)

A solution of intermediate 64 (1.3 g) in 7M NH₃ in MeOH (20 mL) was taken in a steel bomb. The resulting solution was sealed and stirred at 50° C. for 3 h. Upon completion of the reaction (as indicated by TLC), the volatiles were removed under vacuum to provide the crude intermediate 65 (0.89 g, crude). It was used in next step without any further purification. LC-MS: m/z 236.0 (M+H⁺). ¹H NMR (400 MHz, DMSO-d₆): δ 8.45 (bs, 2H), 7.93 (d, J=1.2 Hz, 1H), 7.18 (d, J=3.6 Hz, 1H), 6.72-6.71 (m, 1H), 6.27 (s, 1H) ppm.

### Step (ii): Synthesis of N⁵-(3-(4-aminophenyl)propyl)-2-(furan-2-yl)-[1,2,4]triazolo[1,5-a]pyrimidine-5,7-diamine (66a)

A solution of intermediate 65 (0.3 g) in DMSO was taken in a sealed-tube fitted with Teflon® coated screw cap and 4-(3-aminopropyl)aniline (24a) (0.32 g, 1.92 mmol) and CsF was added to it. The resulting solution was sealed and stirred at 110° C. for 2 h. Upon completion of the reaction (as indicated by TLC), the reaction mixture was quenched with ice-water and the solid precipitate was filtered to provide the crude compound. It was then purified by combi-flash silica-gel chromatography using 70-100% EtOAc/hexane as eluent to obtain the title compound (0.3 g, 41%). LC-MS: m/z 350.1 (M+H⁺). ¹H NMR (400 MHz, DMSO-d₆): δ 7.82 (s, 1H), 7.18 (bs, 2H), 7.13 (s, 1H), 6.97 (d, J=2.4 Hz, 1H), 6.86 (d, J=8.4 Hz, 2H), 6.64 (s, 2H), 6.48 (d, J=8 Hz, 2H), 5.41 (s, 1H), 3.26-3.23 (m, 2H), 2.55-2.53 (m, 2H), 1.75-1.72 (m, 2H) ppm.

### Step (iii): Synthesis of 5-(3-(4-aminophenyl)propoxy)-2-(furan-2-yl)-[1,2,4]triazolo[1,5-a]pyrimidin-7-amine (66b)

To a stirred solution of 3-(4-aminophenyl)propan-1-ol (24b, 0.208 g, 1.38 mmol, 1.3 eq) in THF, NaH was added at 0° C. The mixture was allowed to stir for 10 mins and then intermediate 65 (0.25 g) was added under nitrogen atmosphere. The resultant mixture was heated at 70° C. for 16 h. Upon completion of the reaction (as indicated by TLC), water was added to the reaction mixture and extracted with EtOAc three times. The combined organic layer was washed with brine, dried over Na₂SO₄ and volatiles were removed under vacuum to provide the crude compound. It was then purified by combi-flash silica-gel chromatography using 45-50% EtOAc/Hexane as eluent to obtain the title compound (0.14 g, 38%). LCMS: m/z 351.1 (M+H⁺).

### Step (iii): Synthesis of N⁵-(3-(4-amino-2-fluorophenyl)propyl)-2-(furan-2-yl)-[1,2,4]triazolo[1,5-a]pyrimidine-5,7-diamine (66c)

The title compound was prepared by a reaction of intermediate 65 (0.2 g) and 4-(3-aminopropyl)-2-fluoroaniline (35a, 0.2 g, 0.84 mmol) by following the general procedure described above in step (ii); (0.09 g, crude); LCMS: m/z 368.1 (M+H⁺).

### Step (iv): Synthesis of N⁵-(3-(4-amino-3-fluorophenyl)propyl)-2-(furan-2-yl)-[1,2,4]triazolo[1,5-a]pyrimidine-5,7-diamine (66d)

The title compound was prepared by a reaction of intermediate 65 (0.3 g) 4-(3-aminopropyl)-3-fluoroaniline (35b, 0.32 g, 1.92 mmol) by following the following the general procedure described above in step (ii); (0.15 g, 31%). LCMS: m/z 368.30 (M+H⁺)

### Example A20: 5-(N-(4-(3-((7-amino-2-(furan-2-yl)-[1,2,4]triazolo[1,5-a]pyrimidin-5-yl)amino)propyl)phenyl)sulfamoyl)-3-chloro-2-hydroxybenzamide

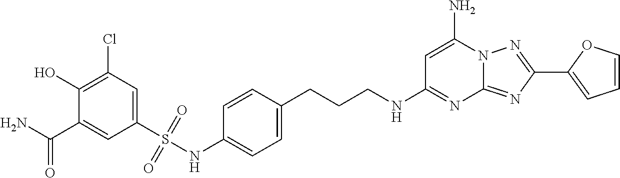 [SMILES: NC(=O)c1cc(S(=O)(=O)Nc2ccc(CCCNc3cc(N)n4nc(-c5ccco5)nc4n3)cc2)cc(Cl)c1O]

The title compound was prepared by a reaction of intermediate 66a (0.1 g) and 3-carbamoyl-4-hydroxybenzenesulfonyl chloride (4a, 0.077 g, 0.28 mmol) by following the general procedure described in step (ii) of Example A1 as white solid (0.005 g, 3%). LCMS: m/z 583.00 (M+H⁺); HPLC: 97.46%; ¹H NMR (400 MHz, DMSO-d₆): δ 15.05 (s, 1H), 10.02 (s, 1H), 8.27 (s, 1H), 7.82 (s, 2H), 7.20 (bs, 3H), 7.12-7.10 (m, 2H), 7.02-6.97 (m, 3H), 6.65-6.64 (m, 1H), 6.54 (s, 1H), 5.40 (s, 1H), 3.23-3.21 (m, 2H), 2.56-2.54 (m, 2H), 1.77-1.73 (m, 2H) ppm.

### Example A21: 5-(N-(4-(3-((7-amino-2-(furan-2-yl)-[1,2,4]triazolo[1,5-a]pyrimidin-5-yl)oxy)propyl)phenyl)sulfamoyl)-3-chloro-2-hydroxybenzamide

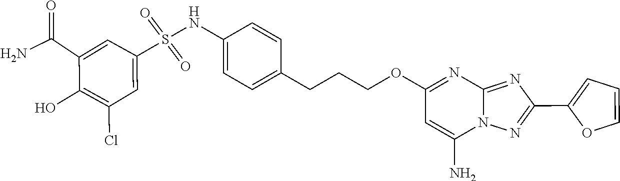 [SMILES: NC(=O)c1cc(S(=O)(=O)Nc2ccc(CCCOc3cc(N)n4nc(-c5ccco5)nc4n3)cc2)cc(Cl)c1O]

The title compound was prepared by a reaction of intermediate 66b (0.1 g) and 3-carbamoyl-4-hydroxybenzenesulfonyl chloride (4a, 0.13 g, 0.37 mmol) by following the general procedure described in step (ii) of Example A1 as white solid (0.003 g, 2%). LCMS: m/z 584.1 (M+H⁺); HPLC: 96.67%; ¹H NMR (400 MHz, DMSO-d₆): δ 10.58 (d, J=5.6 Hz, 1H), 9.60 (s, 1H), 8.42 (bs, 2H), 8.95 (s, 1H), 7.87-7.83 (m, 3H), 7.13-7.00 (m, 6H), 6.68 (s, 1H), 5.61 (s, 1H), 4.26-4.22 (m, 2H), 2.62-1.58 (m, 2H), 1.96-1.92 (m, 2H) ppm.

### Example A22: 5-(N-(4-(3-((7-amino-2-(furan-2-yl)-[1,2,4]triazolo[1,5-a]pyrimidin-5-yl)amino)propyl)-3-fluorophenyl)sulfamoyl)-3-chloro-2-hydroxybenzamide

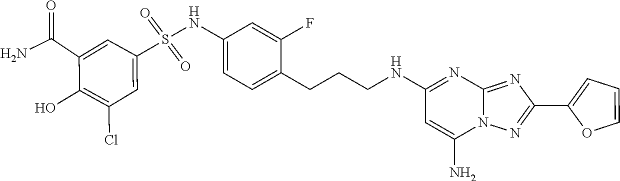 [SMILES: NC(=O)c1cc(S(=O)(=O)Nc2ccc(CCCNc3cc(N)n4nc(-c5ccco5)nc4n3)c(F)c2)cc(Cl)c1O]

The title compound was prepared by a reaction of intermediate 66c (0.09 g) and 3-carbamoyl-4-hydroxybenzenesulfonyl chloride (4a, 0.072 g, 0.26 mmol) by following the general procedure described in step (ii) of Example A1 as white solid (0.009 g, 6%). LCMS: m/z 601.10 (M+H⁺); HPLC: 99.67%; ¹H NMR (400 MHz, DMSO-d₆): δ 10.15 (s, 1H), 8.36-8.18 (m, 4H), 7.81 (s, 1H), 7.19 (m, 3H), 6.97-6.83 (m, 3H), 6.64 (s, 1H), 6.51 (s, 1H), 5.40 (s, 1H), 3.26-3.24 (m, 2H), 2.38-2.36 (m, 2H), 1.75-1.71 (m, 2H) ppm.

### Example A23: 5-(N-(4-(3-((7-amino-2-(furan-2-yl)-[1,2,4]triazolo[1,5-a]pyrimidin-5-yl)amino)propyl)-2-fluorophenyl)sulfamoyl)-3-chloro-2-hydroxybenzamide

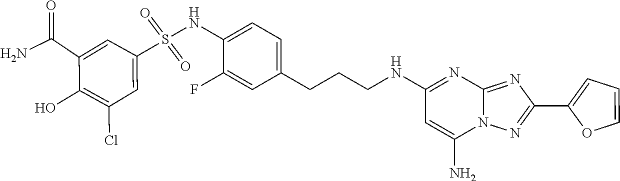 [SMILES: NC(=O)c1cc(S(=O)(=O)Nc2ccc(CCCNc3cc(N)n4nc(-c5ccco5)nc4n3)cc2F)cc(Cl)c1O]

The title compound was prepared by a reaction of intermediate 66d (0.15 g) and 3-carbamoyl-4-hydroxybenzenesulfonyl chloride (4a, 0.165 g, 0.61 mmol) by following the general procedure described in step (ii) of Example A1 as orange solid (0.017 g, 8%). LCMS: m/z 601.10 (M⁺); HPLC: 98.66%; ¹H NMR (400 MHz, DMSO-d₆): δ 15.09 (s, 1H), 9.94 (s, 1H), 9.03 (s, 1H), 8.35 (bs, 1H), 8.22 (s, 1H), 7.88-7.82 (m, 2H), 7.22-6.97 (m, 7H), 6.65 (s, 1H), 5.41 (s, 1H), 3.26-3.24 (m, 2H), 2.62-2.58 (m, 2H), 1.80-1.76 (m, 2H) ppm.

### Example E2: 5-(N-(6-(2-((7-amino-2-(furan-2-yl)-[1,2,4]triazolo[1,5-a]pyrimidin-5-yl)amino)ethyl)pyridin-3-yl)sulfamoyl)-3-chloro-2-hydroxybenzamide

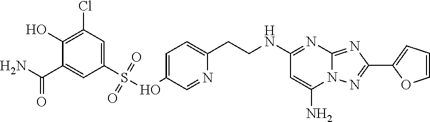 [SMILES: NC(=O)c1cc(S(=O)(=O)Oc2ccc(CCNc3cc(N)n4nc(-c5ccco5)nc4n3)nc2)cc(Cl)c1O]

### Step (i): Synthesis of N⁵-(2-(5-aminopyridin-2-yl)ethyl)-2-(furan-2-yl)-[1,2,4]triazolo[1,5-a]pyrimidine-5,7-diamine (67)

The title compound was prepared by a reaction of 5-chloro-2-(furan-2-yl)-[1,2,4]triazolo[1,5-a]pyrimidin-7-amine (65, 0.3 g) and intermediate 17 (0.21 g, 1.5 mmol) by following the general procedure described in step (ii) of preparation of Example A20-A23 (0.13 g, 31%). LCMS: m/z 337.1 (M+H⁺); ¹H NMR (400 MHz, DMSO-d₆): δ 7.88 (d, J=2.4 Hz, 1H), 7.83-7.82 (m, 1H), 7.21 (bs, 3H), 6.98-6.88 (m, 3H), 6.65-6.64 (m, 1H), 5.41 (s, 1H), 5.12 (s, 2H), 3.52-3.50 (m, 2H), 2.81-2.78 (m, 2H) ppm.

### Step (ii): Synthesis of 5-(N-(6-(2-((7-amino-2-(furan-2-yl)-[1,2,4]triazolo[1,5-a][1,3,5]triazin-5-yl)amino)ethyl)pyridin-3-yl)sulfamoyl)-3-chloro-2-hydroxybenzamide (Example E2)

The title compound was prepared by a reaction of intermediate 67 (0.13 g) and 3-carbamoyl-4-hydroxybenzenesulfonyl chloride (4a, 0.15 g, 0.57 mmol) by following the general procedure described in step (ii) of Example A1 as off-white solid (0.014 g, 6%). LCMS: m/z 570.1 (M⁺); HPLC: 98.34%; ¹H NMR (400 MHz, DMSO-d₆): δ 10.18 (s, 1H), 8.24 (bs, 2H), 7.82 (s, 2H), 7.44-7.41 (m, 1H), 7.20-7.17 (m, 4H), 6.98-6.97 (m, 1H), 6.64 (s, 1H), 5.39 (s, 1H), 3.56-3.54 (m, 2H), 2.92-2.89 (m, 2H) ppm.

### Example A24: 5-(N-(4-(3-((7-amino-2-(furan-2-yl)pyrazolo[1,5-a]pyrimidin-5-yl)amino)propyl)phenyl sulfamoyl)-3-chloro-2-hydroxybenzamide

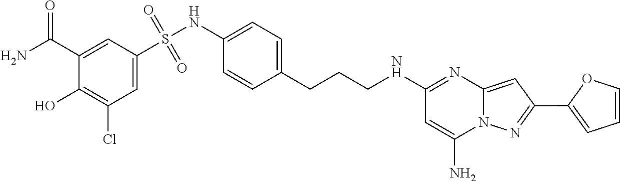 [SMILES: NC(=O)c1cc(S(=O)(=O)Nc2ccc(CCCNc3cc(N)n4nc(-c5ccco5)cc4n3)cc2)cc(Cl)c1O]

The compound, 5,7-dichloro-2-(furan-2-yl)pyrazolo[1,5-a]pyrimidine (intermediate 68) was prepared by following the procedure described in PCT publication WO2010074284.

### Step (i): Synthesis of 5-chloro-2-(furan-2-yl)pyrazolo[1,5-a]pyrimidin-7-amine (69)

The title compound was prepared from intermediate 68 (0.31 g, 1.22 mmol) by following the procedure described in step (i) of preparation of examples A20 to A23. Yield: 0.25 g, 87%. LCMS: m/z 235.1 (M+H⁺); ¹H NMR (400 MHz, DMSO-d₆): δ 8.11 (bs, 2H), 7.81 (s, 1H), 7.01 (d, J=2.8 Hz, 1H), 6.67-6.64 (m, 2H), 6.06 (s, 1H).

### Step (ii): Synthesis of N⁵-(3-(4-aminophenyl)propyl)-2-(furan-2-yl)pyrazolo[1,5-a]pyrimidine-5,7-diamine (70)

The title compound was prepared by a reaction of 5-chloro-2-(furan-2-yl)pyrazolo[1,5-a]pyrimidin-7-amine (69, 0.15 g) and 4-(3-aminopropyl)aniline (24a, 0.143 g, 0.95 mmol) by following the general procedure described in step (ii) of preparation of Example A20-A23. Yield: 0.07 g, 31%. LCMS: m/z 349.2 (M+H⁺); ¹H NMR (400 MHz, DMSO-d₆): δ 7.73 (bs, 2H), 6.87-6.73 (s, 5H), 6.59 (s, 1H), 6.48 (d, J=8 Hz, 2H), 6.07 (s, 1H), 5.33 (s, 1H), 4.79 (s, 2H), 3.21-3.16 (m, 2H), 2.67-2.62 (m, 2H), 1.74-1.71 (m, 2H) ppm.

### Step (iii): Synthesis of 5-(N-(4-(3-((7-amino-2-(furan-2-yl)pyrazolo[1,5-a]pyrimidin-5-yl)amino)propyl)phenyl)sulfamoyl)-3-chloro-2-hydroxybenzamide (Example A24)

The title compound was prepared by a reaction of intermediate 70 (0.07 g) and 3-carbamoyl-4-hydroxybenzenesulfonyl chloride (4a, 0.065 g, 0.24 mmol) by following the general procedure described step (ii) of Example A1: (Scheme 1) as white solid (0.013 g, 11%). LCMS: m/z 582.15 (M+H⁺); HPLC: 99.51%; ¹H NMR (400 MHz, DMSO-d₆): δ 15.01 (s, 1H), 12.83 (s, 1H), 9.91 (s, 1H), 8.21-8.13 (m, 2H), 7.75 (s, 2H), 7.12-6.81 (m, 8H), 6.60-6.59 (m, 1H), 6.09 (s, 1H), 5.32 (s, 1H), 3.21-3.17 (m, 2H), 2.57-2.55 (m, 2H), 1.75-1.72 (m, 2H) ppm.

### Example A25: Methyl ((4-(N-(4-(3-((7-amino-2-(furan-2-yl)-[1,2,4]triazolo[1,5-a][1,3,5]triazin-5-yl)amino)propyl)phenyl)sulfamoyl)-2-carbamoyl-6-chlorophenoxy)carbonyl)valinate

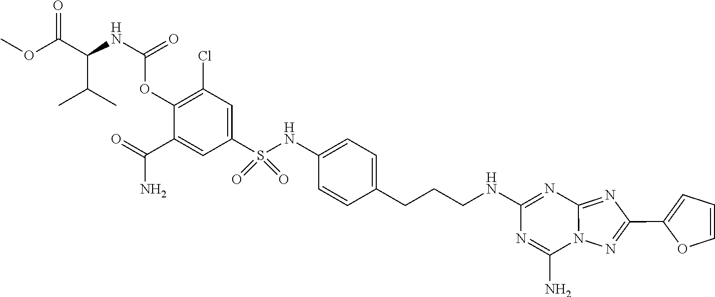 [SMILES: COC(=O)[C@@H](NC(=O)Oc1c(Cl)cc(S(=O)(=O)Nc2ccc(CCCNc3nc(N)n4nc(-c5ccco5)nc4n3)cc2)cc1C(N)=O)C(C)C]

### Step (i): Synthesis of tert-butyl (3-(4-((3-carbamoyl-5-chloro-4-hydroxyphenyl)sulfonamido)phenyl)propyl)carbamate (71)

To a stirred solution of tert-butyl (3-(4-aminophenyl)propyl)carbamate (prepared according to procedure described in WO2014180524) (4.28 g, 17.1 mmol, 1.1 eq) in THF, pyridine (6.45 g, 5.3 eq) at 0° C. It was stirred for 10 min, then 3-carbamoyl-5-chloro-4-hydroxybenzenesulfonyl chloride (4a, 4.2 g) was added. The resultant mixture was slowly warmed to RT and stirred for 16 h. Upon completion of the reaction as indicated by TLC, the reaction mixture was quenched with cooled citric acid solution and extracted with ethyl acetate. The volatiles were removed under vacuum to obtain the title compound which was used without any further purification (4.5 g, 60%). LCMS: m/z 384.0 (M−COOtBu+); HPLC: 95.22%;

¹H NMR (400 MHz, DMSO-d₆): δ 15.02 (s, 1H), 10.04 (s, 1H), 9.01 (bs, 1H), 8.50 (bs, 1H), 8.30 (s, 1H), 7.85 (s, 1H), 7.07 (d, J=8.4 Hz, 2H), 6.99 (d, J=8 Hz, 2H), 6.90 (s, 1H), 2.87-2.86 (s, 2H), 2.44-2.42 (m, 2H), 1.59-1.57 (m, 2H), 1.36 (s, 9H) ppm.

### Step (ii): Synthesis methyl (chlorocarbonyl)valinate (72)

To a solution of triphosgene (0.26 g, 0.5 eq) in dry DCM, pyridine (0.56 g, 4 eq) was added dropwise. The resulting mixture was stirred at 0° C. for 15 min. Then, methyl L-valinate hydrochloride was added and the resulting mixture was stirred at RT for 2 h. Upon completion of the reaction, the reaction mixture was quenched with 1N HCl (0.1 mL) and then diluted with DCM. The organic layer was washed with brine and concentrated under vacuum to provide the crude which was used as such for the next step (0.32 g, 92%).

### Step (iii): Synthesis of methyl ((4-(N-(4-(3-((tert-butoxycarbonyl)amino)propyl)phenyl)sulfamoyl)-2-carbamoyl-6-chlorophenoxy)carbonyl)valinate (73)

To a stirred solution of intermediate 71 (0.6 g) in DCM, triethyl amine (0.37 g, 3 eq) and DMAP (0.046 g, 0.3 eq) were added at 0° C. under argon atmosphere. After 30 mins of stirring, in a solution of intermediate 72 (0.3 g, 1.25 eq) DCM was added dropwise to the reaction mixture. The reaction was stirred at room temperature for 16 h. Upon completion of the reaction, the volatiles were removed under vacuum to provide the crude. The desired product was isolated through silica gel flash chromatography (0.33 g, 42%). LC-MS: m/z 641.2 (M+).

### Step (iv): Synthesis of methyl ((4-(N-(4-(3-aminopropyl)phenyl)sulfamoyl)-2-carbamoyl-6-chlorophenoxy)carbonyl)valinate (74)

To a stirred solution of intermediate 73 (0.4 g) in 1,4-dioxane (4 mL) was added 4M HCl in dioxane (2 mL) at RT. The resulting reaction mixture was stirred at RT for 1 h. Upon completion of the reaction (as indicated by TLC), the reaction mixture was concentrated under vacuum and washed with ether and pentane to provide the crude. It was used in next step without any further purification (0.120 g, crude). LC-MS: m/z 541.0 (M+).

### Step (iv): Synthesis of methyl ((4-(N-(4-(3-((7-amino-2-(furan-2-yl)-[1,2,4]triazolo[1,5-a][1,3,5]triazin-5-yl)amino)propyl)phenyl)sulfamoyl)-2-carbamoyl-6-chlorophenoxy)carbonyl)valinate (Example A25)

To a stirred solution of intermediate 1 (0.156 g) and intermediate 74 (0.28 g, 1 eq) in DMSO: THE (1:1, 4 mL), triethylamine (0.105 g, 2 eq) was added dropwise at 0° C. The reaction was stirred at RT for 5 h. Upon completion of the reaction (as indicated by TLC), ice chips were added to the reaction mixture. The off-white solid precipitate was filtered and dried to provide the crude. The crude material was purified using preparative HPLC to obtain the title compound as white solid (0.015 g, 3.9%). LCMS: m/z 740.95 (M+); HPLC: 97.55%;

¹H NMR (400 MHz, DMSO-d₆): δ9.08 (s, 1H), 8.44 (bs, 2H), 8.30 (s, 1H), 8.18 (bs, 2H), 7.94-7.88 (m, 2H), 7.62-7.60 (m, 1H), 7.34 (d, J=8 Hz, 2H), 7.15 (d, J=7.6 Hz, 2H), 7.08-7.05 (m, 1H), 6.97 (d, J=8.4 Hz, 1H), 6.69-6.67 (m, 1H), 3.97 (dd, J=7.6 Hz, 6.8 Hz, 1H), 3.59 (s, 1H), 3.32-3.30 (m, 2H), 2.71-2.67 (s, 2H), 2.00-1.96 (m, 1H), 1.87-1.84 (m, 2H), 0.75 (d, J=6.4 Hz, 3H), 0.68 (d, J=6.4 Hz, 3H) ppm.

### Example A26: 5-(N-(4-(3-((7-amino-2-(furan-2-yl)-[1,2,4]triazolo[1,5-a][1,3,5]triazin-5-yl)amino)propyl)phenyl)sulfamoyl)-3-chloro-2-hydroxybenzamide sodium salt

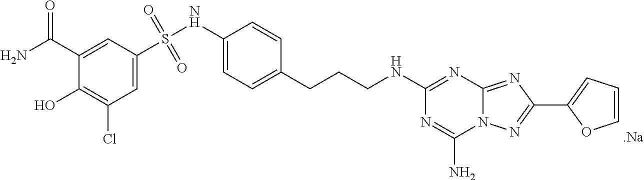

To a stirred solution of 5-(N-(4-(3-((7-amino-2-(furan-2-yl)-[1,2,4]triazolo[1,5-a][1,3,5]triazin-5-yl)amino)propyl)phenyl)sulfamoyl)-3-chloro-2-hydroxybenzamide (All, 0.04 g) in MeOH (0.5 mL), a solution of NaOH (0.006 g) in water (1 mL) was added dropwise. The reaction was allowed to stir at RT for 15 min. The reaction mass was directly lyophilized to give the title product as white solid (0.02 g, 48.54%). LCMS: m/z 584.45 (M+); HPLC: 95.20%; ¹H NMR (400 MHz, DMSO-d₆): δ 11.05 (s, 1H), 7.97 (s, 1H), 7.82 (s, 1H), 7.36 (s, 1H), 7.00 (s, 1H), 6.67-6.59 (m, 5H), 3.20-3.18 (m, 2H), 2.36-2.34 (m, 2H), 1.70-1.68 (m, 2H) ppm.

### Example A27: Ethyl (5-((3-(4-((3-chloro-N-(ethoxycarbonyl)-5-((ethoxycarbonyl)carbamoyl)-4-((ethoxycarbonyl)oxy)phenyl)sulfonamido)phenyl)propyl)(ethoxycarbonyl)amino)-2-(furan-2-yl)-[1,2,4]triazolo[1,5-a][1,3,5]triazin-7-yl)(ethoxycarbonyl)carbamate

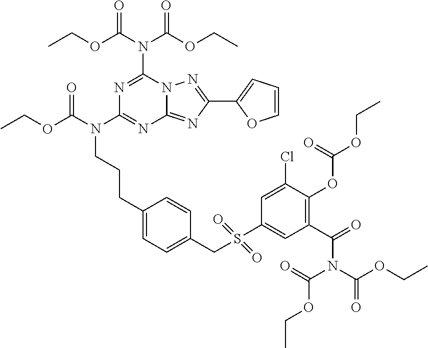 [SMILES: CCOC(=O)Oc1c(Cl)cc(S(=O)(=O)Cc2ccc(CCCN(C(=O)OCC)c3nc(N(C(=O)OCC)C(=O)OCC)n4nc(-c5ccco5)nc4n3)cc2)cc1C(=O)N(C(=O)OCC)C(=O)OCC]

To a stirred solution of Example A11 (0.3 g, 0.514 mmol) in THF, triethyl amine (1.67 g, 30 eq) and ethyl chloroformate (2.08 g, 40 eq) were added at 0° C. The resultant mixture was slowly warmed to RT and stirred for 48 h. Upon completion of the reaction as indicated by TLC, the reaction mixture was quenched with water and extracted with ethyl acetate. The organic layer was washed with brine, dried over Na₂SO₄ and volatiles were removed under vacuum to provide the crude which was then purified using preparative HPLC. The title compound was obtained as white solid (35 mg, 6.70%). LCMS: m/z 1016.7 (M⁺); HPLC: 95.00%.

### Example A28: Tert-Butyl (1-(5-(N-(4-(3-((7-amino-2-(furan-2-yl)-[1,2,4]triazolo[1,5-a][1,3,5]triazin-5-yl)amino)propyl)phenyl)sulfamoyl)-3-chloro-2-hydroxybenzamido)-3-methyl-1-oxobutan-2-yl)carbamate

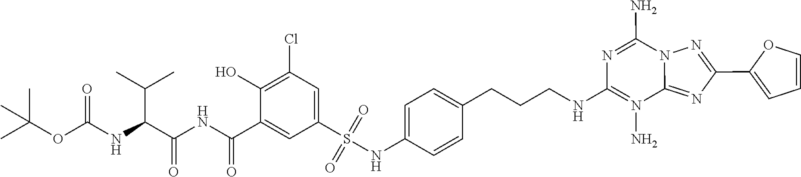

To a stirred solution of Example-A11 (0.025 g, 0.043 mmol), L-Boc valine (0.04 g, 30 eq), EDC.HCl (0.02 g, 1.45 eq), HOBt (0.009 g, 1.55 eq) in THF, DMAP (0.008 g, 1.5 eq) was added. The resultant mixture was heated at 75° C. for 16 h. Upon completion of the reaction as indicated by TLC, volatiles were removed under vacuum. To the resulting crude, ice-water was added and the solid precipitate was filtered and washed with ether and pentane. The crude was then purified using preparative HPLC to obtain the tittle compound as white solid (12 mg, 35.63%). LCMS: m/z 783.0 (M⁺); HPLC: 97.22%.

### General Procedure for the Preparation of Examples A29-A31

To a stirred solution of Example A11 in DMF, potassium carbonate (10 eq) and respective phenyl carbonate (1.5 eq) were added. The resultant mixture was heated at 90° C. for 18 h. Upon completion of the reaction as indicated by TLC, DMF was removed under vacuum. The crude mass was washed with hexane, pentane and ether. It was then purified by preparative HPLC.

### Example A29: Hexyl (7-amino-2-(furan-2-yl)-[1,2,4]triazolo[1,5-a][1,3,5]triazin-5-yl)(3-(4-((3-carbamoyl-5-chloro-4-hydroxyphenyl)sulfonamido)phenyl)propyl)carbamate

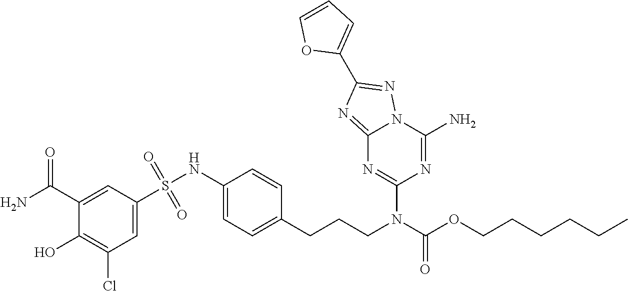 [SMILES: CCCCCCOC(=O)N(CCCc1ccc(NS(=O)(=O)c2cc(Cl)c(O)c(C(N)=O)c2)cc1)c1nc(N)n2nc(-c3ccco3)nc2n1]

The title compound was prepared by a reaction of Example A11 (0.1 g, 0.171 mmol) and hexyl (4-nitrophenyl) carbonate (prepared following literature procedure as described in WO2015128875) (0.069 g, 0.257 mmol) by following the general procedure described in the preparation of Examples A29-A31 as off-white solid (62 mg, 50.91%). LCMS: m/z 712.40 (M+); HPLC: 97.92%.

### Example A30: 3-(Hexadecyloxy)propyl (7-amino-2-(furan-2-yl)-[1,2,4]triazolo[1,5-a][1,3,5]triazin-5-yl)(3-(4-((3-carbamoyl-5-chloro-4-hydroxyphenyl)sulfonamido)phenyl)propyl)carbamate

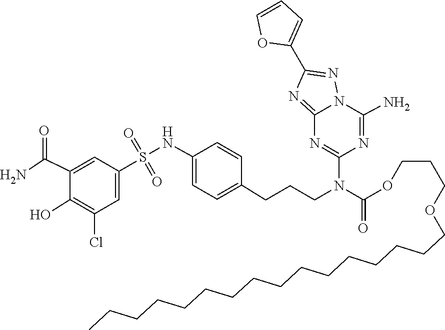 [SMILES: CCCCCCCCCCCCCCCCOCCCOC(=O)N(CCCc1ccc(NS(=O)(=O)c2cc(Cl)c(O)c(C(N)=O)c2)cc1)c1nc(N)n2nc(-c3ccco3)nc2n1]

The title compound was prepared by a reaction of Example A11 (0.15 g, 0.257 mmol) and 3-(hexadecyloxy)propyl (4-nitrophenyl) carbonate (prepared according to similar procedure as described in WO2017161071) (0.18 g, 0.386 mmol) by following the general procedure described in the preparation of Examples A29-A31 as off-white solid (130 mg, 55.55%). LCMS: m/z 910.60 (M⁺); HPLC: 95.50%.

### Example A31: 1-(((7-Amino-2-(furan-2-yl)-[1,2,4]triazolo[1,5-a][1,3,5]triazin-5-yl)(3-(4-((3-carbamoyl-5-chloro-4-hydroxyphenyl)sulfonamido)phenyl)propyl)carbamoyl)oxy)ethyl 3-methylbutanoate

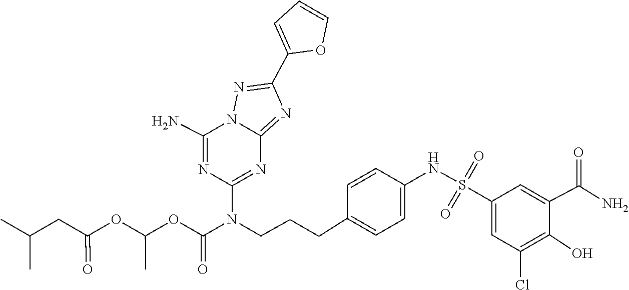 [SMILES: CC(C)CC(=O)OC(C)OC(=O)N(CCCc1ccc(NS(=O)(=O)c2cc(Cl)c(O)c(C(N)=O)c2)cc1)c1nc(N)n2nc(-c3ccco3)nc2n1]

The title compound was prepared by a reaction of Example A11 (0.15 g, 0.257 mmol) and 1-(((2-fluorophenoxy)carbonyl)oxy)ethyl 3-methylbutanoate (prepared according to similar procedure as described in US20140243544) (0.11 g, 0.386 mmol) by following the general procedure described in the preparation of Examples A29-A31 as off-white solid (35 mg, 18.01%). LCMS: m/z 670.03 (M-COⁱBu⁺); HPLC: 92.63%.

### Example P1: CD73 Biochemical Assay

A colorimetric-based method was used for assaying compounds of the invention for CD73 enzyme inhibitory activity. Human CD73 catalyzes the conversion of AMP to adenosine with the release of orthophosphate. A Malachite Green Phosphate Detection kit is used to measure the formation of orthophosphate product. Typically, 40 μL of human-CD73 (Trp27-Lys547, His-tag) in assay buffer (20 mM Tris pH 7.5, 5 mM MgCl₂) is added to a 96-well plate containing 20 μl of test compound in final 1% DMSO, serially diluted in 1:2 in an 10-12 point titration. A compound of the present invention and enzyme are incubated for 30 minutes at room temperature. Next, 20 μL of AMP in assay buffer is added to the plate. The final concentration of CD73 and AMP are 2 nM and 50 μM respectively. Following 30 minutes reaction at room temperature, 20 μL of Malachite reagent is added to all the reaction wells. The formation of green complex formed between Malachite Green, molybdate and free orthophosphate is measured on a plate reader (620 nm). The activity of the test compound on inhibition of CD73 is expressed as percent inhibition of internal assay controls as presented in Table-1A below and a four-parameter curve fit is applied in Graph-pad Prism® to determine the potency of the compounds, presented in Table-1B.

TABLE 1A
Percent inhibition data in CD73

| Example | % inhibition at 20 μM | Example | % inhibition at 20 μM |
|---|---|---|---|
| A1 | 91 | A14 | 94 |
| A2 | 97 | A15 | 96 |
| A3 | 96 | A16 | 63 |
| A4 | 27 | A17 | 95 |
| A5 | 99 | C1 | 100 |
| A6 | 98 | C2 | 96 |
| A7 | 42 | D1 | 49 |
| A8 | 70 | D2 | 62 |
| A9 | 39 | A18 | 87 |
| E1 | 86 | A19 | 96 |
| B1 | 35 | A20 | 92 |
| B2 | 39 | A21 | 98 |
| A10 | 17 | A22 | 95 |
| B3 | 9 | A23 | 95 |
| B4 | 99 | E2 | 94 |
| A11 | 98 | A24 | 92 |
| A12 | 99 | A25 | 99 |
| A13 | 97 | A26 | 96 |

TABLE 1B
Potency (IC₅₀ values) of selected compounds in CD73

| Group | Examples |
|---|---|
| A | A11, A12, A13, A14, A20, A22, A23, A24, A25 and A26 |
| B | A2, A3, A5, A6, A15, A17, C1, C2, D1, A21 and E2 |
| C | A1, E1, D2, A18 and A19 |

The IC₅₀ values for selected compounds are summarized in the table given below wherein group “A” refers to compounds with IC₅₀ values lower than 0.1 μM, group “B” refers to compounds with IC₅₀ values between 0.11 μM and 1.0 μM (both inclusive) and group “C” refers to compounds with IC₅₀ values higher than 1.0 μM.

### Example P2: A2aR Biochemical Assay

The compounds of present invention were assayed for A2aR enzyme binding activity in a TR-FRET based method. Typically, 10 μL of A2aR labelled cells in assay buffer (Tag-lite, 1×) is added to a 384-well plate containing 5 μL of a compound of present invention in final 1% DMSO, serially diluted in 1:5 in a 5-6 point titration. The aforesaid compound and cells are incubated for 30 minutes at room temperature. Next, 5 μL of A2aR antagonist (fluorescent ligand) in assay buffer is added to the plate. The final concentration of A2aR antagonist is 10 nM. Following 30 minutes reaction at room temperature, the plate was read on a plate reader (Ex: 340 nm Em: 615 nm and 665 nm) to measure the binding of test compound to A2aR cells. The results are interpreted as percentage inhibition as presented in Table-2A of binding by the test compound with respect to the DMSO control. A four-parameter curve fit is applied in Graph-pad Prism® to determine the potency of the compounds, presented in Table-2B.

TABLE 2A
Percent inhibition data in A2aR

| Example | % inhibition at 20 μM | Example | % inhibition at 20 μM |
|---|---|---|---|
| A1 | 78 | A10 | 88 |
| A2 | 80 | B3 | 88 |
| A3 | 83 | B4 | 86 |
| A4 | 81 | A11 | 88 |
| A5 | 84 | A12 | 72 |
| A6 | 79 | A13 | 83 |
| A7 | 84 | A14 | 82 |
| A8 | 81 | A15 | 83 |
| A9 | 82 | A16 | 83 |
| E1 | 83 | A17 | 82 |
| B1 | 80 | C1 | 81 |
| B2 | 79 | C2 | 85 |
| D1 | 85 | A22 | 81 |
| D2 | 79 | A23 | 78 |
| A18 | 82 | E2 | 85 |
| A19 | 83 | A24 | 79 |
| A20 | 80 | A25 | 81 |
| A21 | 82 | A26 | 80 |

TABLE 1B
Potency (IC₅₀ values) of selected compounds in A2aR

| Group | Examples |
|---|---|
| A | A1, A3, A5, A11 and C1 |
| B | A13 and A20 |
| C | A2, A12, A23 and A24 |

The IC₅₀ values for selected compounds are summarized in the table given below wherein group “A” refers to compounds with IC₅₀ values lower than 0.025 μM, group “B” refers to compounds with IC₅₀ value between 0.0251 μM and 0.05 μM (both inclusive) and group “C” refers to compounds with IC₅₀ value higher than 0.05 μM.

We claim:

1. A compound of formula (I) 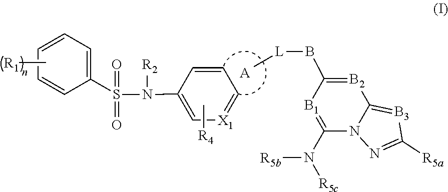
or a pharmaceutically acceptable salt or a stereoisomer or a pro rug thereof;
wherein,
X₁ is C or N;
A is optionally fused 5- to 6-membered heteroaryl ring containing 1 or 2 heteroatoms selected from N, O and S; wherein if A is absent, L is attached with 6-membered ring containing X₁;
L represents alkylene, alkenylene or alkynylene, wherein one or more C atoms are replaced with N or O; and each of alkylene, alkenylene and alkynylene is optionally substituted with one, two or three substituents selected from halo, hydroxyl, haloalkyl, amino, amido, alkyl, aryl, cycloalkyl, heteroaryl and heterocycloalkyl;
B represents —O— or —NR_{5d}—;
each B₁, B₂ and B₃ independently represents —N— or —CX₂—;
X₂ represents hydrogen, alkyl, cycloalkyl, aryl, 5- or 6-membered heterocycloalkyl or 5- or 6-membered heteroaryl;
R₁ at each occurrence independently represents alkyl, —NRₐR_{b}, halo, haloalkyl, —CONRₐR_{b}, —ORₐ, cycloalkyl, aryl, heteroaryl or heterocycloalkyl; wherein each of cycloalkyl, aryl, heteroaryl and heterocycloalkyl is substituted with one, two or three occurrences of R₃;
alternatively, any two R₁ groups, bonded to adjacent carbon atoms, combine together to form a 5- or 6-membered heterocycloalkyl ring containing 1 or 2 heteroatoms selected from N, O and S; wherein the said heterocycloalkyl is substituted with one, two or three occurrences of R₃;
R₂ represents hydrogen, halo, alkyl, hydroxyl or cycloalkyl;
R₃ represents hydrogen, oxo, halo, amino, alkyl, amido, hydroxyl, cycloalkyl, aryl, heteroaryl or heterocycloalkyl;
R₄ represents hydrogen, hydroxyl, halo or alkyl;
Rₐ and R_{b}, each independently represents hydrogen, alkyl, haloalkyl, ester, —COO-alkyl, Aaa or —CO-Aaa; wherein 1 or 2 C atoms in the said alkyl chain are optionally replaced with O; and the said alkyl is optionally substituted with alkoxy or oxo;
Aaa is an amino acid residue selected from Ala, Ser, Thr, Cys, Val, Leu and Ile; wherein the C-terminus thereof is a free terminus, is amidated or is esterified; and N-terminus thereof is a free terminus or Boc-protected;
R₅ₐ is aryl or 5- or 6-membered heteroaryl;
R_{5b}, R_{5c} and R_{5d} each independently represents hydrogen, alkyl, acyl, ester, —COO-alkyl, cycloalkyl, aryl, aralkyl, heterocycloalkyl, heteroaryl or heteroaralkyl; wherein 1 or 2 C atoms in the said alkyl chain are optionally replaced with O; and the said alkyl is optionally substituted with alkoxy or oxo;
‘n’ is an integer selected from 0, 1, 2, 3 and 4.

2. (canceled)

3. The compound of claim 1, wherein L represents (C₁-C₆)alkylene, (C₂-C₆)alkenylene or (C₂-C₆)alkynylene, wherein one or more C atoms are replaced with N or O atom.

4. The compound of claim 1, wherein R₁, at each occurrence, represents alkyl, halo, haloalkyl, —CONH₂, —OH or —OCONHCH[CH(CH₃)₂]COOCH₃; or any two R₁ groups, bonded to adjacent carbon atoms, combine together to form

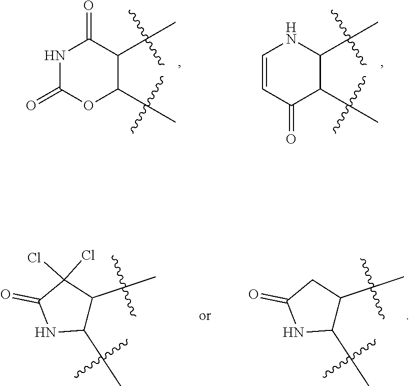 [SMILES: *C1OC(=O)NC(=O)C1* ; *C1NC=CC(=O)C1* ; *C1NC(=O)C(Cl)(Cl)C1* ; *C1CC(=O)NC1*]

5. The compound of claim 1, wherein, A is fused 5-membered heteroaryl ring selected from furan, thiophene, pyrrole, pyrazole, imidazole, oxazole, isoxazole, thiazole and isothiazole.

6. The compound of claim 1, wherein the group 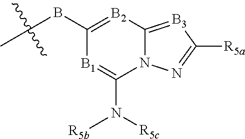
represents:

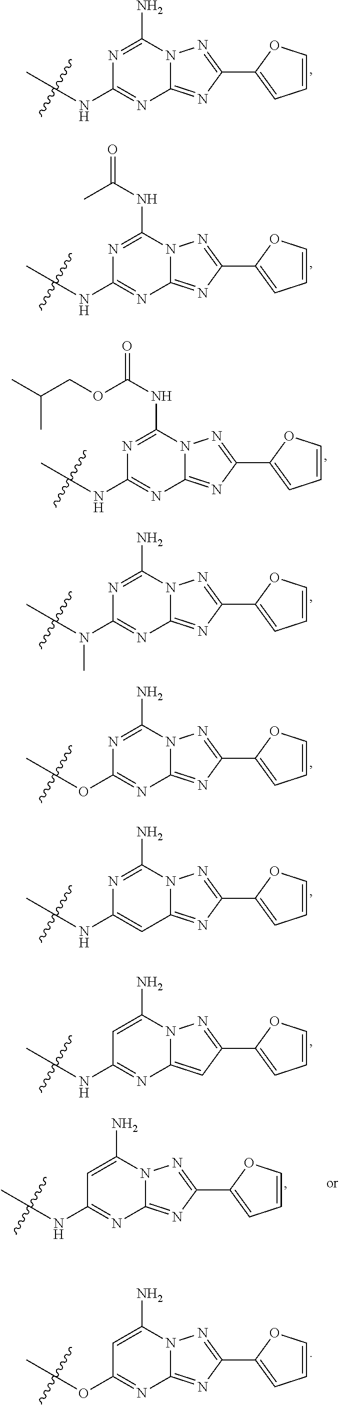

7. (canceled)

8. The compound of claim 1, represented by formula (IA):

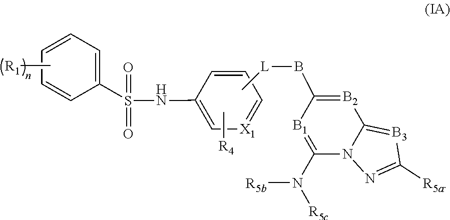

9.-13. (canceled)

14. The compound of claim 8, wherein;
R₁, at each occurrence, independently represents —CH₃, —F, —Cl, —CF₃, —CONH₂, —OH or —OCONHCH[CH(CH₃)₂]COOCH₃; or any two R₁ groups, bonded to adjacent carbon atoms, combine together to form 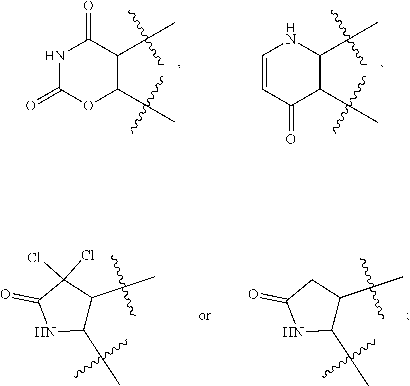 [SMILES: *C1OC(=O)NC(=O)C1* ; *C1NC=CC(=O)C1* ; *C1NC(=O)C(Cl)(Cl)C1* ; *C1CC(=O)NC1*]
R₄ represents hydrogen, hydroxyl, —F or —Cl;
X₁ is C or N;
L represents —CH₂—, —(CH₂—CH₂)—, —(CH₂—CH₂—CH₂)—, —(CH₂—CH₂—CH₂—CH₂)— or —NH(CH₂—CH₂)—;
B represents —O—, —NH— or —N(CH₃)—;
B₁, B₂ and B₃ independently represents —N— or —CH—;
R₅ₐ is furanyl, thienyl, pyrrolyl, pyrazolyl, imidazolyl, oxazolyl, isoxazolyl, thiazolyl, isothiazolyl, 1H-tetrazolyl, oxadiazolyl or triazolyl;
R_{5b} represents hydrogen, acyl or —COO-alkyl;
R_{5c} is hydrogen; and
‘n’ is an integer selected from 0, 1, 2 and 3.

15. The compound of claim 1, represented by formula (IB):

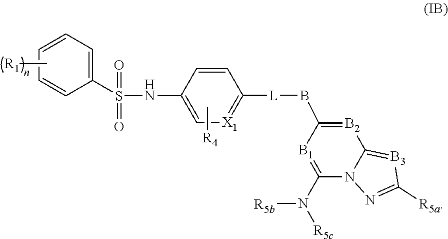

16.-21. (canceled)

22. The compound of claim 15, wherein,
L represents (C₁-C₆)alkylene or (C₂-C₆)alkenylene wherein one or more C atoms of (C₁-C₆)alkylene or (C₂-C₆)alkenylene groups are replaced with N or O atom;
B represents —O— or —NH—;
B₁ and B₃ each independently represents —N— or —CX₂—; B₂ represents —N—;
X₂ represents hydrogen or alkyl;
R₁ at each occurrence independently represents alkyl, —NH₂, halo, haloalkyl, —CONH₂ or —OH; or
any two R₁ groups, bonded to adjacent carbon atoms, combine together to form a 5- or 6-membered heterocycloalkyl ring containing 1 or 2 heteroatoms selected from N, O and S; wherein the said heterocycloalkyl is substituted with one, two or three occurrences of R₃;
R₃ represents hydrogen, oxo, halo, hydroxyl, cycloalkyl, aryl or 5- or 6-membered heteroaryl;
R₄ is hydrogen, hydroxyl or halo;
R₅ₐ is 5- or 6-membered heteroaryl;
R_{5b} and R_{5c} each independently represents hydrogen, alkyl, acyl, ester, —COO-alkyl, cycloalkyl, phenyl, aralkyl, 5- to 6-membered heterocycloalkyl to 5- to 6-membered heteroaryl;
‘n’ is an integer selected from 1, 2 and 3.

23. (canceled)

24. The compound of claim 1, represented by formula (IC):

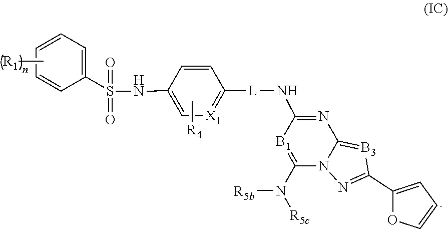

25.-28. (canceled)

29. The compound of claim 24, wherein
R₁, at each occurrence, independently represents —CH₃, —F, —Cl, —CF₃, —CONH₂, —OH or —OCONHCH[CH(CH₃)₂]COOCH₃; or any two R₁ groups, bonded to adjacent carbon atoms, combine together to form 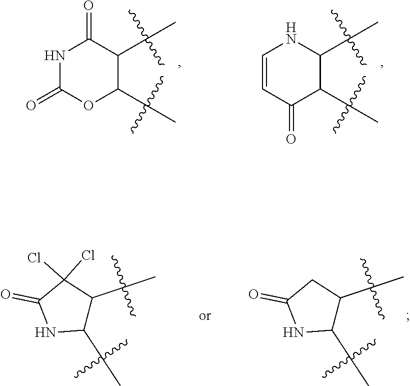 [SMILES: *C1OC(=O)NC(=O)C1* ; *C1NC=CC(=O)C1* ; *C1NC(=O)C(Cl)(Cl)C1* ; *C1CC(=O)NC1*]
R₄ represents hydrogen, hydroxyl, —F or —Cl;
L represents —CH₂—, —(CH₂—CH₂)—, —(CH₂—CH₂—CH₂)— or —(CH₂—CH₂—CH₂—CH₂)—;
X₁ is C or N;
B₁ and B₃ independently represents —N— or —CH—;
R_{5b} represents hydrogen, acyl or —COO-alkyl;
R_{5c} is hydrogen; and
‘n’ is an integer selected from 0, 1, 2 and 3.

30. The compound of claim 1, represented by formula (ID).

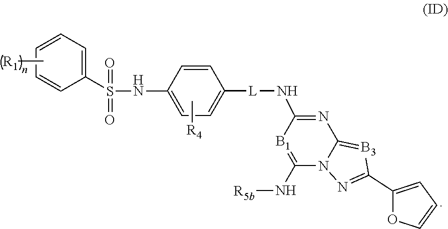

31. The compound of claim 30, wherein
R₁, at each occurrence, independently represents —CH₃, —F, —Cl, —CF₃, —CONH₂, —OH or —OCONHCH[CH(CH₃)₂]COOCH₃; or any two R₁ groups, bonded to adjacent carbon atoms, combine together to form 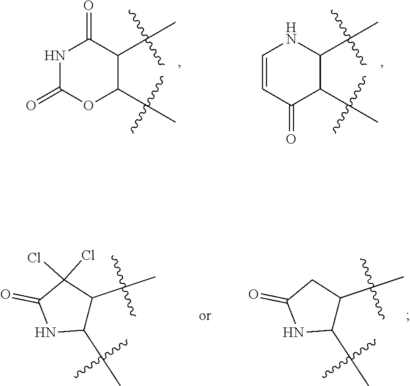 [SMILES: *C1OC(=O)NC(=O)C1* ; *C1NC=CC(=O)C1* ; *C1NC(=O)C(Cl)(Cl)C1* ; *C1CC(=O)NC1*]
R₄ represents hydrogen, hydroxyl, —F or —Cl;
L represents —CH₂—, —(CH₂—CH₂)—, —(CH₂—CH₂—CH₂)— or —(CH₂—CH₂—CH₂—CH₂)—;
B₁ and B₃ independently represents —N— or —CH—;
R_{5b} represents hydrogen, acyl or —COOCH₂CH(CH₃)₂; and
‘n’ is an integer selected from 0, 1, 2 and 3.

32. The compound of claim 1, represented by formula (IE) or (IF);

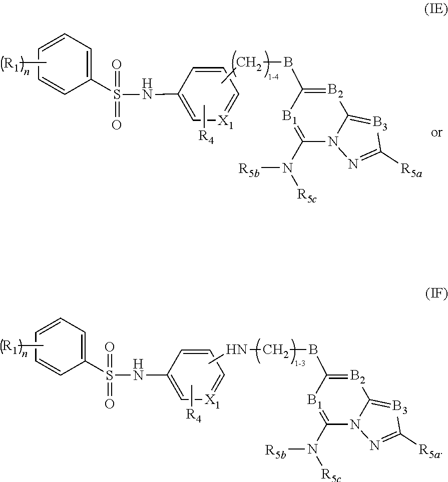

33. The compound of claim 32, wherein
R₁, at each occurrence, independently represents —CH₃, —F, —Cl, —CF₃, —CONH₂, —OH or —OCONHCH[CH(CH₃)₂]COOCH₃; or any two R₁ groups, bonded to adjacent carbon atoms, combine together to form 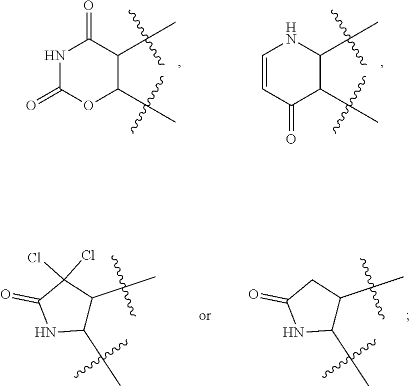 [SMILES: *C1OC(=O)NC(=O)C1* ; *C1NC=CC(=O)C1* ; *C1NC(=O)C(Cl)(Cl)C1* ; *C1CC(=O)NC1*]
R₄ represents hydrogen, hydroxyl, —F or —Cl;
X₁ is C or N;
B represents —O—, —NH— or —N(CH₃)—;
B₁, B₂ and B₃ independently represents —N— or —CH—;
R₅ₐ is furanyl, thienyl, pyrrolyl, pyrazolyl, imidazolyl, oxazolyl, isoxazolyl, thiazolyl, isothiazolyl, 1H-tetrazolyl, oxadiazolyl or triazolyl;
R_{5b} represents hydrogen, acyl or —COOalkyl-;
R_{5c} is hydrogen; and
‘n’ is an integer selected from 0, 1, 2 and 3.

34. The compound of claim 1, selected from:

| Example | IUPAC name |
|---|---|
| A1 | 5-(N-(3-(((7-amino-2-(furan-2-yl)-[1,2,4]triazolo[1,5-a][1,3,5]triazin-5- |
| | yl)amino)methyl)phenyl)sulfamoyl)-3-chloro-2-hydroxybenzamide; |
| A2 | 5-(N-(4-(((7-amino-2-(furan-2-yl)-[1,2,4]triazolo[1,5-a][1,3,5]triazin-5- |
| | yl)amino)methyl)phenyl)sulfamoyl)-3-chloro-2-hydroxybenzamide; |
| A3 | 5-(N-(4-(2-((7-amino-2-(furan-2-yl)-[1,2,4]triazolo[1,5-a][1,3,5]triazin-5- |
| | yl)amino)ethyl)phenyl)sulfamoyl)-3-chloro-2-hydroxybenzamide; |
| A4 | N-(4-(2-((7-amino-2-(furan-2-yl)-[1,2,4]triazolo[1,5-a][1,3,5]triazin-5- |
| | yl)amino)ethyl)phenyl)-3-chloro-5-fluoro-4-hydroxybenzenesulfonamide; |
| A5 | 5-(N-(4-(2-((7-amino-2-(furan-2-yl)-[1,2,4]triazolo[1,5-a][1,3,5]triazin-5- |
| | yl)amino)ethyl)-3-hydroxyphenyl)sulfamoyl)-3-chloro-2-hydroxybenzamide; |
| A6 | 5-(N-(4-(2-((7-amino-2-(furan-2-yl)-[1,2,4]triazolo[1,5-a][1,3,5]triazin-5- |
| | yl)amino)ethyl)-3-fluorophenyl)sulfamoyl)-3-chloro-2-hydroxybenzamide; |
| A7 | 5-(N-(4-(3-((7-amino-2-(furan-2-yl)-[1,2,4]triazolo[1,5-a][1,3,5]triazin-5- |
| | yl)amino)ethyl)-2-fluorophenyl)sulfamoyl)-3-chloro-2-hydroxybenzamide; |
| A8 | 5-(N-(4-(2-((7-acetamido-2-(furan-2-yl)-[1,2,4]triazolo[1,5-a][1,3,5]triazin-5- |
| | yl)amino)ethyl)phenyl)sulfamoyl)-3-chloro-2-hydroxybenzamide; |
| A9 | Isobutyl(5-((4-((3-carbamoyl-5-chloro-4- |
| | hydroxyphenyl)sulfonamido)phenethyl)amino)-2-(furan-2-yl)- |
| | [1,2,4]triazolo[1,5-a][1,3,5]triazin-7-yl)carbamate; |
| A10 | N-(4-(2-((7-amino-2-(furan-2-yl)-[1,2,4]triazolo[1,5-a][1,3,5]triazin-5- |
| | yl)amino)ethyl)phenyl)-4-hydroxy-3-(trifluoromethyl)benzenesulfonamide; |
| A11 | 5-(N-(4-(3-((7-amino-2-(furan-2-yl)-[1,2,4]triazolo[1,5-a][1,3,5]triazin-5- |
| | yl)amino)propyl)phenyl)sulfamoyl)-3-chloro-2-hydroxybenzamide; |
| A12 | 5-(N-(4-(3-((7-amino-2-(furan-2-yl)-[1,2,4]triazolo[1,5-a][1,3,5]triazin-5- |
| | yl)oxy)propyl)phenyl)sulfamoyl)-3-chloro-2-hydroxybenzamide; |
| A13 | 5-(N-(4-(3-((7-amino-2-(furan-2-yl)-[1,2,4]triazolo[1,5-a][1,3,5]triazin-5- |
| | yl)amino)propyl)-3-hydroxyphenyl)sulfamoyl)-3-chloro-2-hydroxybenzamide; |
| A14 | 5-(N-(4-(3-((7-amino-2-(furan-2-yl)-[1,2,4]triazolo[1,5-a][1,3,5]triazin-5- |
| | yl)amino)propyl)-3-fluorophenyl)sulfamoyl)-3-chloro-2-hydroxybenzamide; |
| A15 | 5-(N-(4-(3-((7-amino-2-(furan-2-yl)-[1,2,4]triazolo[1,5-a][1,3,5]triazin-5- |
| | yl)amino)propyl)-2-fluorophenyl)sulfamoyl)-3-chloro-2-hydroxybenzamide; |
| A16 | 5-(N-(4-(3-((7-amino-2-(furan-2-yl)-[1,2,4]triazolo[1,5-a][1,3,5]triazin-5- |
| | yl)amino)propyl)phenyl)sulfamoyl)-3-chloro-2-methoxybenzamide; |
| A17 | 5-(N-(4-(3-((7-amino-2-(furan-2-yl)-[1,2,4]triazolo[1,5-a][1,3,5]triazin-5- |
| | yl)(methyl)amino)propyl)phenyl)sulfamoyl)-3-chloro-2-hydroxybenzamide; |
| A18 | 5-(N-(4-(4-((7-amino-2-(furan-2-yl)-[1,2,4]triazolo[1,5-a][1,3,5]triazin-5- |
| | yl)amino)butyl)phenyl)sulfamoyl)-3-chloro-2-hydroxybenzamide; |
| A19 | 5-(N-(4-(3-((5-amino-2-(furan-2-yl)-[1,2,4]triazolo[1,5-c]pyrimidin-7- |
| | yl)amino)propyl)phenyl)sulfamoyl)-3-chloro-2-hydroxybenzamide; |
| A20 | 5-(N-(4-(3-((7-amino-2-(furan-2-yl)-[1,2,4]triazolo[1,5-a]pyrimidin-5- |
| | yl)amino)propyl)phenyl)sulfamoyl)-3-chloro-2-hydroxybenzamide; |
| A21 | 5-(N-(4-(3-((7-amino-2-(furan-2-yl)-[1,2,4]triazolo[1,5-a]pyrimidin-5- |
| | yl)oxy)propyl)phenyl)sulfamoyl)-3-chloro-2-hydroxybenzamide; |
| A22 | 5-(N-(4-(3-((7-amino-2-(furan-2-yl)-[1,2,4]triazolo[1,5-a]pyrimidin-5- |
| | yl)amino)propyl)-3-fluorophenyl)sulfamoyl)-3-chloro-2-hydroxybenzamide; |
| A23 | 5-(N-(4-(3-((7-amino-2-(furan-2-yl)-[1,2,4]triazolo[1,5-a]pyrimidin-5- |
| | yl)amino)propyl)-2-fluorophenyl)sulfamoyl)-3-chloro-2-hydroxybenzamide; |
| A24 | 5-(N-(4-(3-((7-amino-2-(furan-2-yl)pyrazolo[1,5-a]pyrimidin-5- |
| | yl)amino)propyl)phenyl)sulfamoyl)-3-chloro-2-hydroxybenzamide; |
| A25 | Methyl ((4-(N-(4-(3-((7-amino-2-(furan-2-yl)-[1,2,4]triazolo[1,5-a][1,3,5]triazin-5- |
| | yl)amino)propyl)phenyl)sulfamoyl)-2-carbamoyl-6- |
| | chlorophenoxy)carbonyl)valinate; |
| A26 | 5-(N-(4-(3-((7-amino-2-(furan-2-yl)-[1,2,4]triazolo[1,5-a][1,3,5]triazin-5- |
| | yl)amino)propyl)phenyl)sulfamoyl)-3-chloro-2-hydroxybenzamide sodium salt; |
| A27 | Ethyl (5-((3-(4-((3-chloro-N-(ethoxycarbonyl)-5-((ethoxycarbonyl)carbamoyl)-4- |
| | ((ethoxycarbonyl)oxy)phenyl)sulfonamido)phenyl)propyl)(ethoxycarbonyl)amino)- |
| | 2-(furan-2-yl)-[1,2,4]triazolo[1,5-a][1,3,5]triazin-7-yl)(ethoxycarbonyl)carbamate; |
| A28 | Tert-Butyl (1-(5-(N-(4-(3-((7-amino-2-(furan-2-yl)-[1,2,4]triazolo[1,5- |
| | a][1,3,5]triazin-5-yl)amino)propyl)phenyl)sulfamoyl)-3-chloro-2- |
| | hydroxybenzamido)-3-methyl-1-oxobutan-2-yl)carbamate; |
| A29 | Hexyl (7-amino-2-(furan-2-yl)-[1,2,4]triazolo[1,5-a][1,3,5]triazin-5-yl)(3-(4-((3- |
| | carbamoyl-5-chloro-4-hydroxyphenyl)sulfonamido)phenyl)propyl)carbamate; |
| A30 | 3-(Hexadecyloxy)propyl (7-amino-2-(furan-2-yl)-[1,2,4]triazolo[1,5- |
| | a][1,3,5]triazin-5-yl)(3-(4-((3-carbamoyl-5-chloro-4- |
| | hydroxyphenyl)sulfonamido)phenyl)propyl)carbamate; |
| A31 | 1-(((7-Amino-2-(furan-2-yl)-[1,2,4]triazolo[1,5-a][1,3,5]triazin-5-yl)(3-(4-((3- |
| | carbamoyl-5-chloro-4- |
| | hydroxyphenyl)sulfonamido)phenyl)propyl)carbamoyl)oxy)ethyl 3- |
| | methylbutanoate; |
| B1 | N-(4-(2-((7-amino-2-(furan-2-yl)-[1,2,4]triazolo[1,5-a][1,3,5]triazin-5- |
| | yl)amino)ethyl)phenyl)-7-chloro-2-oxoindoline-5-sulfonamide; |
| B2 | N-(4-(2-((7-amino-2-(furan-2-yl)-[1,2,4]triazolo[1,5-a][1,3,5]triazin-5- |
| | yl)amino)ethyl)phenyl)-3,3,7-trichloro-2-oxoindoline-5-sulfonamide; |
| B3 | N-(4-(2-((7-amino-2-(furan-2-yl)-[1,2,4]triazolo[1,5-a][1,3,5]triazin-5- |
| | yl)amino)ethyl)phenyl)-8-chloro-4-oxo-1,4-dihydroquinoline-6-sulfonamide; |
| B4 | N-(4-(3-((7-amino-2-(furan-2-yl)-[1,2,4]triazolo[1,5-a][1,3,5]triazin-5- |
| | yl)amino)propyl)phenyl)-8-chloro-2,4-dioxo-3,4-dihydro-2H- |
| | benzo[e][1,3]oxazine-6-sulfonamide; |
| C1 | 5-(N-(4-((2-((7-amino-2-(furan-2-yl)-[1,2,4]triazolo[1,5-a][1,3,5]triazin-5- |
| | yl)amino)ethyl)amino)phenyl)sulfamoyl)-3-chloro-2-hydroxybenzamide; |
| C2 | 5-(N-(3-((2-((7-amino-2-(furan-2-yl)-[1,2,4]triazolo[1,5-a][1,3,5]triazin-5- |
| | yl)amino)ethyl)amino)phenyl)sulfamoyl)-3-chloro-2-hydroxybenzamide; |
| D1 | 5-(N-(1-(2-((7-amino-2-(furan-2-yl)-[1,2,4]triazolo[1,5-a][1,3,5]triazin-5- |
| | yl)amino)ethyl)-1H-indol-6-yl)sulfamoyl)-3-chloro-2-hydroxybenzamide; |
| D2 | 5-(N-(1-(2-((7-amino-2-(furan-2-yl)-[1,2,4]triazolo[1,5-a][1,3,5]triazin-5- |
| | yl)amino)ethyl)-1H-indazol-4-yl)sulfamoyl)-3-chloro-2-hydroxybenzamide; |
| E1 | 5-(N-(6-(2-((7-amino-2-(furan-2-yl)-[1,2,4]triazolo[1,5-a][1,3,5]triazin-5- |
| | yl)amino)ethyl)pyridin-3-yl)sulfamoyl)-3-chloro-2-hydroxybenzamide; and |
| E2 | 5-(N-(6-(2-((7-amino-2-(furan-2-yl)-[1,2,4]triazolo[1,5-a]pyrimidin-5- |
| | yl)amino)ethyl)pyridin-3-yl)sulfamoyl)-3-chloro-2-hydroxybenzamide; |

or a pharmaceutically acceptable salt or pro rug thereof.

35. A pharmaceutical composition comprising a compound of according to claim 1 or a pharmaceutically acceptable carrier.

36. (canceled)

37. A method for treating or delaying progression of diseases or disorders mediated by CD73 and/or Adenosine receptors (A2aR and/or A2bR) in a subject, the method comprising administering to the subject a therapeutically effective amount of a compound according to claim 1.

38. The method of claim 37, wherein the diseases or disorders mediated by CD73 and/or Adenosine receptors (A2aR and/or A2bR) is a cancer.

39. The method of claim 38, wherein the cancer is brain gliomas, glioblastomas, astrocytomas, multiforme, bannayan-Zonana syndrome, Cowden disease, Lhermitte-Duclos disease, breast cancer, colon cancer, head and neck cancer, kidney, liver, lung cancer, bone cancer, colorectal cancer, germ cell cancer, melanoma, ovarian cancer, pancreatic cancer, adenocarcinoma, ductal adenocarcinoma, adenosquamous carcinoma, acinar cell carcinoma, glucagonoma, insulinoma, prostate, sarcoma and thyroid cancer, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, immunoblastic large cell leukemia, mantle cell leukemia, multiple myeloma, megakaryoblastic leukemia, multiple myeloma, acute megakaryocytic leukemia, promyelocytic leukemia, erythroleukemia, malignant lymphoma, Hodgkin's lymphoma, non-Hodgkin's lymphoma, lymphoblastic T cell lymphoma, Burkitt's lymphoma, follicular lymphoma, neuroblastoma, bladder cancer, urothelial cancer, vulval cancer, uterine/cervical cancer, endometrial cancer, renal cancer, mesothelioma, esophageal cancer, salivary gland cancer, hepatocellular cancer, gastric cancer, nasopharyngeal cancer, buccal cancer, cancer of the mouth, GIST (gastrointestinal stromal tumor), neuroendocrine cancers, testicular cancer or virus-related cancer.

40.-45. (canceled)
